(19) United States
(12) Patent Application Publication
(10) Pub. No.: US 2019/0189925 A1
(43) Pub. Date: Jun. 20, 2019

(54) ORGANIC SEMICONDUCTORS

(71) Applicant: MERCK PATENT GMBH, Darmstadt (DE)

(72) Inventors: Changsheng WANG, Eastleigh (GB); William MITCHELL, Chandler's Ford (GB)

(73) Assignee: MERCK PATENT GMBH, Darmstadt (DE)

(21) Appl. No.: 16/329,267

(22) PCT Filed: Aug. 28, 2017
(86) PCT No.: PCT/EP2017/071520

§ 371 Date: Feb. 28, 2019

(30) Foreign Application Priority Data
Aug. 29, 2016 (EP) 16186094.5

(51) Int. Cl. H01L 51/00; C07D 513/04; C08G 61/12

(52) U.S. Cl. CPC H01L 51/0036; H01L 51/0046; C07D 513/04; H01L 51/4253; H01L 51/0068; C08G 61/126; H01L 51/0043; H01L 51/0071

(57) ABSTRACT

The invention relates to novel organic semiconducting (OSC) compounds containing one or more 1,3-dithiolo[5,6-f]benzo-2,1,3-thiadiazole (“DTBTz”) or 1,3-dithiolo[6,7-g]quinoxaline (“DTQ”) units or derivatives thereof, to methods for their preparation and educts or intermediates used therein, to compositions and formulations containing them, to the use of the compounds and compositions as organic semiconductors in, or for the preparation of, organic electronic (OE) devices, especially organic photovoltaic (OPV) devices, perovskite-based solar cell (PSC) devices, organic photodetectors (OPD), organic field effect transistors (OFET) and organic light emitting diodes (OLED), and to OE devices comprising these compounds or compositions.

### TECHNICAL FIELD

The invention relates to novel organic semiconducting (OSC) compounds containing one or more 1,3-dithiolo[5,6-f]benzo-2,1,3-thiadiazole (“DTBTz”) or 1,3-dithiolo[6,7-g]quinoxaline (“DTQ”) units or derivatives thereof, to methods for their preparation and educts or intermediates used therein, to compositions and formulations containing them, to the use of the compounds and compositions as organic semiconductors in, or for the preparation of, organic electronic (OE) devices, especially organic photovoltaic (OPV) devices, perovskite-based solar cell (PSC) devices, organic photodetectors (OPD), organic field effect transistors (OFET) and organic light emitting diodes (OLED), and to OE devices comprising these compounds or compositions.

### BACKGROUND

In recent years, there has been development of OSC materials in order to produce more versatile, lower cost electronic devices. Such materials find application in a wide range of devices or apparatus, including organic field effect transistors (OFETs), organic light emitting diodes (OLEDs), organic photodetectors (OPDs), organic photovoltaic (OPV) cells, perovskite-based solar cell (PSC) devices, sensors, memory elements and logic circuits to name just a few. The OSC materials are typically present in the electronic device in the form of a thin layer, for example of between 50 and 300 nm thickness.

OSC materials are receiving ever-growing attention mostly due to their lucrative commercial prospects in organic electronics manufactured by cost effective solution processing technology at low temperature. It is generally believed that OSCs have a number of advantage over their inorganic counterparts, such as the potential of fabricating lightweight flexible backplanes, the opportunity to make large area displays using low-cost, high speed solution based fabrication techniques, and their optical and electronic properties being fine-tuneable via rational chemical structure modifications.

One particular area of importance is organic photovoltaics (OPV). Conjugated OSC polymers and OSC small molecules have found use in OPVs, mainly in the photoactive layer, as they allow devices to be manufactured by solution-processing techniques such as spin casting, dip coating or ink jet printing. Solution processing can be carried out cheaper and on a larger scale compared to the evaporative techniques used to make inorganic thin film devices. Currently, polymer based photovoltaic devices are achieving efficiencies above 10%.

In photoactive layers containing a blend of an n-type OSC and a p-type OSC, typically a π-conjugated polymer, forming a bulk-heterojunction (BHJ), the π-conjugated polymer serves as the main absorber of the solar energy. Therefore a low band gap is a basic requirement for the polymer to absorb the maximum of the solar spectrum.

Thus, for use as donor OSC in OPV cells and OPDs, the conjugated polymer should have a low bandgap, which enables improved light harvesting by the photoactive layer and can lead to higher power conversion efficiency.

Polymerising π-π-donor-acceptor (D-A) monomers to synthesize D-A copolymers through transition metal catalysed polycondensation is a known strategy to achieve low bandgap semiconducting polymers for OPV and OPD applications. Conjugated D-A copolymers have also been found to demonstrate high charge carrier mobilities in OTFTs. It is generally accepted that the alternating D-A structure facilitates stronger intermolecular interactions, leading to smaller π-π-stacking distance and efficient intermolecular charge transfer due to static attractions between the donor and the acceptor monomer units.

To date, a large number of conjugated i-structures have been synthesized which can be used as monomers for preparing conjugated OSC polymers. However, electron donor units remain overwhelmingly dominant in the pool of monomers mainly due to the relative ease of synthetic accessibility of building blocks and precursors. In contrast, there are only few electron acceptor units or monomers available.

Therefore, it is desired to increase the pool of electron acceptors by adding electron deficient n-units to make further promising D-A copolymers available.

Another particular area of importance are OFETs. The performance of OFET devices is principally based upon the charge carrier mobility of the semiconducting material and the current on/off ratio, so the ideal semiconductor should have a low conductivity in the off state, combined with high charge carrier mobility (>1×10⁻¹ cm² V⁻¹ s⁻¹). In addition, it is important that the semiconducting material is stable to oxidation i.e. it has a high ionisation potential, as oxidation leads to reduced device performance, like for example increased off current and threshold voltage shift. Further requirements for the semiconducting material are good processibility, especially for large-scale production of thin layers and desired patterns, and high stability, film uniformity and integrity of the organic semiconductor layer.

However, the OSC materials disclosed in prior art for use in OE devices do still have several drawbacks, such as poor solubility in solvents suitable for mass production, relatively low device performance such as inadequate charge-carrier mobility for commercial application for example in transistors, modest thermal, photo and electrical stability, poor long term stability and non-reproducible film forming properties.

Therefore there is still a need for OSC materials for use in OE devices like OPVs, OPDs and OFETs, which have advantageous properties, in particular good processibility, high solubility in organic solvents, good structural organization and film-forming properties. In addition, the OSC materials should be easy to synthesize, especially by methods suitable for mass production. For use in OPV cells, the OSC materials should especially have a low bandgap, which enables improved light harvesting by the photoactive layer and can lead to higher cell efficiencies, high stability and long lifetime. For use in OFETs the OSC materials should especially have high charge-carrier mobility, high on/off ratio in transistor devices, high oxidative stability and long lifetime.

It was an aim of the present invention to provide new OSC compounds, including p-type and n-type OSCs, which can overcome the drawbacks of the OSCs from prior art, and which provide one or more of the above-mentioned advantageous properties, especially easy synthesis by methods suitable for mass production, good processibility, high stability, long lifetime in OE devices, good solubility in organic solvents, high charge carrier mobility, and a low bandgap. Another aim of the invention was to extend the pool of OSC materials and p-type and n-type OSCs available to the expert. Other aims of the present invention are immediately evident to the expert from the following detailed description.

The inventors of the present invention have found that one or more of the above aims can be achieved by providing compounds as disclosed and claimed hereinafter. These contain a 1,3-dithiolo[5,6-f]benzo-2,1,3-thiadiazole (“DTBTz”) or 1,3-dithiolo[6,7-g]quinoxaline (“DTQ”) unit or a derivative thereof, as shown in formula I.

It has been found that compounds comprising such a DTBTz or DTQ unit can be used as OSCs which show advantageous properties as described above.

### SUMMARY

The invention relates to a compound comprising one or more divalent units of formula I

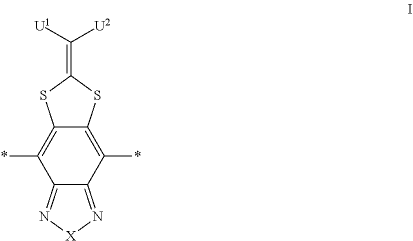

wherein the individual radicals, independently of each other and on each occurrence identically or differently, have the following meanings
X O, S, Se, Te, —NR—, —PR—, —P(═O)—, —P(OR)—, —P(O)(OR)— or —CR¹═CR²—,
U¹, U² an electron withdrawing group, preferably selected from CN, C(═O)R or C(═O)OR, or U¹ and U² together form a carbocyclic, heterocyclic, aromatic or heteroaromatic ring having 4 to 15 ring atoms that is optionally substituted by one or more groups L, R¹ or R²,
R H or straight-chain, branched or cyclic alkyl with 1 to 30, preferably 1 to 25, C atoms, in which one or more CH₂ groups are optionally replaced by —O—, —S—, —C(═O)—, —C(═S)—, —C(═O)—O—, —O—C(═O)—, —NR⁰—, —SiR⁰R⁰⁰—, —CF₂—, —CR⁰═CR⁰⁰—, —CY¹═CY²— or —C≡C— in such a manner that O and/or S atoms are not linked directly to one another, and in which one or more H atoms are optionally replaced by F, Cl, Br, I or CN, and in which one or more CH₂ or CH₃ groups are optionally replaced by a cationic or anionic group, or aryl, heteroaryl, arylalkyl, heteroarylalkyl, aryloxy or heteroaryloxy, wherein each of the aforementioned cyclic groups has 5 to 20 ring atoms, is mono- or polycyclic, does optionally contain fused rings, and is unsubstituted or substituted by one or more identical or different groups L,
R^{1,2} H, F, Cl, CN or straight-chain, branched or cyclic alkyl with 1 to 30, preferably 1 to 20, C atoms, in which one or more CH₂ groups are optionally replaced by —O—, —S—, —C(═O)—, —C(═S)—, —C(═O)—O—, —O—C(═O)—, —NR⁰—, —SiR⁰R⁰⁰—, —CF₂—, —CR⁰═CR⁰⁰—, —CY¹═CY²— or —C≡C— in such a manner that O and/or S atoms are not linked directly to one another, and in which one or more H atoms are optionally replaced by F, Cl, Br, I or CN, and in which one or more CH₂ or CH₃ groups are optionally replaced by a cationic or anionic group, or aryl, heteroaryl, arylalkyl, heteroarylalkyl, aryloxy or heteroaryloxy, wherein each of the aforementioned cyclic groups has 5 to 20 ring atoms, is mono- or polycyclic, does optionally contain fused rings, and is unsubstituted or substituted by one or more identical or different groups L,
   or R¹ and R² form an aromatic or heteroaromatic ring system that is fused to the pyrazine ring to which R¹ and R² are attached, has 5 to 20 ring atoms, is mono- or polycyclic, optionally contains fused rings, and is unsubstituted or substituted by one or more identical or different groups L,
L F, Cl, —CN, —NC, —NCO, —NCS, —OCN, —SCN, R⁰, OR⁰, SR⁰, —C(═O)X⁰, —C(═O)R⁰, —C(═O)—OR⁰, —O—C(═O)—R⁰, —NH₂, —NHR⁰, —NR⁰R⁰⁰, —C(═O)NHR⁰, —C(═O)NR⁰R⁰⁰, —SO₃R⁰, —SO₂R⁰, —OH, —NO₂, —CF₃, —SF₅, or optionally substituted silyl, or carbyl or hydrocarbyl with 1 to 30, preferably 1 to 20 C atoms that is optionally substituted and optionally comprises one or more hetero atoms, preferably F, —CN, R⁰, —OR⁰, —SR⁰, —C(═O)—R⁰, —C(═O)—OR⁰, —O—C(═O)—R⁰, —O—C(═O)—OR⁰, —C(═O)—NHR⁰, —C(═O)—NR⁰R⁰⁰,
Y¹, Y² H, F, Cl or CN,
X⁰ halogen, preferably F or Cl,
R⁰, R⁰⁰ H or straight-chain or branched alkyl with 1 to 20, preferably 1 to 12, C atoms that is optionally fluorinated.

The invention further relates to the use of the units of formula I in or as repeating units in conjugated polymers.

A compound comprising one or more units of formula I is hereinafter also referred to as “compound according to the (present) invention”.

The invention further relates to a compound according to the present invention which is a conjugated polymer comprising one or more repeating units of formula I.

The invention further relates to a conjugated polymer comprising one or more repeating units of formula I, and additionally comprises one or more distinct arylene or heteroarylene units that have from 5 to 20 ring atoms, are mono- or polycyclic, do optionally contain fused rings, are unsubstituted or substituted by one or more identical or different groups L, R¹ or R², and are either selected of formula I or are structurally different from formula I, and wherein all the aforementioned units are directly connected to each other.

The invention further relates to a conjugated polymer as described above wherein one or more of the additional arylene or heteroarylene units have electron donor property. The invention further relates to a conjugated polymer as described above wherein one or more of the additional arylene or heteroarylene units have electron acceptor property.

The invention further relates to compound according to the present invention which is a small molecule or oligomer comprising one or more divalent units of formula I.

The invention further relates to a compound according to the present invention which is a monomer comprising a divalent unit of formula I, optionally further comprising one or more additional arylene or heteroarylene units, and further comprising one or more reactive groups which can be reacted to form a conjugated polymer as described above and below.

The invention further relates to a compound according to the present invention which is a small molecule or oligomer comprising one or more divalent units of formula I and further comprising one or more electron-withdrawing groups which can be laterally or terminally attached to the unit of formula I.

The invention further relates to the use of a compound according to the present invention as electron donor or p-type semiconductor, or as electron acceptor or n-type semiconductor.

The invention further relates to the use of a compound according to the present invention as electron donor or electron acceptor component in a semiconducting material, formulation, polymer blend, device or component of a device.

The invention further relates to a semiconducting material, formulation, polymer blend, device or component of a device comprising a compound according to the present invention as electron donor component, and preferably further comprising one or more compounds having electron acceptor properties.

The invention further relates to a semiconducting material, formulation, polymer blend, device or component of a device comprising a compound according to the present invention as electron acceptor component, and preferably further comprising one or more compounds having electron donor properties.

The invention further relates to a composition, which may also be a polymer blend, comprising one or more compounds according to the present invention, and further comprising one or more additional compounds selected from compounds having one or more of semiconducting, charge transport, hole or electron transport, hole or electron blocking, electrically conducting, photoconducting or light emitting properties.

The invention further relates to a composition comprising one or more compounds according to the present invention, and further comprising one or more n-type organic semiconductors, preferably selected from fullerenes or substituted fullerenes.

The invention further relates to a composition comprising a compound according to the present invention, and further comprising one or more electron donors or p-type semiconductors, preferably selected from conjugated polymers.

The invention further relates to a composition comprising a first n-type semiconductor which is a compound according to the present invention, a second n-type semiconductor, which is preferably a fullerene or fullerene derivative, and a p-type semiconductor, which is a conjugated polymer.

The invention further relates to a bulk heterojunction (BHJ) formed from a composition comprising a compound according to the present invention as electron acceptor or n-type semiconductor, and one or more compounds which are electron donor or p-type semiconductors and are preferably selected from conjugated polymers.

The invention further relates to a formulation comprising one or more compounds or a composition according to the present invention, and further comprising one or more solvents, preferably selected from organic solvents.

The invention further relates to an organic semiconducting formulation comprising one or more compounds according to the present invention, and further comprising one or more organic binders or precursors thereof, preferably having a permittivity ε at 1,000 Hz and 20° C. of 3.3 or less, and optionally one or more solvents preferably selected from organic solvents.

The invention further relates to an optical, electrooptical, electronic, electroluminescent or photoluminescent device, or a component thereof, or an assembly comprising it, which is prepared using a formulation according to the present invention.

The invention further relates to the use of a compound or composition according to the present invention as semiconducting, charge transport, electrically conducting, photoconducting or light emitting material, or in an optical, electrooptical, electronic, electroluminescent or photoluminescent device, or in a component of such a device or in an assembly comprising such a device or component

The invention further relates to a semiconducting, charge transport, electrically conducting, photoconducting or light emitting material comprising a compound or composition according to the present invention.

The invention further relates to an optical, electrooptical, electronic, electroluminescent or photoluminescent device, or a component thereof, or an assembly comprising it, which comprises a compound or composition according to the present invention, or comprises a semiconducting, charge transport, electrically conducting, photoconducting or light emitting material according to the present invention.

The optical, electrooptical, electronic, electroluminescent and photoluminescent device includes, without limitation, organic field effect transistors (OFET), organic thin film transistors (OTFT), organic light emitting diodes (OLED), organic light emitting transistors (OLET), organic photovoltaic devices (OPV), organic photodetectors (OPD), organic solar cells, dye-sensitized solar cells (DSSC), perovskite-based solar cells (PSC), laser diodes, Schottky diodes, photoconductors and photodetectors.

Preferred devices are OFETs, OTFTs, OPVs, PSCs, OPDs and OLEDs, in particular OTFTs, PSCs, OPDs and bulk heterojunction (BHJ) OPVs or inverted BHJ OPVs.

Further preferred is the use of a compound or composition according to the present invention as dye in a DSSC or a PSC. Further preferred is a DSSC or PSC comprising a compound or composition according to the present invention.

The component of the above devices includes, without limitation, charge injection layers, charge transport layers, interlayers, planarising layers, antistatic films, polymer electrolyte membranes (PEM), conducting substrates and conducting patterns.

The assembly comprising such a device or component includes, without limitation, integrated circuits (IC), radio frequency identification (RFID) tags or security markings or security devices containing them, flat panel displays or backlights thereof, electrophotographic devices, electrophotographic recording devices, organic memory devices, sensor devices, biosensors and biochips.

In addition the compounds, compositions and formulations of the present invention can be used as electrode materials in batteries and in components or devices for detecting and discriminating DNA sequences.

The invention further relates to a bulk heterojunction which comprises, or is being formed from, a composition comprising one or more compounds according to the present invention and one or more n-type organic semiconductors that are preferably selected from fullerenes or substituted fullerenes. The invention further relates to a bulk heterojunction (BHJ) OPV device or inverted BHJ OPV device, comprising such a bulk heterojunction.

### Terms and Definitions

As used herein, the term “polymer” will be understood to mean a molecule of high relative molecular mass, the structure of which essentially comprises multiple repetitions of units derived, actually or conceptually, from molecules of low relative molecular mass (Pure Appl. Chem., 1996, 68, 2291). The term “oligomer” will be understood to mean a molecule of intermediate relative molecular mass, the structure of which essentially comprises a small plurality of units derived, actually or conceptually, from molecules of lower relative molecular mass (Pure Appl. Chem., 1996, 68, 2291). In a preferred meaning as used herein present invention a polymer will be understood to mean a compound having >1, i.e. at least 2 repeat units, preferably ≥5 repeat units, and an oligomer will be understood to mean a compound with >1 and <10, preferably <5, repeat units.

Further, as used herein, the term “polymer” will be understood to mean a molecule that encompasses a backbone (also referred to as “main chain”) of one or more distinct types of repeat units (the smallest constitutional unit of the molecule) and is inclusive of the commonly known terms “oligomer”, “copolymer”, “homopolymer”, “random polymer” and the like. Further, it will be understood that the term polymer is inclusive of, in addition to the polymer itself, residues from initiators, catalysts and other elements attendant to the synthesis of such a polymer, where such residues are understood as not being covalently incorporated thereto. Further, such residues and other elements, while normally removed during post polymerization purification processes, are typically mixed or co-mingled with the polymer such that they generally remain with the polymer when it is transferred between vessels or between solvents or dispersion media.

As used herein, in a formula showing a polymer or a repeat unit, like for example a unit of formula I or a polymer of formula III or IV or their subformulae, an asterisk (*) will be understood to mean a chemical linkage to an adjacent unit or to a terminal group in the polymer backbone. In a ring, like for example a benzene or thiophene ring, an asterisk (*) will be understood to mean a C atom that is fused to an adjacent ring.

As used herein, the terms “repeat unit”, “repeating unit” and “monomeric unit” are used interchangeably and will be understood to mean the constitutional repeating unit (CRU), which is the smallest constitutional unit the repetition of which constitutes a regular macromolecule, a regular oligomer molecule, a regular block or a regular chain (Pure Appl. Chem., 1996, 68, 2291). As further used herein, the term “unit” will be understood to mean a structural unit which can be a repeating unit on its own, or can together with other units form a constitutional repeating unit.

As used herein, a “terminal group” will be understood to mean a group that terminates a polymer backbone. The expression “in terminal position in the backbone” will be understood to mean a divalent unit or repeat unit that is linked at one side to such a terminal group and at the other side to another repeat unit. Such terminal groups include endcap groups, or reactive groups that are attached to a monomer forming the polymer backbone which did not participate in the polymerisation reaction, like for example a group having the meaning of R⁵ or R⁶ as defined below.

As used herein, the term “endcap group” will be understood to mean a group that is attached to, or replacing, a terminal group of the polymer backbone. The endcap group can be introduced into the polymer by an endcapping process. Endcapping can be carried out for example by reacting the terminal groups of the polymer backbone with a monofunctional compound (“endcapper”) like for example an alkyl- or arylhalide, an alkyl- or arylstannane or an alkyl- or arylboronate. The endcapper can be added for example after the polymerisation reaction. Alternatively the endcapper can be added in situ to the reaction mixture before or during the polymerisation reaction. In situ addition of an endcapper can also be used to terminate the polymerisation reaction and thus control the molecular weight of the forming polymer. Typical endcap groups are for example H, phenyl and lower alkyl.

As used herein, the term “small molecule” will be understood to mean a monomeric compound which typically does not contain a reactive group by which it can be reacted to form a polymer, and which is designated to be used in monomeric form. In contrast thereto, the term “monomer” unless stated otherwise will be understood to mean a monomeric compound that carries one or more reactive functional groups by which it can be reacted to form a polymer.

As used herein, the terms “donor” or “donating” and “acceptor” or “accepting” will be understood to mean an electron donor or electron acceptor, respectively. “Electron donor” will be understood to mean a chemical entity that donates electrons to another compound or another group of atoms of a compound. “Electron acceptor” will be understood to mean a chemical entity that accepts electrons transferred to it from another compound or another group of atoms of a compound. See also International Union of Pure and Applied Chemistry, Compendium of Chemical Technology, Gold Book, Version 2.3.2, 19. August 2012, pages 477 and 480.

As used herein, the term “n-type” or “n-type semiconductor” will be understood to mean an extrinsic semiconductor in which the conduction electron density is in excess of the mobile hole density, and the term “p-type” or “p-type semiconductor” will be understood to mean an extrinsic semiconductor in which mobile hole density is in excess of the conduction electron density (see also, J. Thewlis, Concise Dictionary of Physics, Pergamon Press, Oxford, 1973).

As used herein, the term “leaving group” will be understood to mean an atom or group (which may be charged or uncharged) that becomes detached from an atom in what is considered to be the residual or main part of the molecule taking part in a specified reaction (see also Pure Appl. Chem., 1994, 66, 1134).

As used herein, the term “conjugated” will be understood to mean a compound (for example a polymer) that contains mainly C atoms with sp²-hybridisation (or optionally also sp-hybridisation), and wherein these C atoms may also be replaced by hetero atoms. In the simplest case this is for example a compound with alternating C—C single and double (or triple) bonds, but is also inclusive of compounds with aromatic units like for example 1,4-phenylene. The term “mainly” in this connection will be understood to mean that a compound with naturally (spontaneously) occurring defects, or with defects included by design, which may lead to interruption of the conjugation, is still regarded as a conjugated compound.

As used herein, unless stated otherwise the molecular weight is given as the number average molecular weight Mₙ or weight average molecular weight M_{W}, which is determined by gel permeation chromatography (GPC) against polystyrene standards in eluent solvents such as tetrahydrofuran, trichloromethane (TCM, chloroform), chlorobenzene or 1,2,4-trichlorobenzene. Unless stated otherwise, 1,2,4-trichlorobenzene is used as solvent. The degree of polymerization, also referred to as total number of repeat units, n, will be understood to mean the number average degree of polymerization given as n=Mₙ/M_{U}, wherein Mₙ is the number average molecular weight and M_{U} is the molecular weight of the single repeat unit, see J. M. G. Cowie, Polymers: Chemistry & Physics of Modern Materials, Blackie, Glasgow, 1991.

As used herein, the term “carbyl group” will be understood to mean any monovalent or multivalent organic moiety which comprises at least one carbon atom either without any non-carbon atoms (like for example —C≡C—), or optionally combined with at least one non-carbon atom such as B, N, O, S, P, Si, Se, As, Te or Ge (for example carbonyl etc.).

As used herein, the term “hydrocarbyl group” will be understood to mean a carbyl group that does additionally contain one or more H atoms and optionally contains one or more hetero atoms like for example B, N, O, S, P, Si, Se, As, Te or Ge.

As used herein, the term “hetero atom” will be understood to mean an atom in an organic compound that is not a H- or C-atom, and preferably will be understood to mean B, N, O, S, P, Si, Se, As, Te or Ge.

A carbyl or hydrocarbyl group comprising a chain of 3 or more C atoms may be straight-chain, branched and/or cyclic, and may include spiro-connected and/or fused rings.

Preferred carbyl and hydrocarbyl groups include alkyl, alkoxy, thioalkyl, alkylcarbonyl, alkoxycarbonyl, alkylcarbonyloxy and alkoxycarbonyloxy, each of which is optionally substituted and has 1 to 40, preferably 1 to 25, very preferably 1 to 18 C atoms, furthermore optionally substituted aryl or aryloxy having 6 to 40, preferably 6 to 25 C atoms, furthermore alkylaryloxy, arylcarbonyl, aryloxycarbonyl, arylcarbonyloxy and aryloxycarbonyloxy, each of which is optionally substituted and has 6 to 40, preferably 7 to 40 C atoms, wherein all these groups do optionally contain one or more hetero atoms, preferably selected from B, N, O, S, P, Si, Se, As, Te and Ge.

Further preferred carbyl and hydrocarbyl group include for example: a C₁-C₄₀ alkyl group, a C₁-C₄₀ fluoroalkyl group, a C₁-C₄₀ alkoxy or oxaalkyl group, a C₂-C₄₀ alkenyl group, a C₂-C₄₀ alkynyl group, a C₃-C₄₀ allyl group, a C₄-C₄₀ alkyldienyl group, a C₄-C₄₀ polyenyl group, a C₂-C₄₀ ketone group, a C₂-C₄₀ ester group, a C₆-C₁₈ aryl group, a C₆-C₄₀ alkylaryl group, a C₆-C₄₀ arylalkyl group, a C₄-C₄₀ cycloalkyl group, a C₄-C₄₀ cycloalkenyl group, and the like. Preferred among the foregoing groups are a C₁-C₂₀ alkyl group, a C₁-C₂₀ fluoroalkyl group, a C₂-C₂₀ alkenyl group, a C₂-C₂₀ alkynyl group, a C₃-C₂₀ allyl group, a C₄-C₂₀ alkyldienyl group, a C₂-C₂₀ ketone group, a C₂-C₂₀ ester group, a C₆-C₁₂ aryl group, and a C₄-C₂₀ polyenyl group, respectively.

Also included are combinations of groups having carbon atoms and groups having hetero atoms, like e.g. an alkynyl group, preferably ethynyl, that is substituted with a silyl group, preferably a trialkylsilyl group.

The carbyl or hydrocarbyl group may be an acyclic group or a cyclic group. Where the carbyl or hydrocarbyl group is an acyclic group, it may be straight-chain or branched. Where the carbyl or hydrocarbyl group is a cyclic group, it may be a non-aromatic carbocyclic or heterocyclic group, or an aryl or heteroaryl group.

A non-aromatic carbocyclic group as referred to above and below is saturated or unsaturated and preferably has 4 to 30 ring C atoms. A non-aromatic heterocyclic group as referred to above and below preferably has 4 to 30 ring C atoms, wherein one or more of the C ring atoms are optionally replaced by a hetero atom, preferably selected from N, O, S, Si and Se, or by a —S(O)— or —S(O)₂— group. The non-aromatic carbo- and heterocyclic groups are mono- or polycyclic, may also contain fused rings, preferably contain 1, 2, 3 or 4 fused or unfused rings, and are optionally substituted with one or more groups L, wherein L is selected from F, Cl, —CN, —NC, —NCO, —NCS, —OCN, —SCN, —R⁰, —OR⁰, —SR⁰, —C(═O)X⁰, —C(═O)R⁰, —C(═O)—OR⁰, —O—C(═O)R⁰, —NH₂, —NHR⁰, —NR⁰R⁰⁰, —C(═O)NHR⁰, —C(═O)NR⁰R⁰⁰, —SO₃H, —SO₂R⁰, —OH, —NO₂, —CF₃, —SF₅, or optionally substituted silyl, or carbyl or hydrocarbyl with 1 to 30, preferably 1 to 20, C atoms that is optionally substituted and optionally comprises one or more hetero atoms, wherein X⁰ is halogen, preferably F or Cl, and R⁰, R⁰⁰ denote H or straight-chain or branched alkyl with 1 to 20, preferably 1 to 12 C atoms that is optionally fluorinated.

Preferably L is selected from F, —CN, R, —OR, —SR, —C(═O)—R, —C(═O)—OR, —O—C(═O)—R, —O—C(═O)—OR, —C(═O)—NHR, —C(═O)—NRRⁿ, wherein R and Rⁿ are independently of each other straight-chain or branched alkyl with 1 to 25 C atoms that is optionally fluorinated.

Further preferred substituents L are selected from F or alkyl, alkoxy, oxaalkyl, thioalkyl, fluoroalkyl and fluoroalkoxy with 1 to 16 C atoms, or alkylcarbonyl, alkylcarbonyloxy, alkxoycarbonyl, alkenyl or alkynyl with 2 to 16 C atoms (including the carbonyl-C-atom).

Preferred non-aromatic carbocyclic or heterocyclic groups are tetrahydrofuran, indane, pyran, pyrrolidine, piperidine, cyclopentane, cyclohexane, cycloheptane, cyclopentanone, cyclohexanone, dihydro-furan-2-one, tetrahydro-pyran-2-one and oxepan-2-one.

An aryl group as referred to above and below preferably has 4 to 30 ring C atoms, is mono- or polycyclic and may also contain fused rings, preferably contains 1, 2, 3 or 4 fused or unfused rings, and is optionally substituted with one or more groups L as defined above.

A heteroaryl group as referred to above and below preferably has 4 to 30 ring C atoms, wherein one or more of the C ring atoms are replaced by a hetero atom, preferably selected from N, O, S, Si and Se, is mono- or polycyclic and may also contain fused rings, preferably contains 1, 2, 3 or 4 fused or unfused rings, and is optionally substituted with one or more groups L as defined above.

An arylalkyl or heteroarylalkyl group as referred to above and below preferably denotes —(CH₂)ₐ-aryl or —(CH₂)ₐ-heteroaryl, wherein a is an integer from 1 to 6, preferably 1, and “aryl” and “heteroaryl” have the meanings given above and below. A preferred arylalkyl group is benzyl which is optionally substituted by L.

As used herein, “arylene” will be understood to mean a divalent aryl group, and “heteroarylene” will be understood to mean a divalent heteroaryl group, including all preferred meanings of aryl and heteroaryl as given above and below.

Preferred aryl and heteroaryl groups are phenyl in which, in addition, one or more CH groups may be replaced by N, naphthalene, thiophene, selenophene, thienothiophene, dithienothiophene, fluorene and oxazole, all of which can be unsubstituted, mono- or polysubstituted with L as defined above. Very preferred rings are selected from pyrrole, preferably N-pyrrole, furan, pyridine, preferably 2- or 3-pyridine, pyrimidine, pyridazine, pyrazine, triazole, tetrazole, pyrazole, imidazole, isothiazole, thiazole, thiadiazole, isoxazole, oxazole, oxadiazole, thiophene, preferably 2-thiophene, selenophene, preferably 2-selenophene, thieno[3,2-b]thiophene, thieno[2,3-b]thiophene, furo[3,2-b]furan, furo[2,3-b]furan, seleno[3,2-b]selenophene, seleno[2,3-b]selenophene, thieno[3,2-b]selenophene, thieno[3,2-b]furan, indole, isoindole, benzo[b]furan, benzo[b]thiophene, benzo[1,2-b;4,5-b′]dithiophene, benzo[2,1-b;3,4-b′]dithiophene, quinole, 2-methylquinole, isoquinole, quinoxaline, quinazoline, benzotriazole, benzimidazole, benzothiazole, benzisothiazole, benzisoxazole, benzoxadiazole, benzoxazole, benzothiadiazole, 4H-cyclopenta[2,1-b;3,4-b′]dithiophene, 7H-3,4-dithia-7-sila-cyclopenta[a]pentalene, all of which can be unsubstituted, mono- or polysubstituted with L as defined above. Further examples of aryl and heteroaryl groups are those selected from the groups shown hereinafter.

An alkyl group or an alkoxy group, i.e., where the terminal CH₂ group is replaced by —O—, can be straight-chain or branched. It is preferably straight-chain, has 2, 3, 4, 5, 6, 7, 8, 12, 14, 16 or 18 carbon atoms and accordingly is preferably ethyl, propyl, butyl, pentyl, hexyl, heptyl, octyl, dodecyl, hexadecyl or octadecyl ethoxy, propoxy, butoxy, pentoxy, hexoxy, heptoxy, octoxy, dodecoxy or hexadecoxy, furthermore methyl, nonyl, decyl, undecyl, tridecyl, tetradecyl, pentadecyl, nonoxy, decoxy, undecoxy, tridecoxy or tetradecoxy, for example.

An alkenyl group, i.e., wherein one or more CH₂ groups are replaced by —CH═CH— can be straight-chain or branched. It is preferably straight-chain, has 2 to 10 C atoms and accordingly is preferably vinyl, prop-1-, or prop-2-enyl, but-1-, 2- or but-3-enyl, pent-1-, 2-, 3- or pent-4-enyl, hex-1-, 2-, 3-, 4- or hex-5-enyl, hept-1-, 2-, 3-, 4-, 5- or hept-6-enyl, oct-1-, 2-, 3-, 4-, 5-, 6- or oct-7-enyl, non-1-, 2-, 3-, 4-, 5-, 6-, 7- or non-8-enyl, dec-1-, 2-, 3-, 4-, 5-, 6-, 7-, 8- or dec-9-enyl.

Especially preferred alkenyl groups are C₂-C₇-1E-alkenyl, C₄-C₇-3E-alkenyl, C₅-C₇-4-alkenyl, C₆-C₇-5-alkenyl and C₇-6-alkenyl, in particular C₂-C₇-1E-alkenyl, C₄-C₇-3E-alkenyl and C₅-C₇-4-alkenyl. Examples for particularly preferred alkenyl groups are vinyl, 1E-propenyl, 1E-butenyl, 1E-pentenyl, 1E-hexenyl, 1E-heptenyl, 3-butenyl, 3E-pentenyl, 3E-hexenyl, 3E-heptenyl, 4-pentenyl, 4Z-hexenyl, 4E-hexenyl, 4Z-heptenyl, 5-hexenyl, 6-heptenyl and the like. Groups having up to 5 C atoms are generally preferred.

An oxaalkyl group, i.e., where one CH₂ group is replaced by —O—, is preferably straight-chain 2-oxapropyl (=methoxymethyl), 2-(=ethoxymethyl) or 3-oxabutyl (=2-methoxyethyl), 2-, 3-, or 4-oxapentyl, 2-, 3-, 4-, or 5-oxahexyl, 2-, 3-, 4-, 5-, or 6-oxaheptyl, 2-, 3-, 4-, 5-, 6- or 7-oxaoctyl, 2-, 3-, 4-, 5-, 6-, 7- or 8-oxanonyl or 2-, 3-, 4-, 5-, 6-, 7-, 8- or 9-oxadecyl, for example.

In an alkyl group wherein one CH₂ group is replaced by —O— and one CH₂ group is replaced by —C(O)—, these radicals are preferably neighboured. Accordingly these radicals together form a carbonyloxy group —C(O)—O— or an oxycarbonyl group —O—C(O)—. Preferably this group is straight-chain and has 2 to 6 C atoms. It is accordingly preferably acetyloxy, propionyloxy, butyryloxy, pentanoyloxy, hexanoyloxy, acetyloxymethyl, propionyloxymethyl, butyryloxymethyl, pentanoyloxymethyl, 2-acetyloxyethyl, 2-propionyloxyethyl, 2-butyryloxyethyl, 3-acetyloxypropyl, 3-propionyloxypropyl, 4-acetyloxybutyl, methoxycarbonyl, ethoxycarbonyl, propoxycarbonyl, butoxycarbonyl, pentoxycarbonyl, methoxycarbonylmethyl, ethoxycarbonylmethyl, propoxycarbonylmethyl, butoxycarbonylmethyl, 2-(methoxycarbonyl)ethyl, 2-(ethoxycarbonyl)ethyl, 2-(propoxycarbonyl)ethyl, 3-(methoxycarbonyl)propyl, 3-(ethoxycarbonyl)propyl, 4-(methoxycarbonyl)-butyl.

An alkyl group wherein two or more CH₂ groups are replaced by —O— and/or —C(O)O— can be straight-chain or branched. It is preferably straight-chain and has 3 to 12 C atoms. Accordingly it is preferably bis-carboxy-methyl, 2,2-bis-carboxy-ethyl, 3,3-bis-carboxy-propyl, 4,4-bis-carboxy-butyl, 5,5-bis-carboxy-pentyl, 6,6-bis-carboxy-hexyl, 7,7-bis-carboxy-heptyl, 8,8-bis-carboxy-octyl, 9,9-bis-carboxy-nonyl, 10,10-bis-carboxy-decyl, bis-(methoxycarbonyl)-methyl, 2,2-bis-(methoxycarbonyl)-ethyl, 3,3-bis-(methoxycarbonyl)-propyl, 4,4-bis-(methoxycarbonyl)-butyl, 5,5-bis-(methoxycarbonyl)-pentyl, 6,6-bis-(methoxycarbonyl)-hexyl, 7,7-bis-(methoxycarbonyl)-heptyl, 8,8-bis-(methoxycarbonyl)-octyl, bis-(ethoxycarbonyl)-methyl, 2,2-bis-(ethoxycarbonyl)-ethyl, 3,3-bis-(ethoxycarbonyl)-propyl, 4,4-bis-(ethoxycarbonyl)-butyl, 5,5-bis-(ethoxycarbonyl)-hexyl.

A thioalkyl group, i.e., where one CH₂ group is replaced by —S—, is preferably straight-chain thiomethyl (—SCH₃), 1-thioethyl (—SCH₂CH₃), 1-thiopropyl (═—SCH₂CH₂CH₃), 1-(thiobutyl), 1-(thiopentyl), 1-(thiohexyl), 1-(thioheptyl), 1-(thiooctyl), 1-(thiononyl), 1-(thiodecyl), 1-(thioundecyl) or 1-(thiododecyl), wherein preferably the CH₂ group adjacent to the sp² hybridised vinyl carbon atom is replaced.

A fluoroalkyl group is perfluoroalkyl CᵢF₂ᵢ₊₁, wherein i is an integer from 1 to 15, in particular CF₃, C₂F₅, C₃F₇, C₄F₉, C₅F₁₁, C₆F₁₃, C₇F₁₅ or CO₈F₁₇, very preferably C₆F₁₃, or partially fluorinated alkyl, preferably with 1 to 15 C atoms, in particular 1,1-difluoroalkyl, all of the aforementioned being straight-chain or branched.

Preferably “fluoroalkyl” means a partially fluorinated (i.e. not perfluorinated) alkyl group.

Alkyl, alkoxy, alkenyl, oxaalkyl, thioalkyl, carbonyl and carbonyloxy groups can be achiral or chiral groups. Particularly preferred chiral groups are 2-butyl (=1-methylpropyl), 2-methylbutyl, 2-methylpentyl, 3-methylpentyl, 2-ethylhexyl, 2-butyloctyl, 2-hexyldecyl, 2-octyldodecyl, 2-propylpentyl, in particular 2-methylbutyl, 2-methylbutoxy, 2-methylpentoxy, 3-methylpentoxy, 2-ethyl-hexoxy, 2-butyloctoxyo, 2-hexyldecoxy, 2-octyldodecoxy, 1-methylhexoxy, 2-octyloxy, 2-oxa-3-methylbutyl, 3-oxa-4-methyl-pentyl, 4-methylhexyl, 2-hexyl, 2-octyl, 2-nonyl, 2-decyl, 2-dodecyl, 6-methoxy-octoxy, 6-methyloctoxy, 6-methyloctanoyloxy, 5-methylheptyloxy-carbonyl, 2-methylbutyryloxy, 3-methylvaleroyloxy, 4-methylhexanoyloxy, 2-chloro-propionyloxy, 2-chloro-3-methylbutyryloxy, 2-chloro-4-methyl-valeryl-oxy, 2-chloro-3-methylvaleryloxy, 2-methyl-3-oxapentyl, 2-methyl-3-oxa-hexyl, 1-methoxypropyl-2-oxy, 1-ethoxypropyl-2-oxy, 1-propoxypropyl-2-oxy, 1-butoxypropyl-2-oxy, 2-fluorooctyloxy, 2-fluorodecyloxy, 1,1,1-trifluoro-2-octyloxy, 1,1,1-trifluoro-2-octyl, 2-fluoromethyloctyloxy for example. Very preferred are 2-ethylhexyl, 2-butyloctyl, 2-hexyldecyl, 2-octyldodecyl, 2-hexyl, 2-octyl, 2-octyloxy, 1,1,1-trifluoro-2-hexyl, 1,1,1-trifluoro-2-octyl and 1,1,1-trifluoro-2-octyloxy.

Preferred achiral branched groups are isopropyl, isobutyl (=methylpropyl), isopentyl (=3-methylbutyl), tert. butyl, isopropoxy, 2-methyl-propoxy and 3-methylbutoxy.

In a preferred embodiment the substituents on the aryl and heteroaryl rings are independently of each other selected from primary, secondary or tertiary alkyl or alkoxy with 1 to 30 C atoms, wherein one or more H atoms are optionally replaced by F, or aryl, aryloxy, heteroaryl or heteroaryloxy that is optionally alkylated or alkoxylated and has 4 to 30 ring atoms. Very preferred substituents are selected from the group consisting of the following formulae

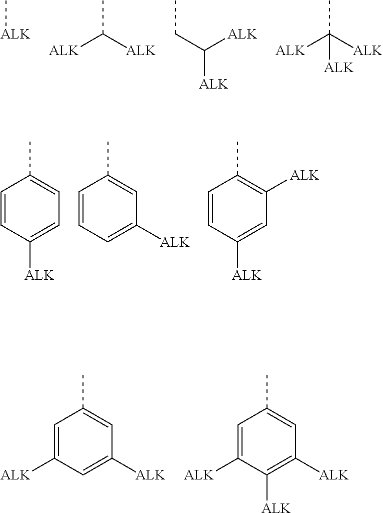

wherein “ALK” denotes optionally fluorinated, preferably linear, alkyl or alkoxy with 1 to 20, preferably 1 to 16 C-atoms, in case of tertiary groups very preferably 1 to 9 C atoms, and the dashed line denotes the link to the ring to which these groups are attached. Especially preferred among these groups are those wherein all ALK subgroups are identical.

As used herein, if an aryl(oxy) or heteroaryl(oxy) group is “alkylated or alkoxylated”, this means that it is substituted with one or more alkyl or alkoxy groups having from 1 to 20 C-atoms and being straight-chain or branched and wherein one or more H atoms are optionally substituted by an F atom.

Above and below, Y¹ and Y² are independently of each other H, F, Cl or CN.

As used herein, —CO—, —C(═O)— and —C(O)— will be understood to mean a carbonyl group, i.e. a group having the structure

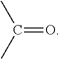

As used herein, C═CR¹R² etc. will be understood to mean a group having the structure

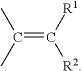

Unless stated otherwise “optionally substituted” without mentioning the substitutent means optionally substituted by L.

As used herein, “halogen” includes F, Cl, Br or I, preferably F, Cl or Br. A halogen atom that represents a substituent on a ring or chain is preferably F or Cl, very preferably F. A halogen atom that represents a reactive group in a monomer is preferably Cl, Br or I, very preferably Br or I.

Above and below, “mirror image” means a moiety that is obtainable from another moiety by flipping it vertically or horizontally across an external symmetry plane or a symmetry plane extending through the moiety. For example the moiety

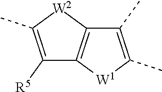

also includes the mirror images

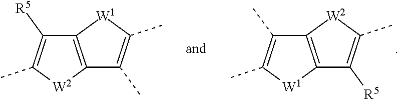

### DETAILED DESCRIPTION

The compounds of the present invention are easy to synthesize and exhibit advantageous properties. They show good processability for the device manufacture process, high solubility in organic solvents, and are especially suitable for large scale production using solution processing methods.

Co-polymers derived from monomers of the present invention and electron acceptor monomers show low bandgaps, high charge carrier mobilities, high external quantum efficiencies in BHJ solar cells, good morphology when used in p/n-type blends e.g. with fullerenes, high oxidative stability, a long lifetime in electronic devices, and are promising materials for organic electronic OE devices, especially for OPV devices with high power conversion efficiency.

The compounds of the present invention are especially suitable as both p-type and n-type semiconductors, depending on the nature of the co-monomer or n-units cross-coupled to extend the conjugation, for the preparation of blends of p-type and n-type semiconductors which are suitable for use in BHJ photovoltaic devices.

Besides, the compounds of the present invention show the following advantageous properties:
i) The fused dithiole rings are expected to improve the stacking of the molecules or polymer backbones due to the well established S—S interactions.
ii) The strong electron deficient nature of the dicyanomethylene functional group will offset the electron donating property of the dithiolo sulfur atoms, so that the whole unit remains as an electron acceptor unit.
iii) The units are basic versatile aromatic systems that allow a variety of derivatisations through well-established cross-coupling reactions.

In the units of formula I and its subformulae X is preferably S, O, NR or —CR¹═CR²—, very preferably S, O, NH or —CR¹═CR²—, most preferably S or —CR¹═CR²—.

In the units of formula I and its subformulae preferably U¹ and U² denote an electron withdrawing group selected from CN, C(═O)R and C(═O)OR.

Preferably R¹ and R² in formula I are selected from the following groups or any combination thereof:
the group consisting of R, —OR and —SR wherein R is straight-chain or branched alkyl with 1 to 25, preferably 1 to 18 C atoms that is optionally fluorinated,
the group consisting of —C(═O)—R, —C(═O)—OR, —OC(═O)—R, —C(═O)—NHR and —C(═O)—NRRⁿ, wherein R and Rⁿ are independently of each other straight-chain or branched alkyl with 1 to 25, preferably 1 to 18 C atoms that is optionally fluorinated,
the group consisting of aryl, aryloxy, heteroaryl and heteroaryloxy, each of which has 5 to 20 ring atoms and optionally contains fused rings and is unsubstituted or substituted by one or more groups L as defined in formula I,
the group consisting of F, Cl and CN, very preferably F.

In another preferred embodiment R¹ and R² form an aromatic or heteroaromatic ring system that is fused to the pyrazine ring to which R¹ and R² are attached, has 5 to 20 ring atoms, is mono- or polycyclic, optionally contains fused rings, and is unsubstituted or substituted by one or more identical or different groups L.

Preferably R in formula I denotes straight-chain or branched alkyl with 1 to 25, very preferably 1 to 18 C atoms, which is optionally fluorinated.

Further preferably R in formula I is selected from the group consisting of aryl, aryloxy, heteroaryl and heteroaryloxy, each of which has 5 to 20 ring atoms and optionally contains fused rings and is unsubstituted or substituted by one or more groups L as defined in formula I.

If R^{1,2} or R denote an aryl(oxy) or heteroaryl(oxy) group, it is preferably selected from phenyl, pyrrole, furan, pyridine, thiazole, thiophene, thiadiazole, triazole, pyrazine, thieno[3,2-b]thiophene or thieno[2,3-b]thiophene, each of which is unsubstituted or substituted with F or alkyl, alkoxy or thioalkyl having 1 to 20 C atoms and being optionally fluorinated.

In another preferred embodiment of the present invention one or more of R¹, R² and R denote straight-chain, branched or cyclic alkyl with 1 to 20 C-atoms wherein one or more CH₂ or CH₃ groups are substituted by a cationic or anionic group.

The cationic group is preferably selected from the group consisting of phosphonium, sulfonium, ammonium, uronium, thiouronium, guanidinium or heterocyclic cations such as imidazolium, pyridinium, pyrrolidinium, triazolium, morpholinium or piperidinium cation.

Preferred cationic groups are selected from the group consisting of tetraalkylammonium, tetraalkylphosphonium, N-alkylpyridinium, N,N-dialkylpyrrolidinium, 1,3-dialkylimidazolium, wherein “alkyl” preferably denotes a straight-chain or branched alkyl group with 1 to 12 C atoms.

Further preferred cationic groups are selected from the group consisting of the following formulae

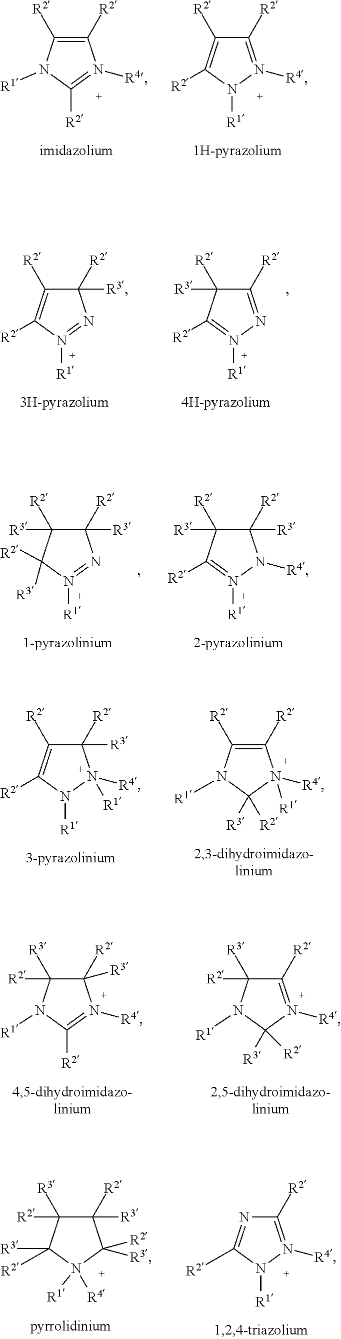

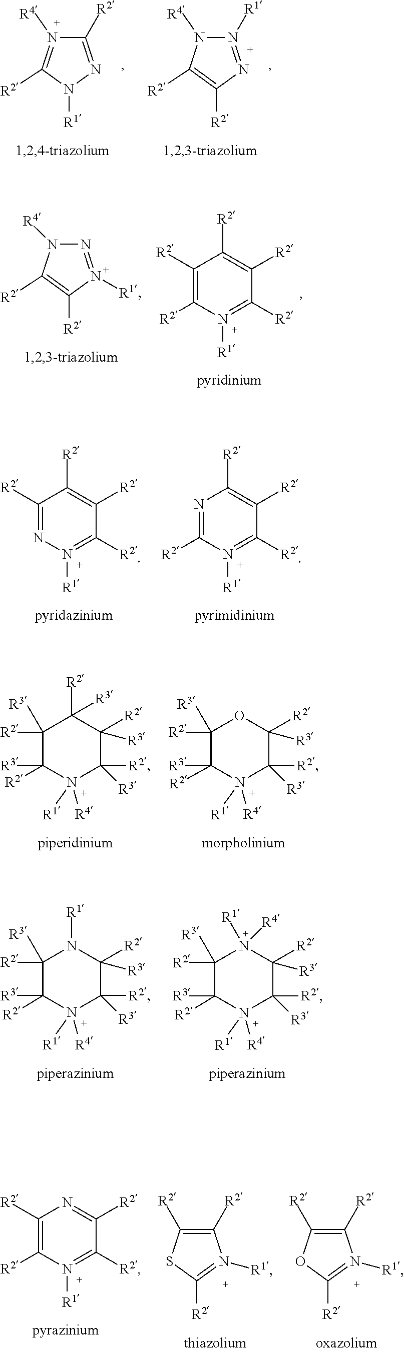

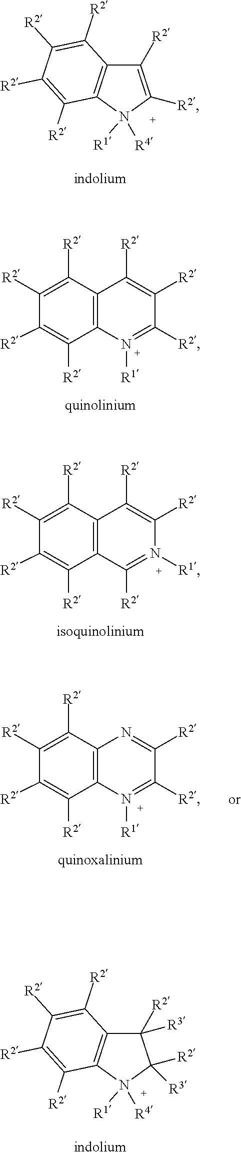

wherein R¹′, R²′, R³′ and R⁴′ denote, independently of each other, H, a straight-chain or branched alkyl group with 1 to 12 C atoms or non-aromatic carbo- or heterocyclic group or an aryl or heteroaryl group, each of the aforementioned groups having 3 to 20, preferably 5 to 15, ring atoms, being mono- or polycyclic, and optionally being substituted by one or more identical or different substituents L as defined below, or denote a link to the respective group R or R¹⁻².

In the above cationic groups of the above-mentioned formulae any one of the groups R¹′, R²′, R³′ and R⁴′ (if they replace a CH₃ group) can denote a link to the group R¹, or two neighbored groups R¹′, R²′, R³′ or R⁴′ (if they replace a CH₂ group) can denote a link to the respective group R or R¹⁻².

The anionic group is preferably selected from the group consisting of borate, imide, phosphate, sulfonate, sulfate, succinate, naphthenate or carboxylate, very preferably from phosphate, sulfonate or carboxylate.

The compounds according to the present invention include small molecules, monomers, oligomers and polymers.

A preferred embodiment of the present invention relates to a conjugated polymer comprising, preferably consisting of, one or more repeating units of formula II1 and/or II2, and optionally one or more repeating units of formula II3:

—(Ar¹)ₐ—U—(Ar²)_{b}—(Ar³)_{c}—(Ar⁴)_{d}— II1

—(Ar¹)ₐ—(Ar²)_{b}—U—(Ar³)_{c}—(Ar⁴)_{d}— II2

—(Ar¹)ₐ—(Ar²)_{b}—(Ar³)_{c}—(Ar⁴)_{d}— II3

wherein the individual radicals, independently of each other and on each occurrence identically or differently, have the following meanings
U a unit of formula I or its subformulae as defined above and below,
Ar¹⁻⁴ arylene or heteroarylene that has 5 to 20 ring atoms, is mono- or polycyclic, does optionally contain fused rings, is unsubstituted or substituted by one or more identical or different groups L, R¹ or R² as defined in formula I, and is different from U,
a, b, c, d 0 or 1, wherein in formula II3 a+b+c+d≥1.

Preferably the conjugated polymer comprises one or more repeating units of formula II1 or II2 wherein a+b+c+d≥1.

Further preferably the conjugated polymer comprises one or more repeating units of formula II1 wherein b=1 and a=c=d=0 and one or more repeating units of formula II3 wherein a=b=0 and c=d=1.

Further preferably the conjugated polymer comprises two or more distinct repeating units of formula Ill wherein b=1 and a=c=d=0.

Further preferably at least one of Ar¹, Ar², Ar³ and Ar⁴ is an arylene or heteroarylene group as being defined in formula II1 and having electron donor property.

Preferably L denotes F or is selected from the following groups
the group consisting of R, —OR and —SR wherein R is straight-chain or branched alkyl with 1 to 25, preferably 1 to 18 C atoms which is optionally fluorinated,
the group consisting of —C(═O)—R, —C(═O)—OR, —OC(═O)—R, —C(═O)—NHR and —C(═O)—NRRⁿ, wherein R and Rⁿ are independently of each other straight-chain or branched alkyl with 1 to 25, preferably 1 to 18 C atoms that is optionally fluorinated.

Further preferably the conjugated polymer according to the present invention is selected of formula III:

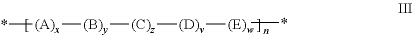

wherein the individual radicals, independently of each other and on each occurrence identically or differently, have the following meanings
A a unit of formula I, II1 or II2 as defined above and below,
B, C, D, E a unit of formula I, II1, II2 or II3 as defined above and below,
x >0 and ≤1,
v, w, y, z ≥0 and <1,
v+w+x+y+z 1, and
n an integer >1, preferably ≥5.

In the polymers of formula III and its subformulae, v, w, x, y and z denote the mole fraction of repeating units A and B, respectively, and n denotes the degree of polymerisation or total number of repeating units A and B. These formulae include block copolymers, random or statistical copolymers and alternating copolymers of A and B, as well as homopolymers of A for the case when x>0 and v=w=y=z=0.

In the polymers of formula III and its subformulae wherein one of v, w, y and z is not 0 and the others of v, w, y and z are 0, x and the one of v, w, y and z which is not 0 are each preferably from 0.1 to 0.9, very preferably from 0.3 to 0.7.

In the polymers of formula III and its subformulae wherein two of v, w, y and z are not 0 and the others of v, w, y and z are 0, x and those of v, w, y and z which are not 0 are each preferably from 0.1 to 0.8, very preferably from 0.2 to 0.6.

In the polymers of formula III and its subformulae wherein three of v, w, y and z are not 0 and the others of v, w, y and z are 0, x and those of v, w, y and z which are not 0 are each preferably from 0.1 to 0.7, very preferably from 0.2 to 0.5.

In the polymers of formula III and its subformulae wherein all of v, w, y and z are not 0, x, v, w, y and z are each preferably from 0.1 to 0.6, very preferably from 0.2 to 0.4.

In the polymers according to the present invention, the total number of repeating units n is preferably from 2 to 10,000. The total number of repeating units n is preferably ≥5, very preferably ≥10, most preferably ≥50, and preferably ≤500, very preferably ≤1,000, most preferably ≤2,000, including any combination of the aforementioned lower and upper limits of n.

The polymers of the present invention include homopolymers and copolymers, like statistical or random copolymers, alternating copolymers and block copolymers, as well as combinations thereof.

Preferred polymers of formula III are selected from the following subformulae

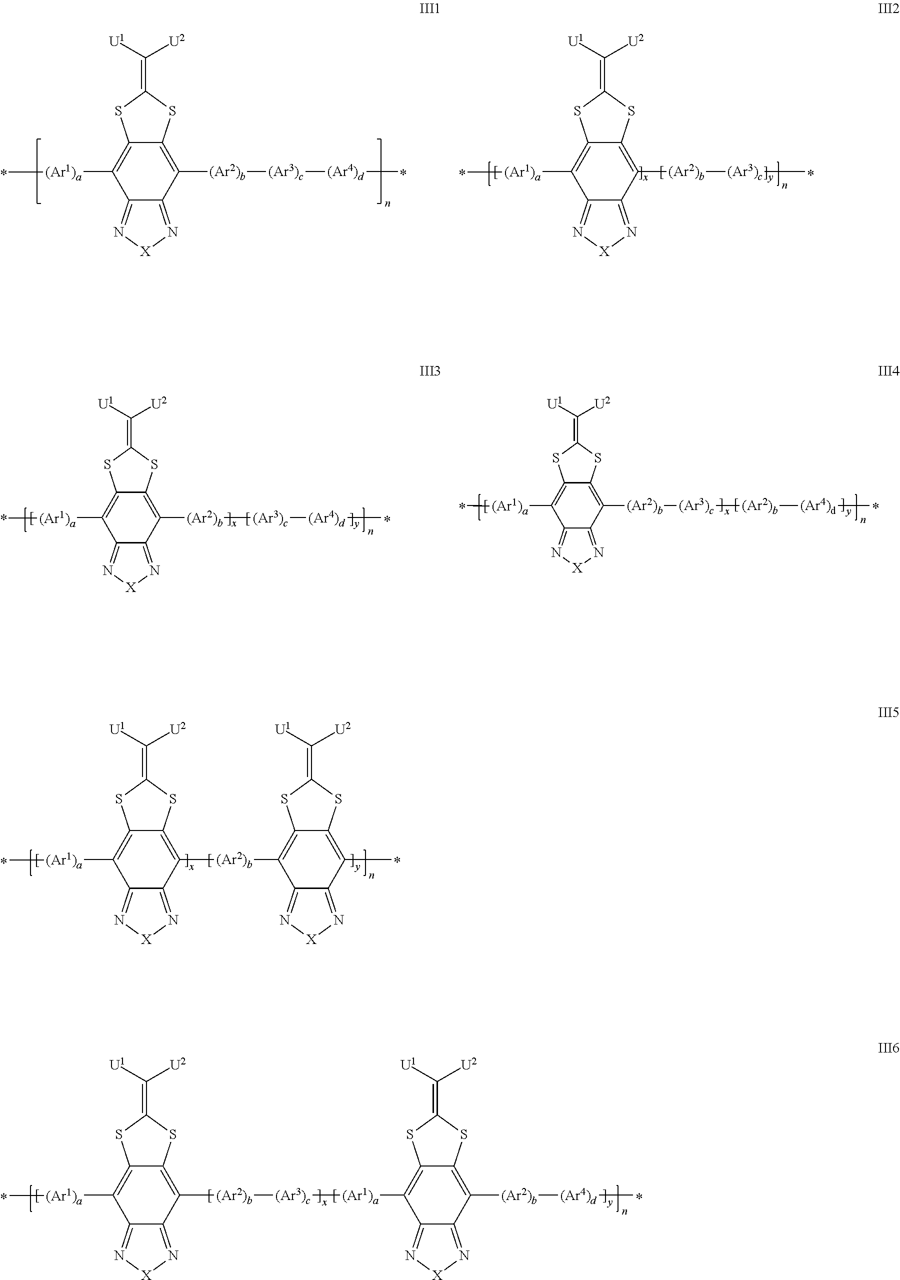

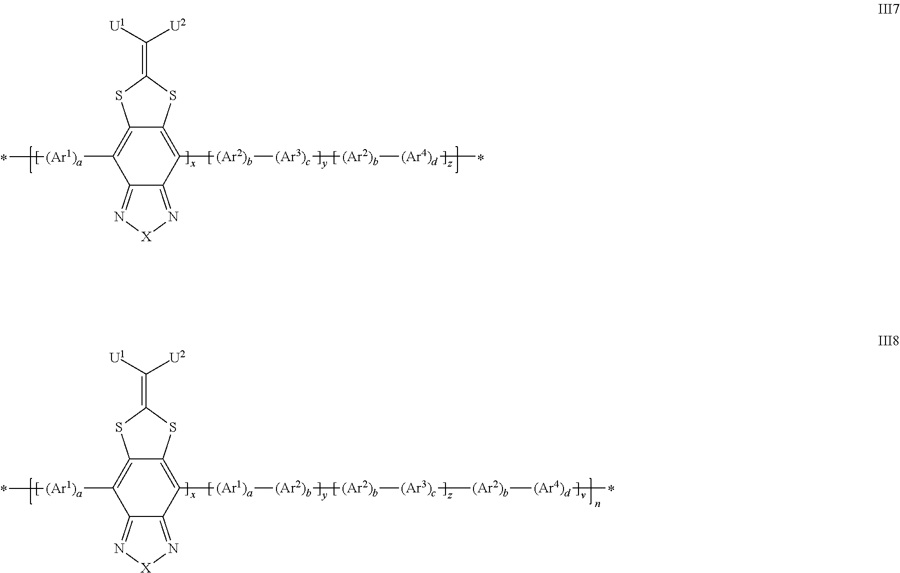

wherein X, U¹, U², Ar¹, Ar², Ar³, Ar⁴, a, b, c, d, v, x, y, z and n have the meanings of formula I, II1 and III or one of the preferred meanings given above and below, and preferably one or more of Ar³ and Ar⁴ are selected from arylene or heteroarylene units as described above and below having electron donor properties.

Especially preferred are repeating units and polymers of formulae II1, II2, II3, III, III1-III8 and their subformulae wherein one or more of Ar¹, Ar², Ar³ and Ar⁴ denote arylene or heteroarylene, preferably having electron donor properties, selected from the group consisting of the following formulae

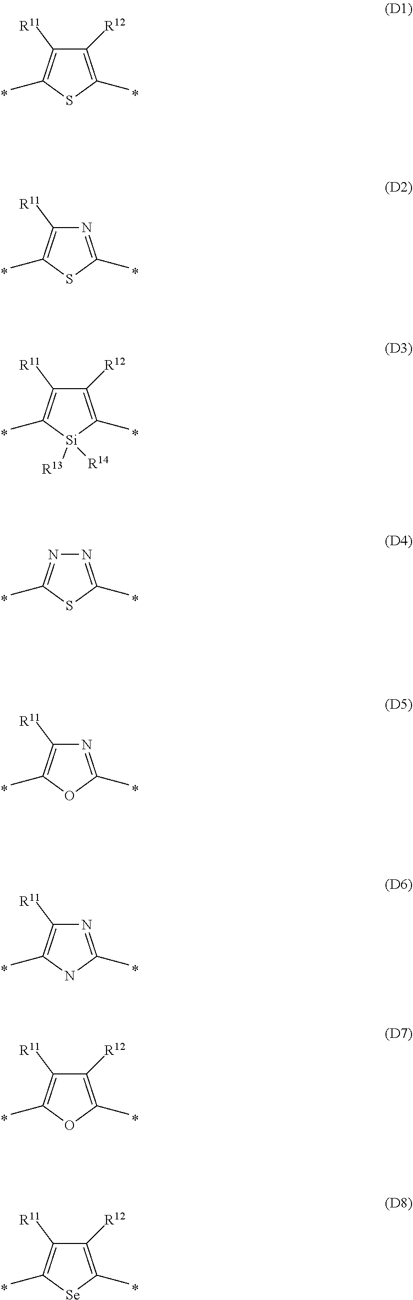

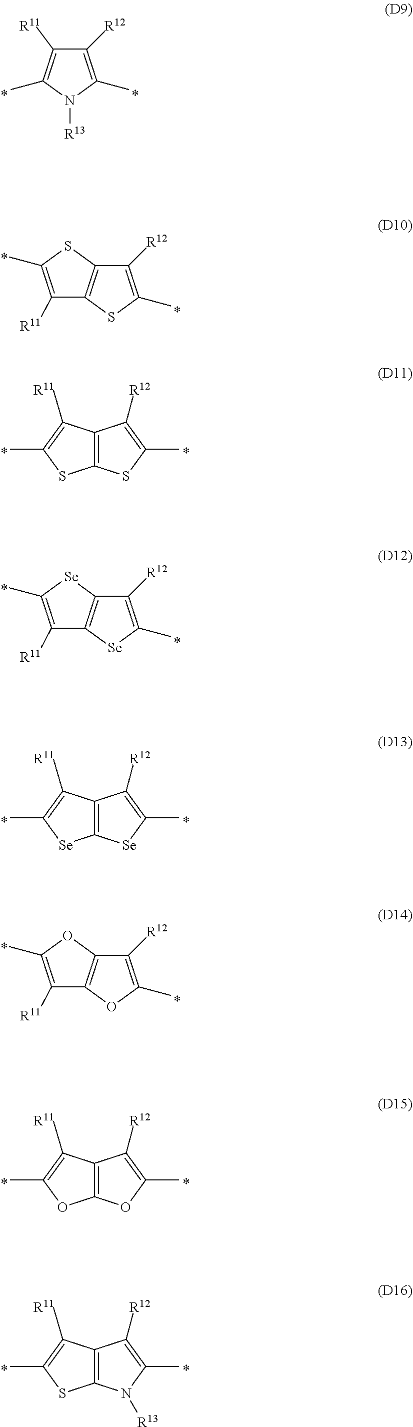

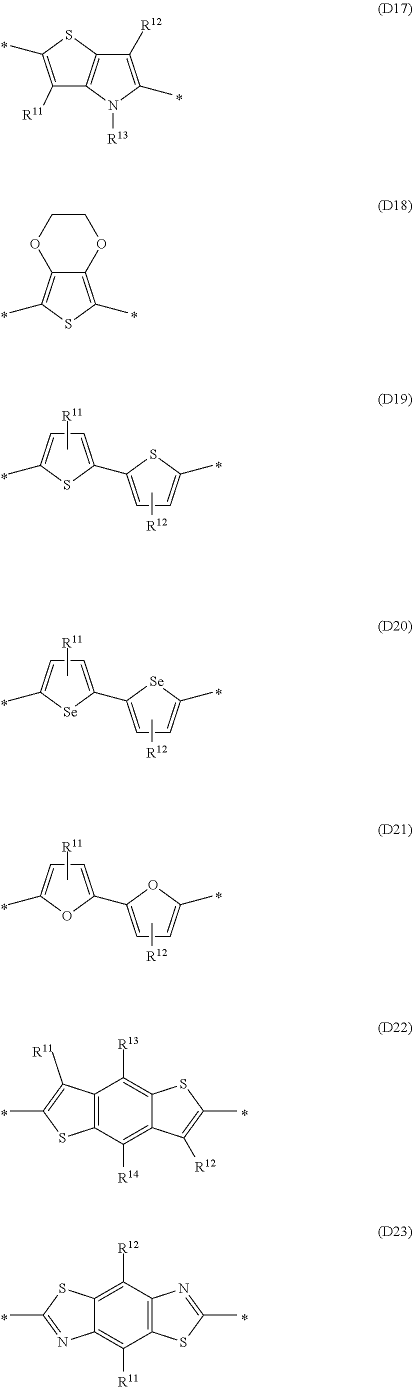

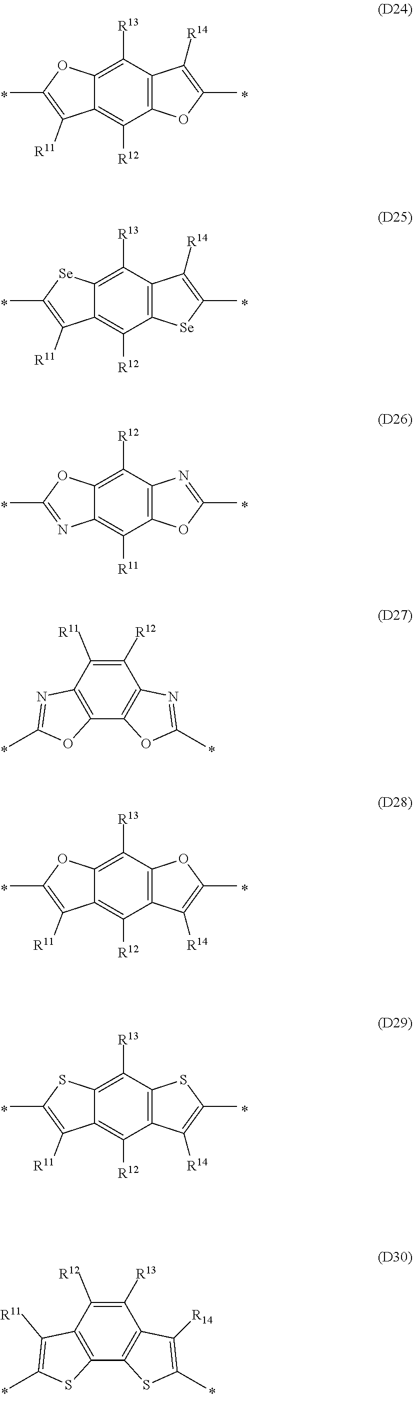

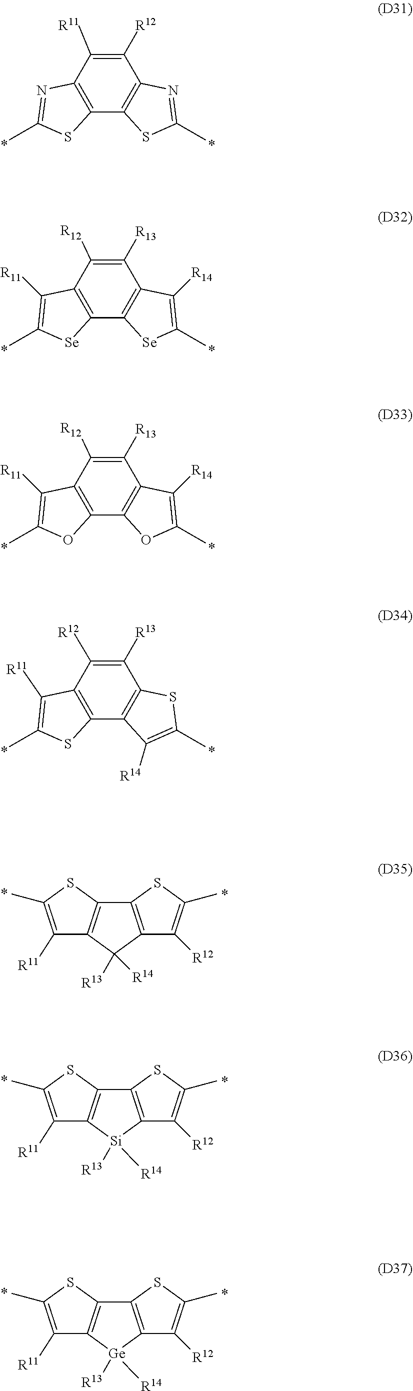

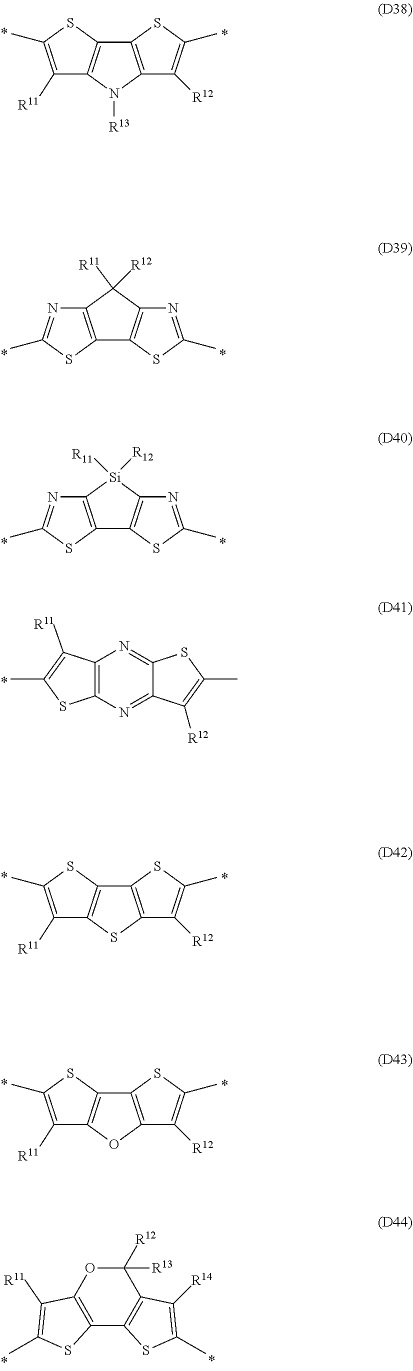

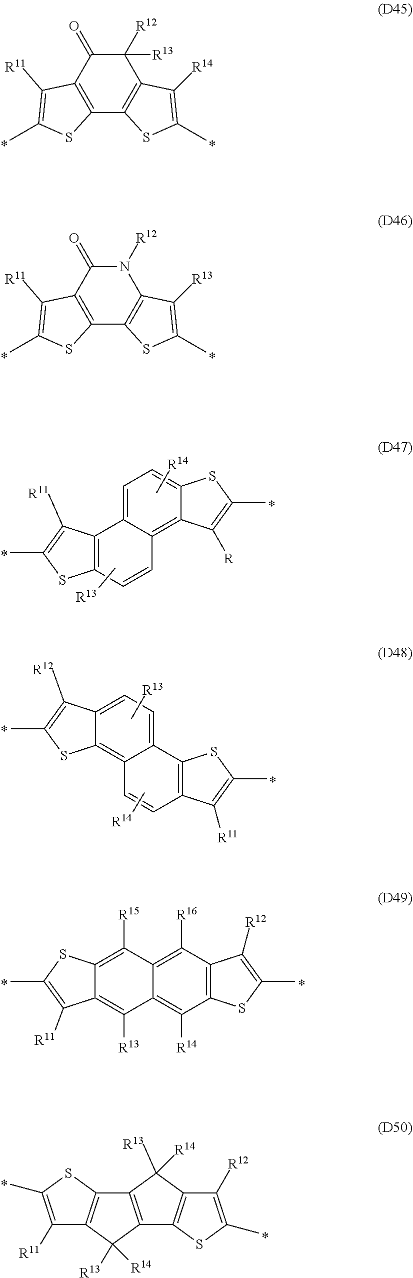

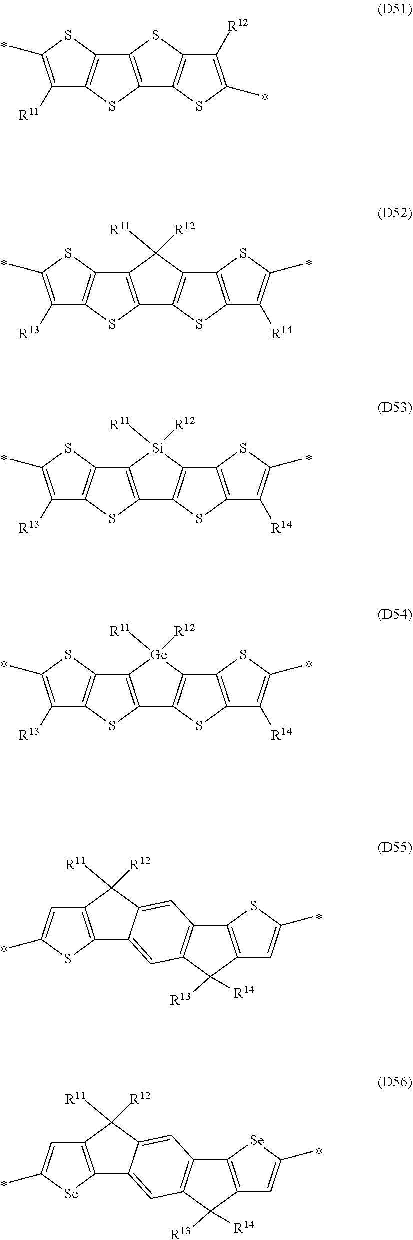

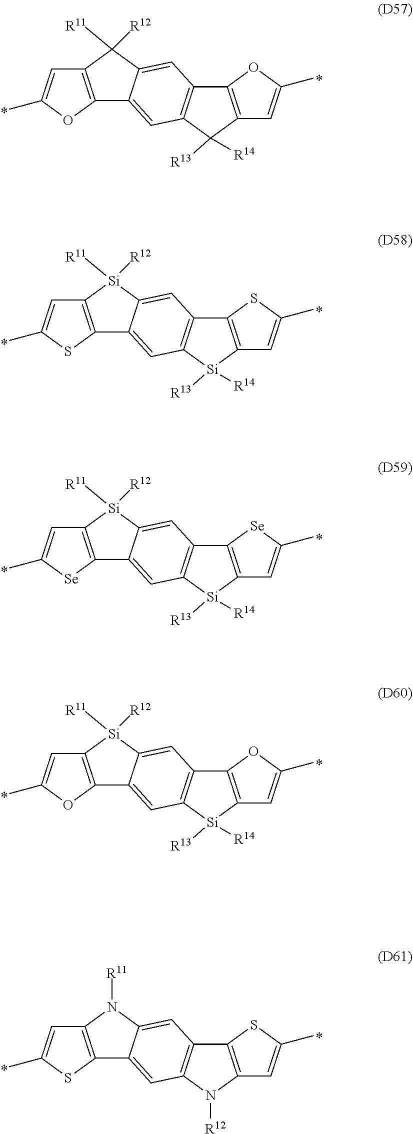

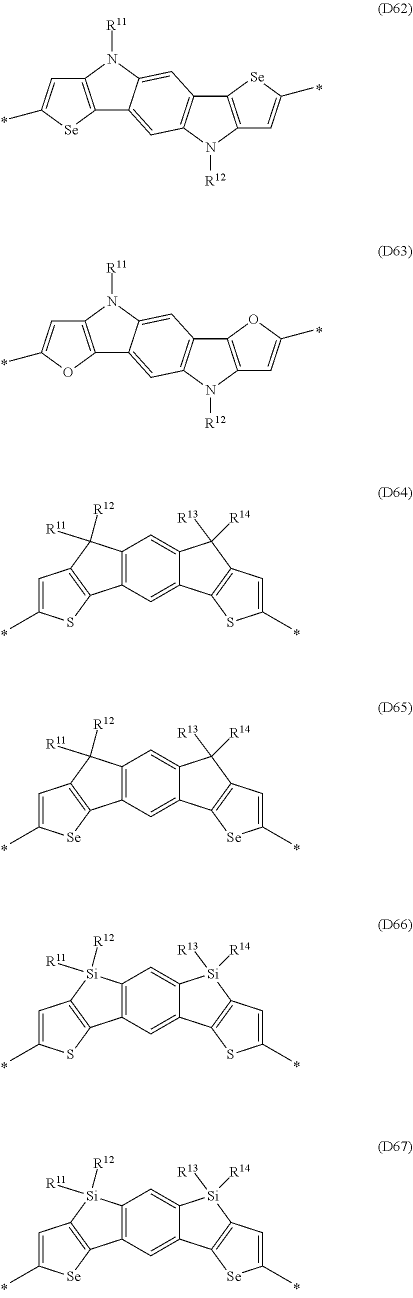

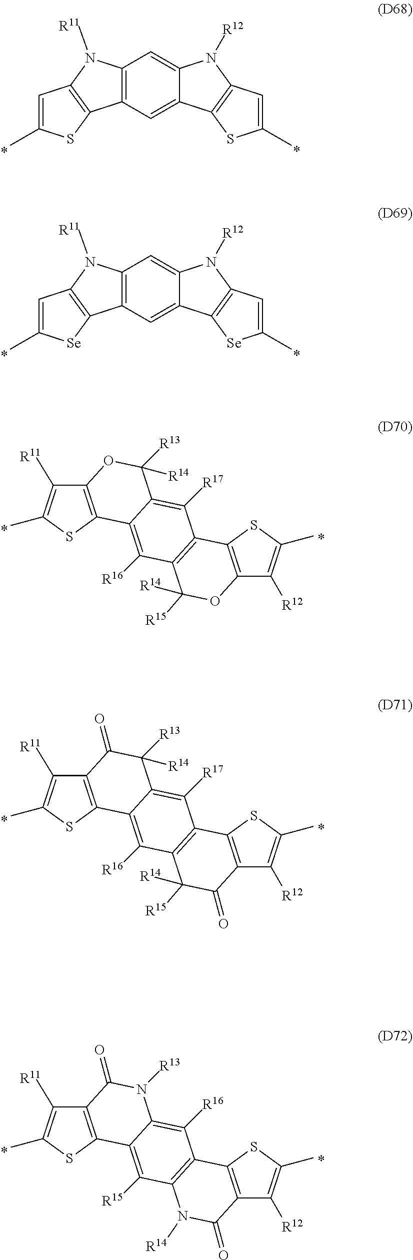

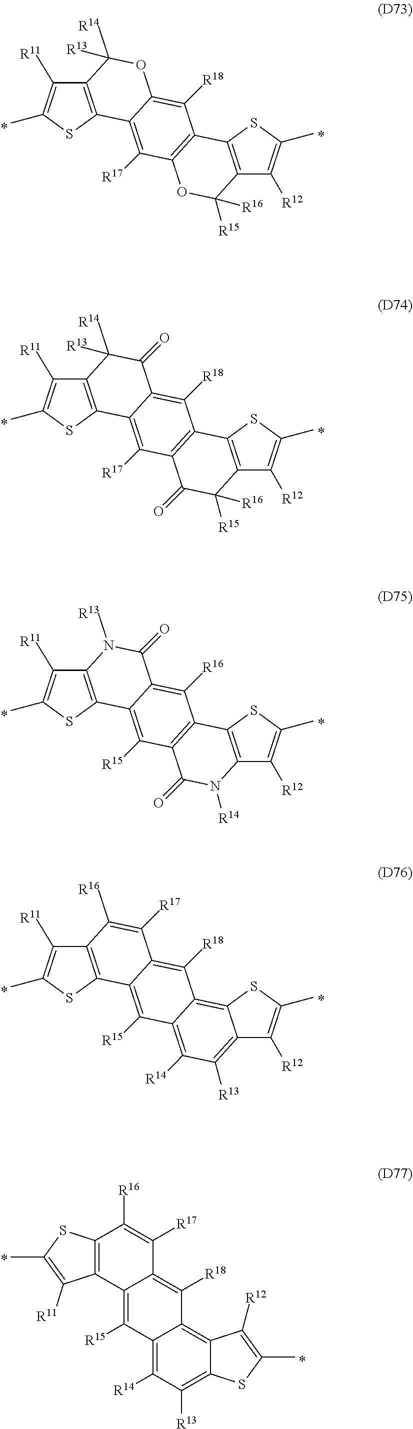

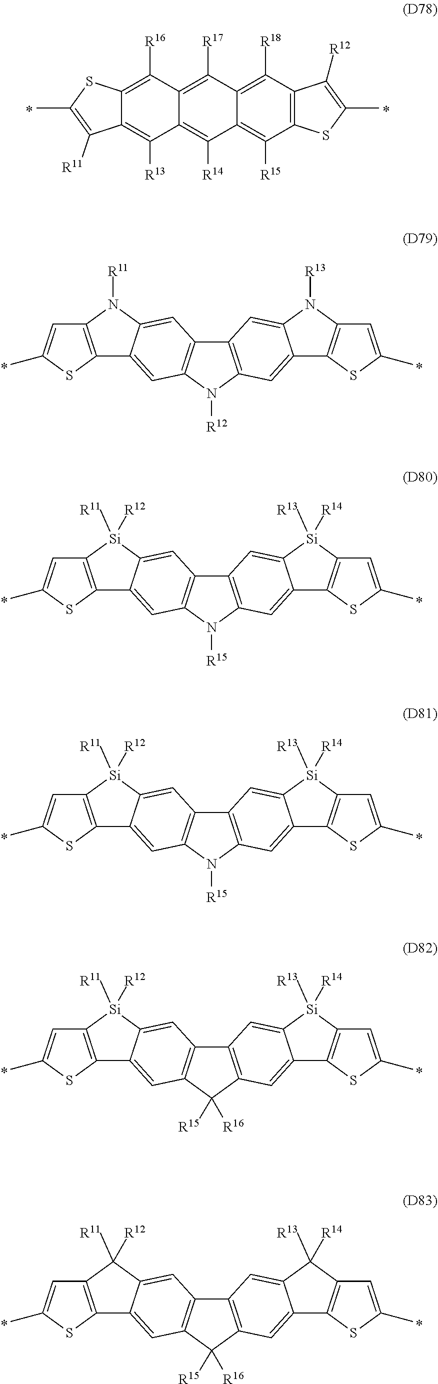

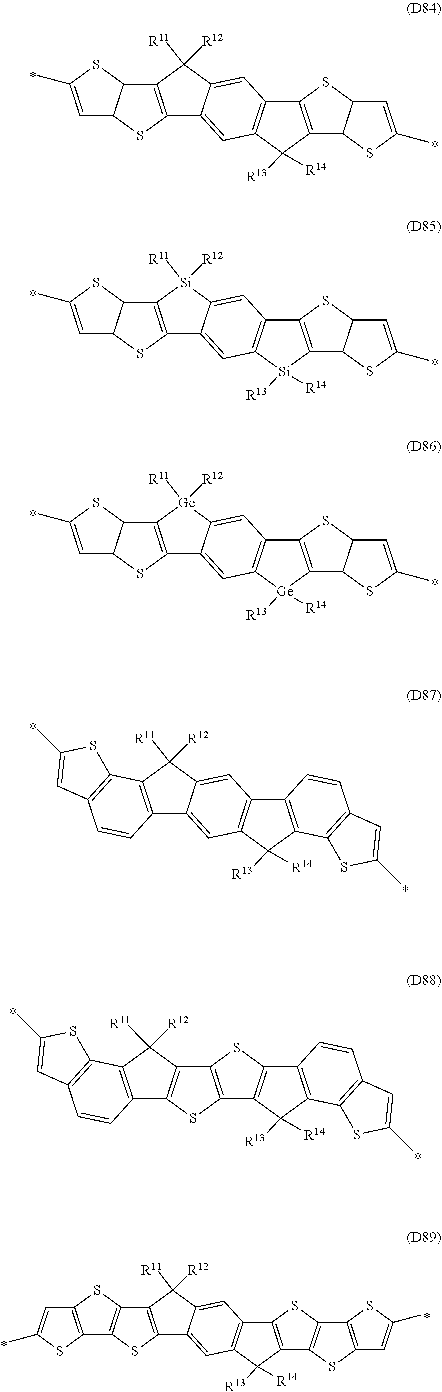

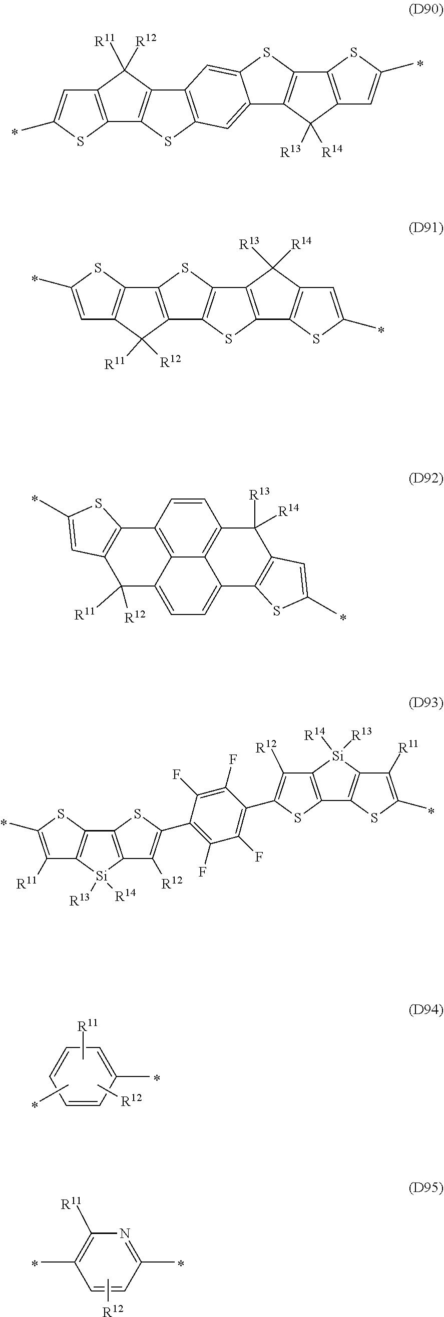

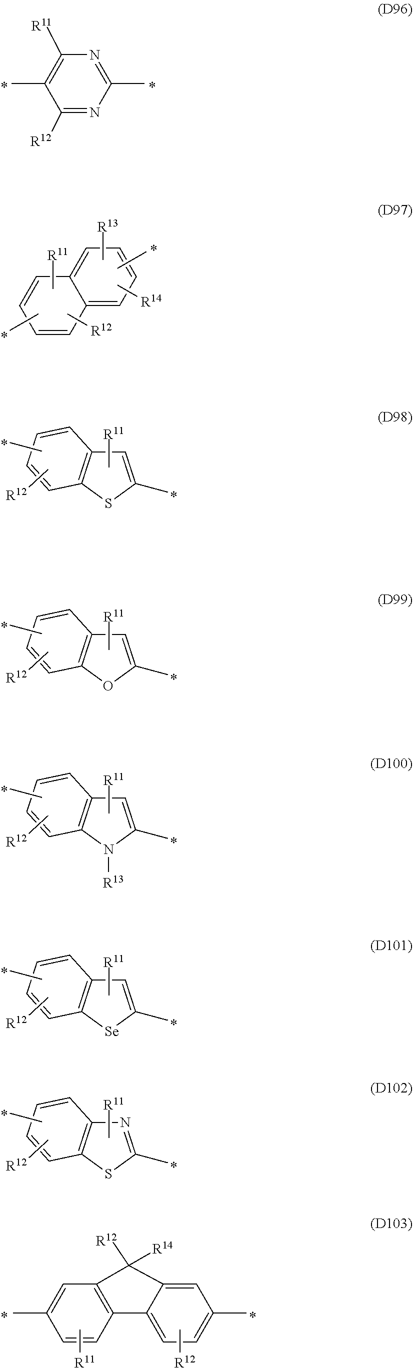

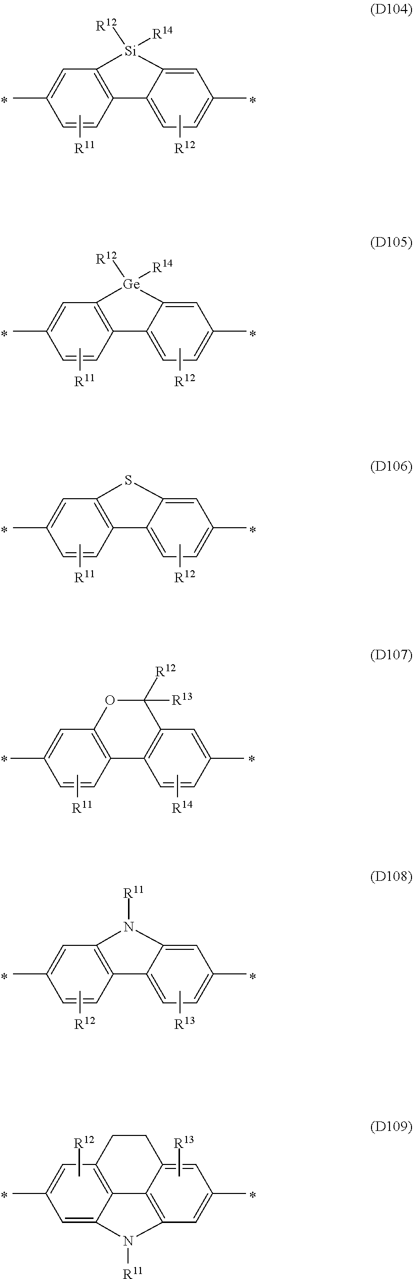

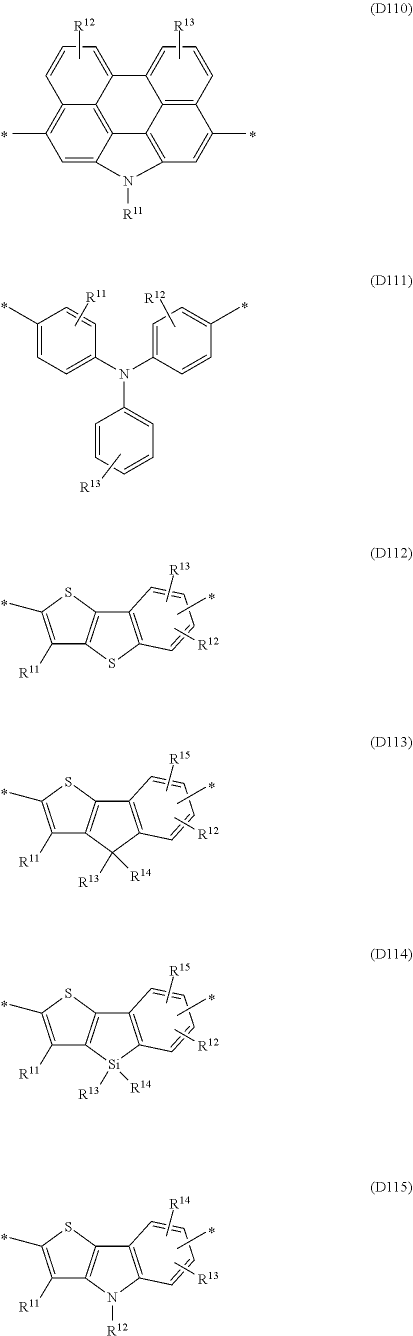

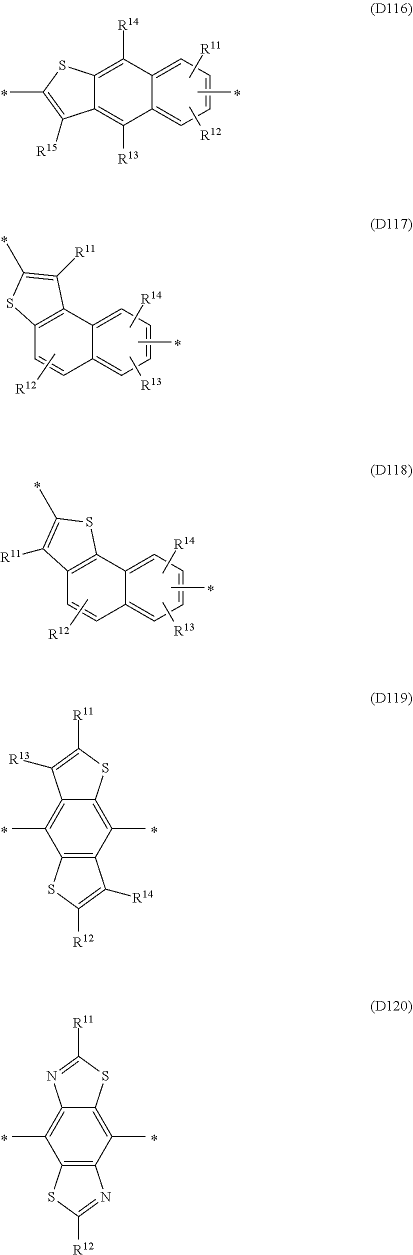

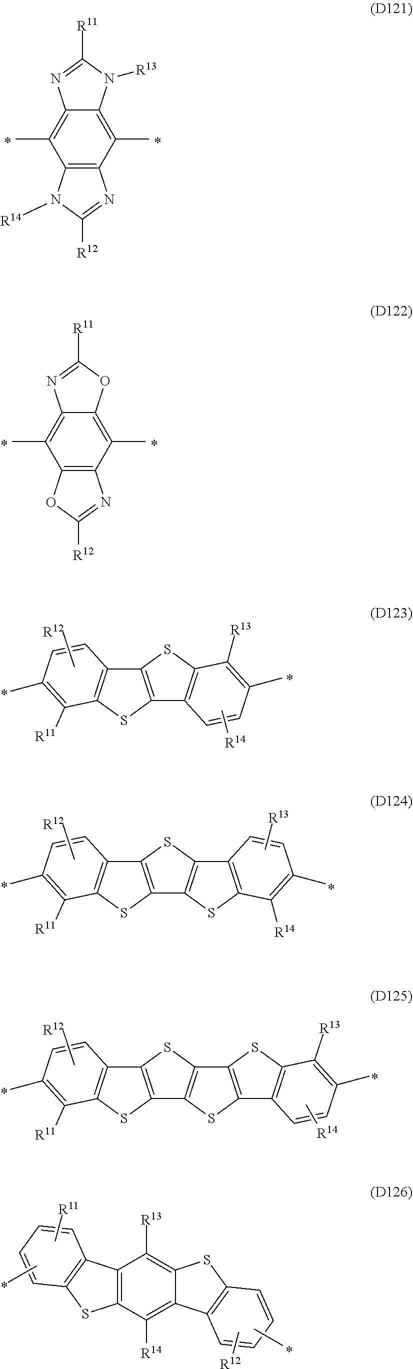

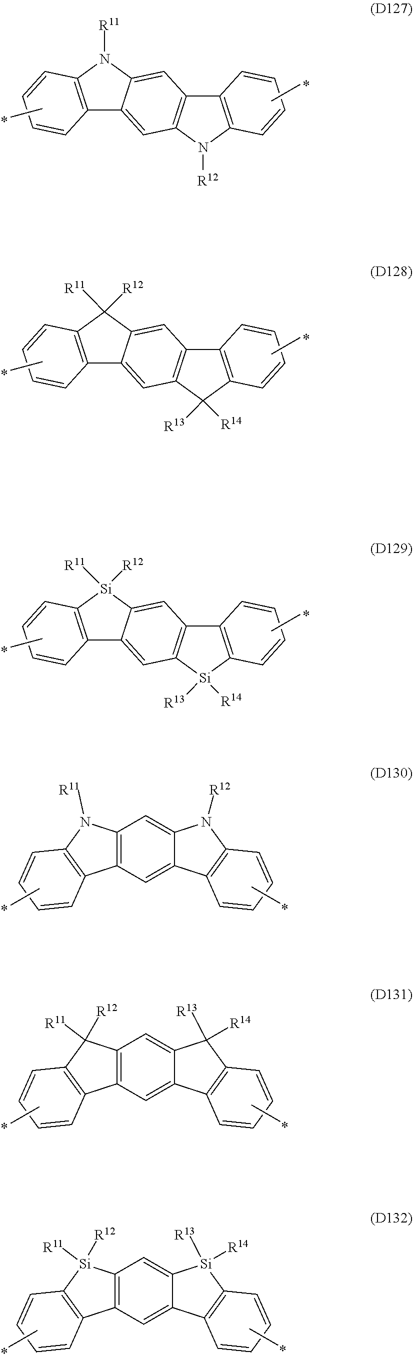

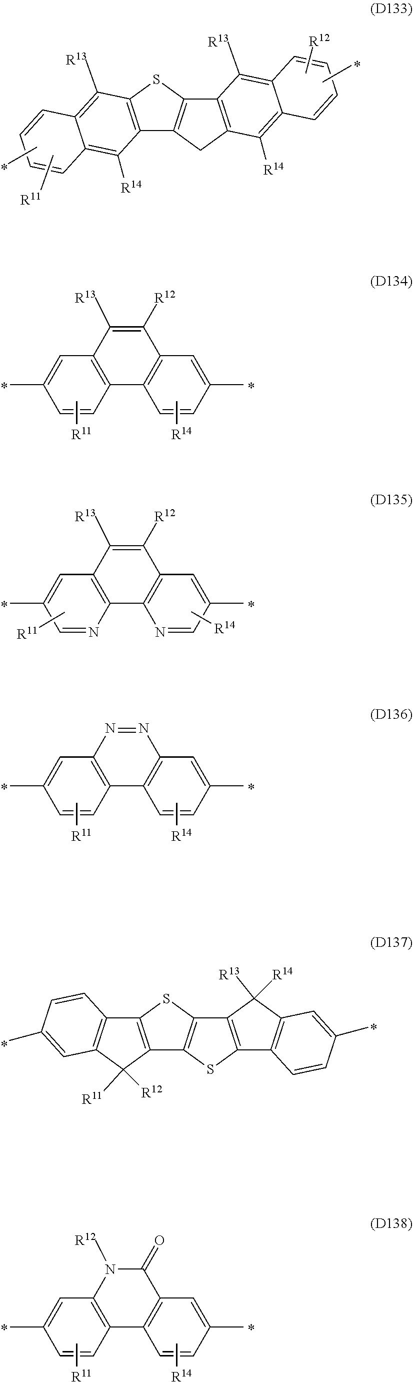

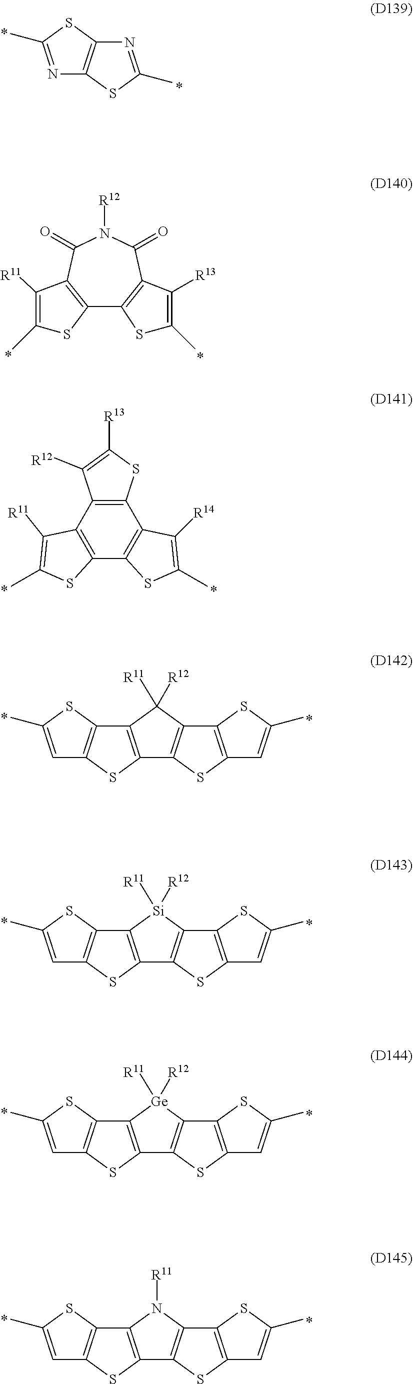

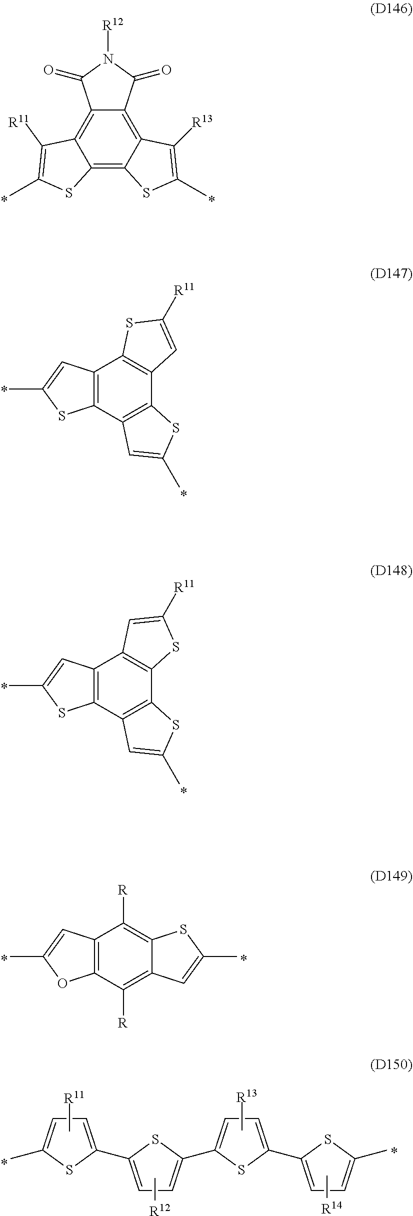

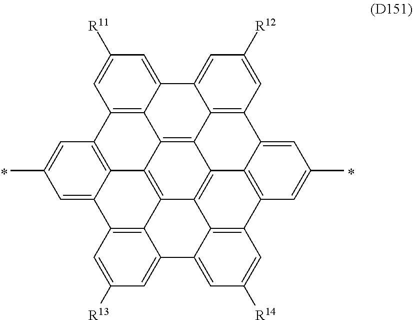

wherein R¹¹, R¹², R¹³, R¹⁴, R¹⁵, R¹⁶, R¹⁷ and R¹⁸ independently of each other denote H or have one of the meanings of L, R¹ or R² as defined above and below.

Preferred donor units are selected from formulae D1, D7, D10, D11, D19, D22, D29, D30, D35, D36, D37, D44, D55, D84, D87, D88, D89, D93, D94, D106, D111, D139, D140, D141, D146 or D150 wherein preferably at least one of R¹, R¹², R¹³ and R¹⁴ is different from H.

Further preferred are repeating units and polymers of formulae II1, II2, II3, III, III1-III8 and their subformulae wherein one or more of Ar¹, Ar², Ar³ and Ar⁴ denote arylene or heteroarylene, preferably having electron acceptor properties, selected from the group consisting of the following formulae

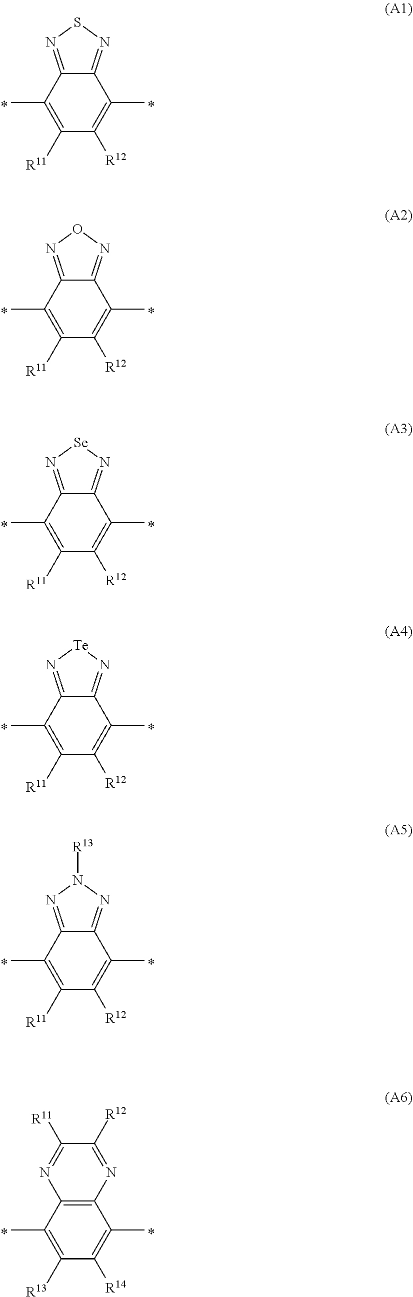

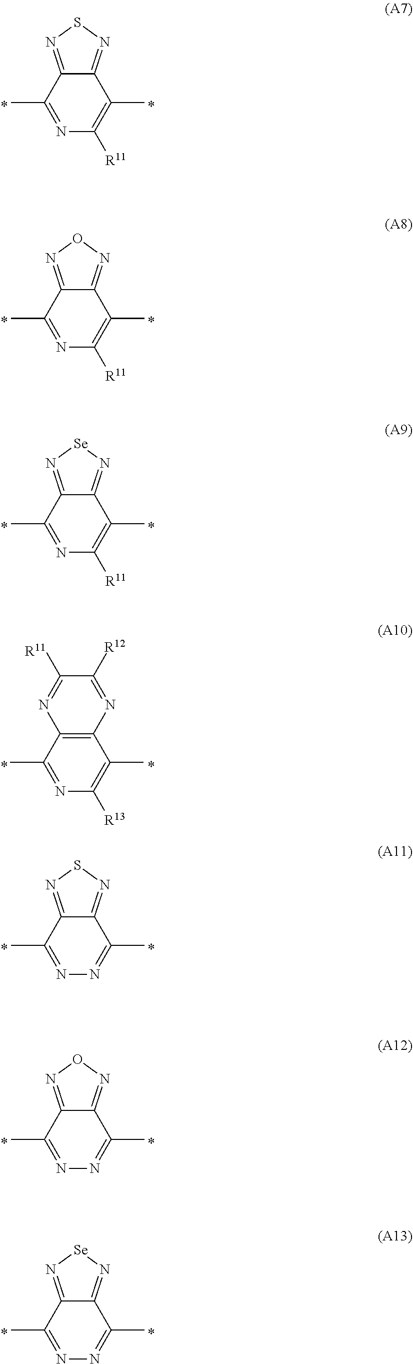

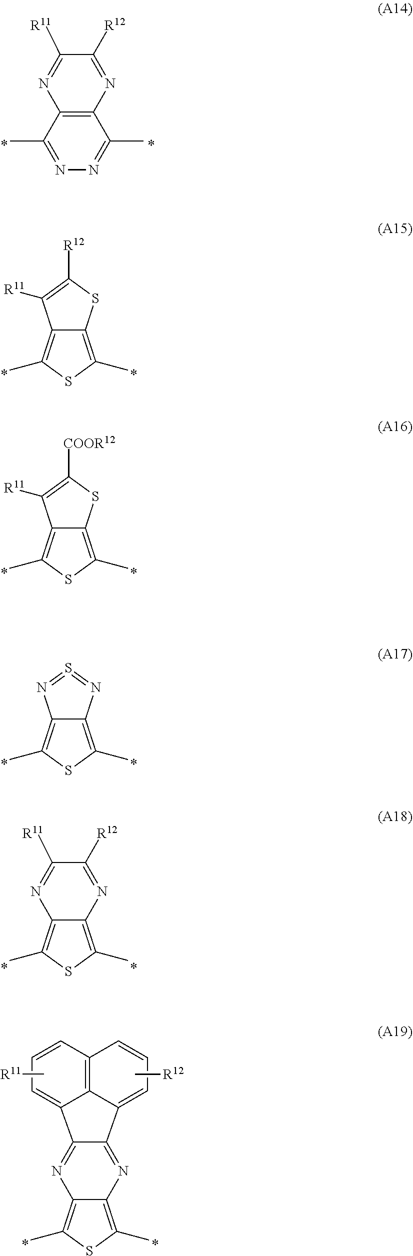

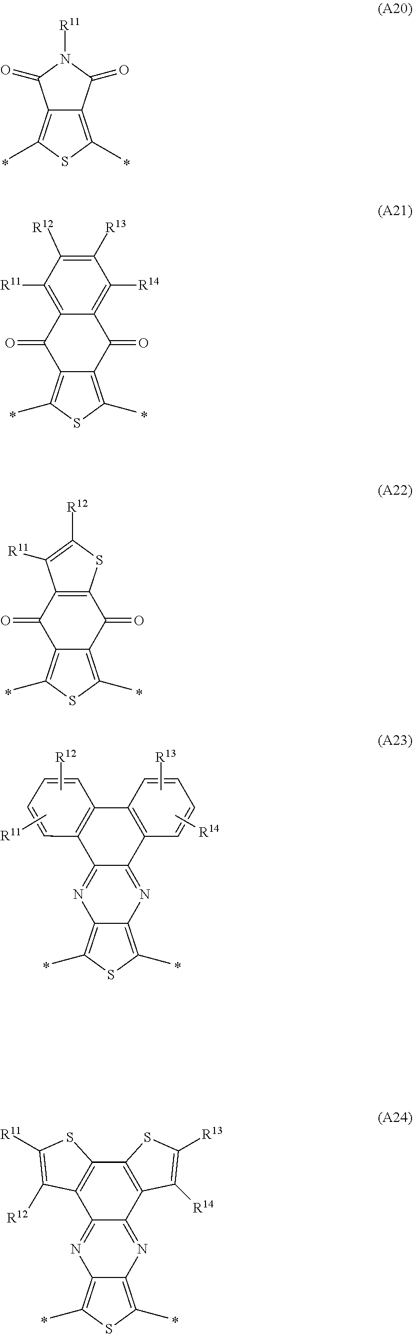

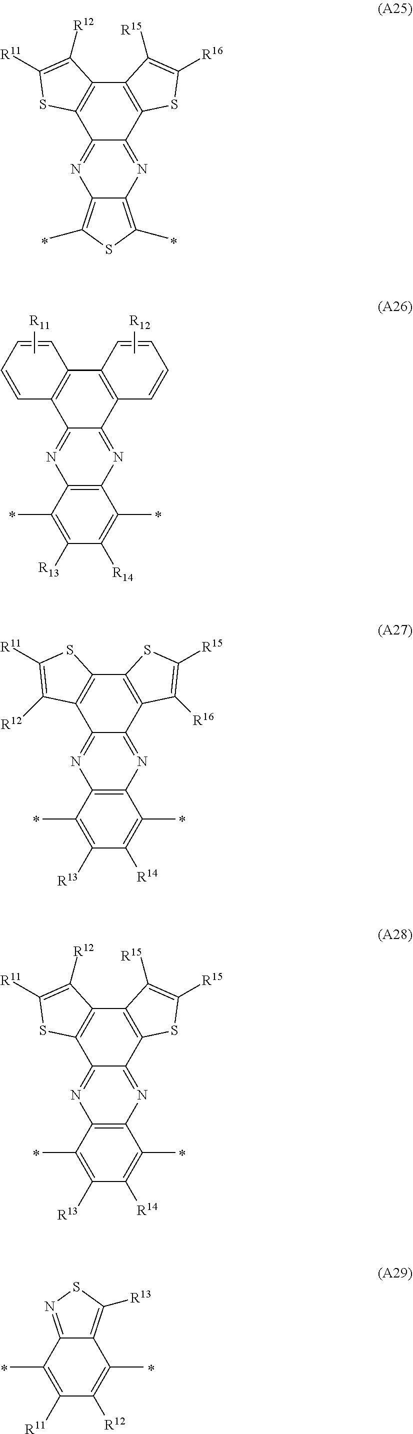

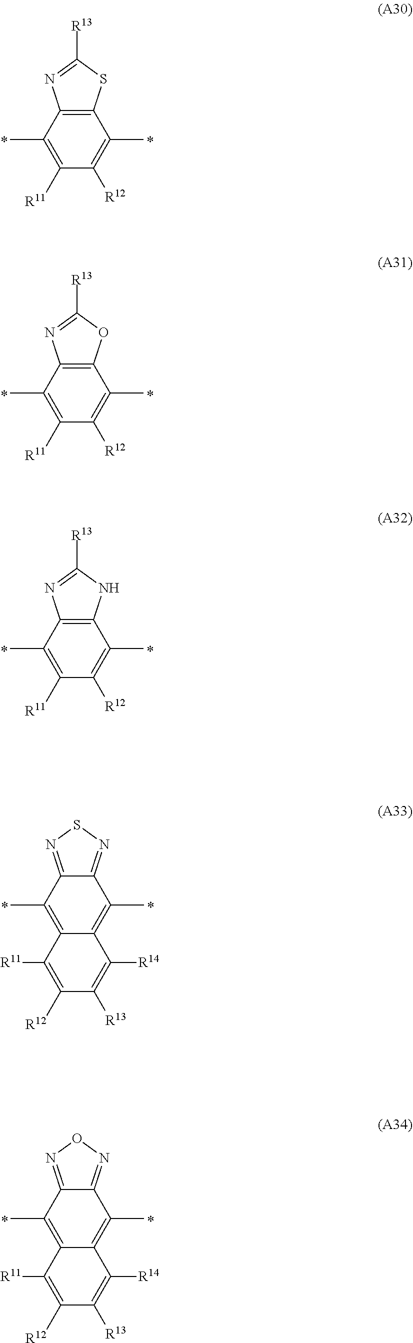

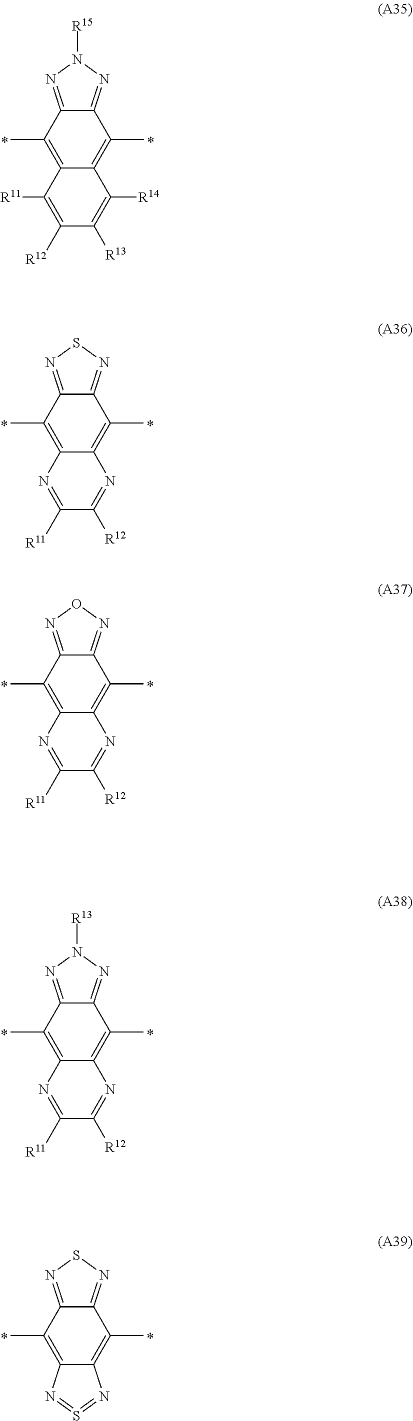

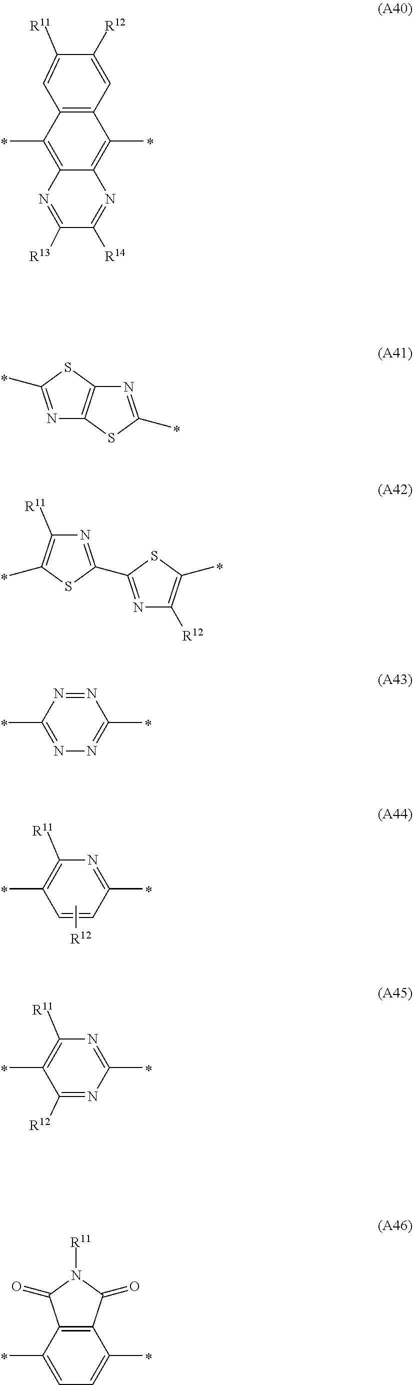

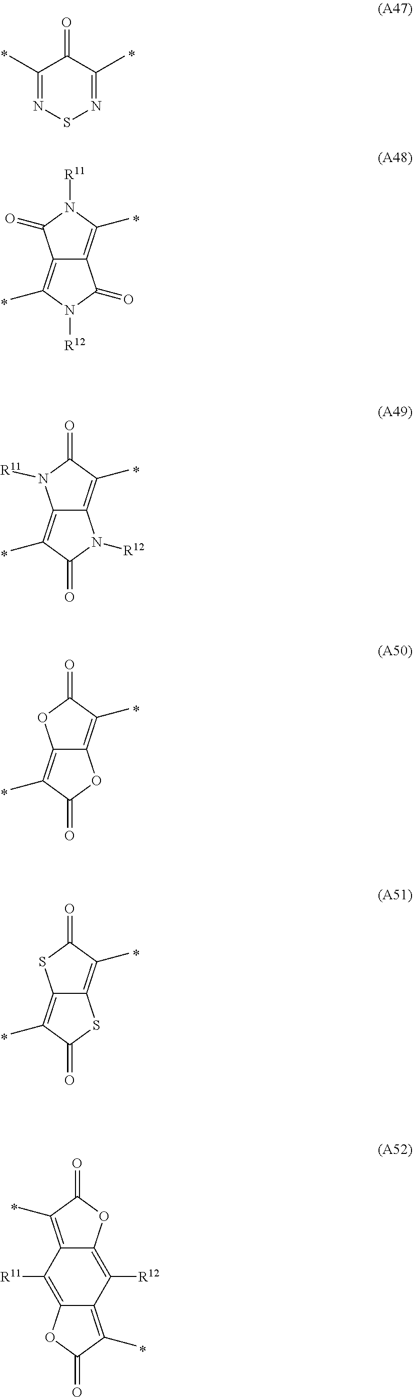

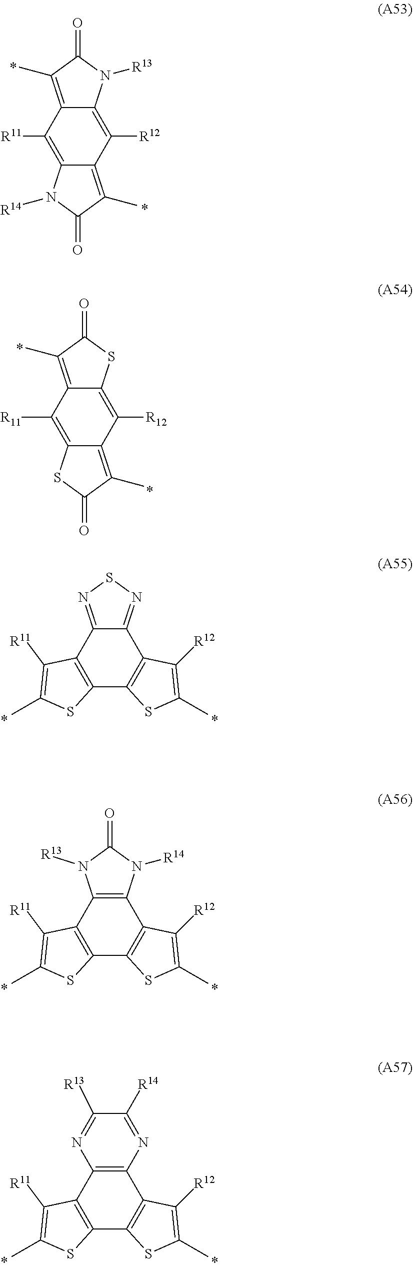

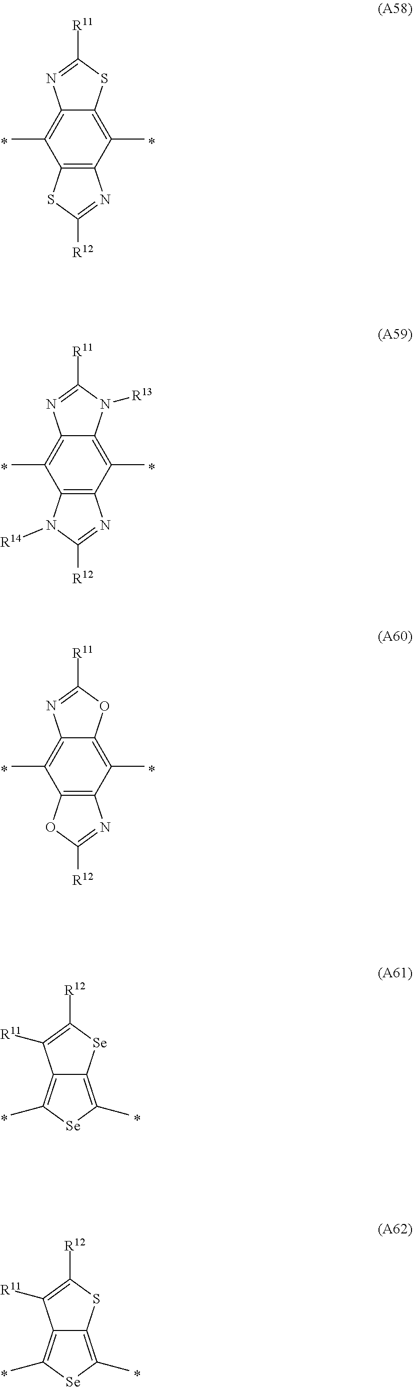

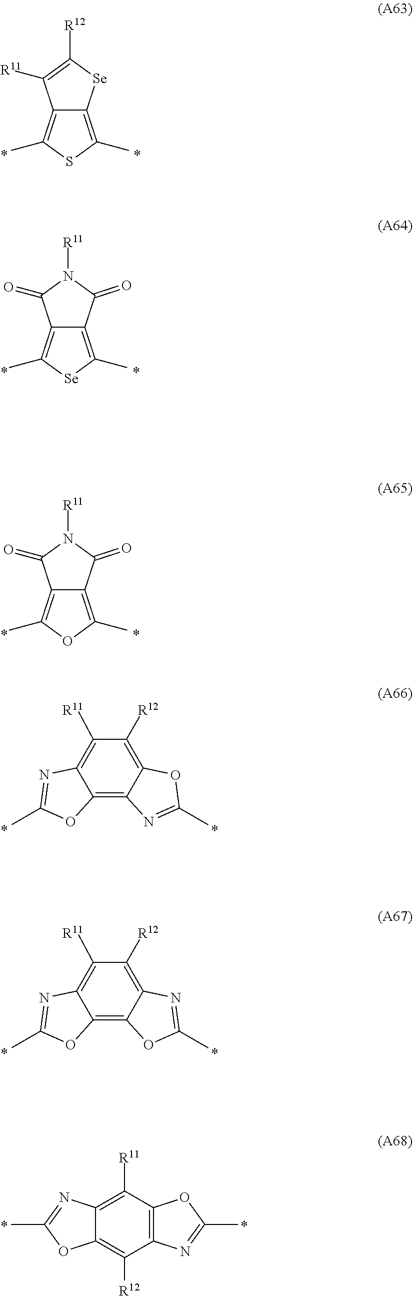

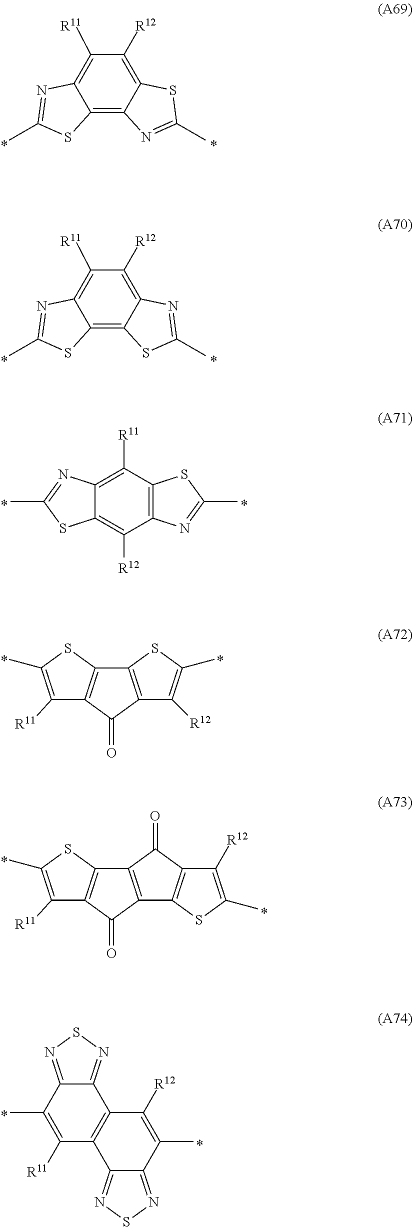

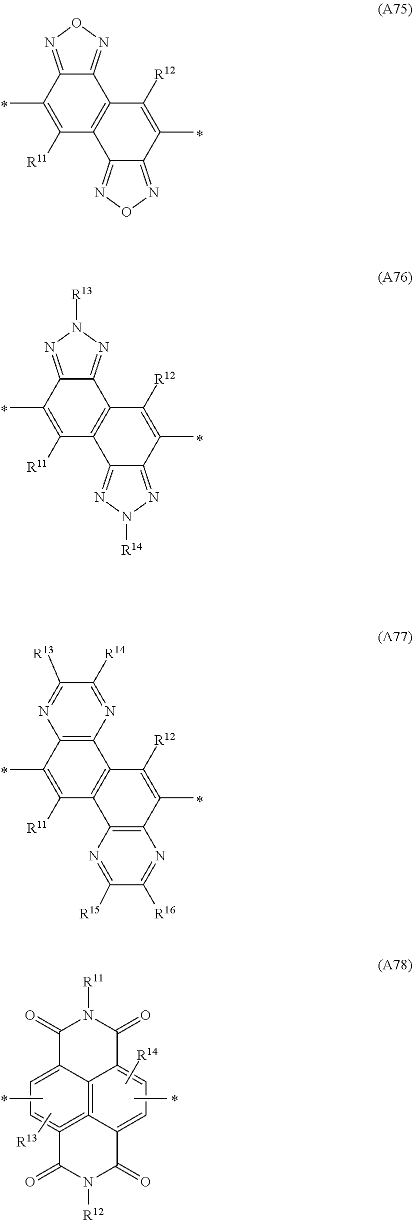

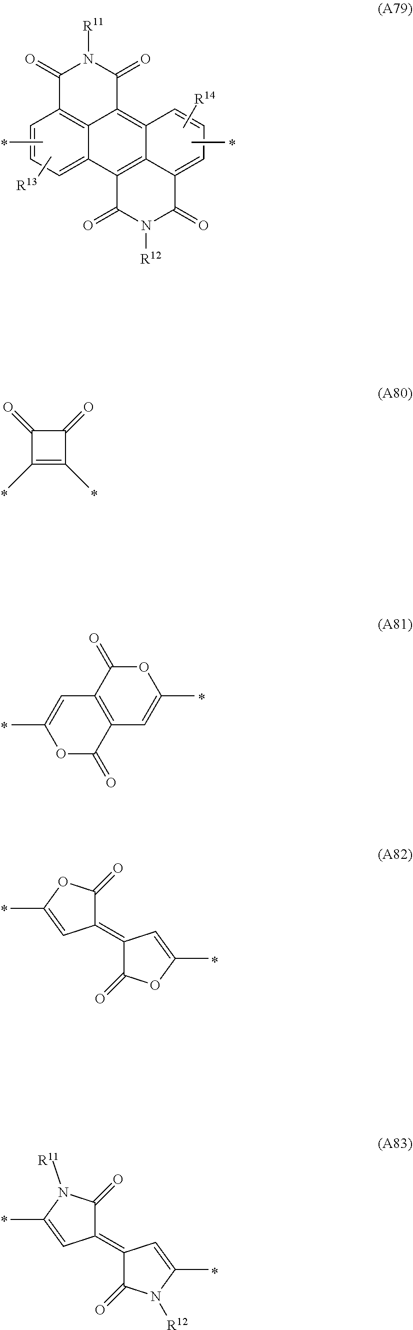

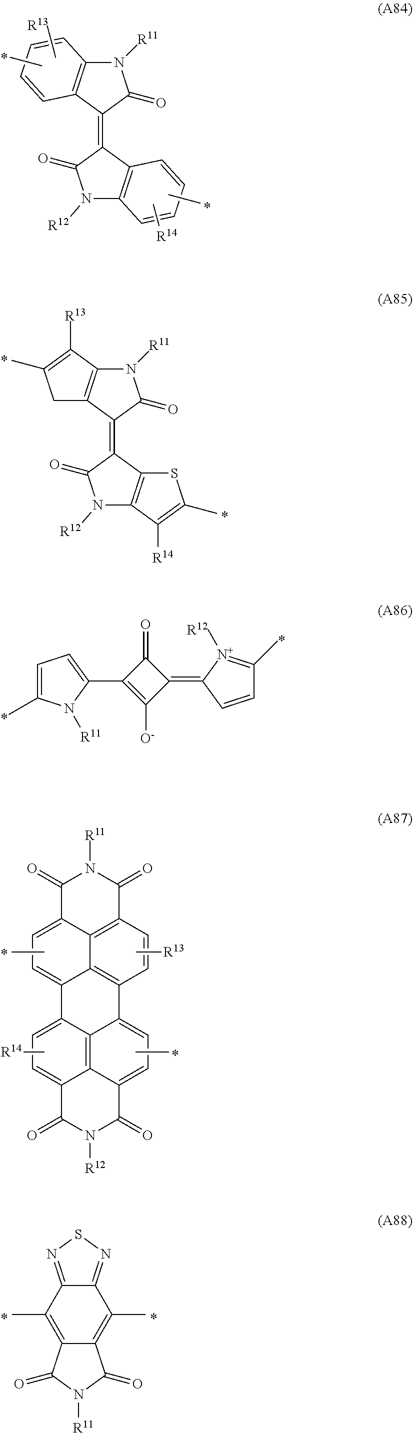

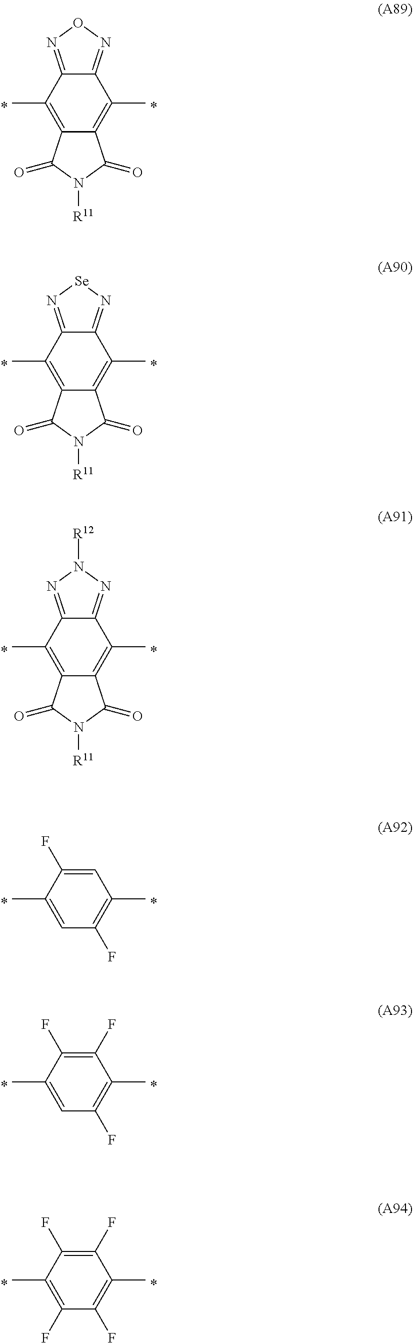

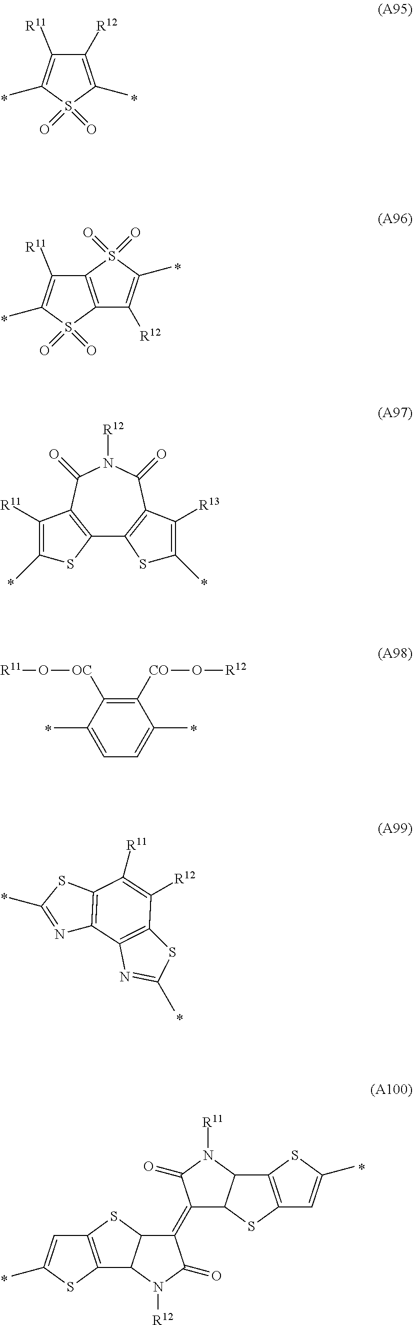

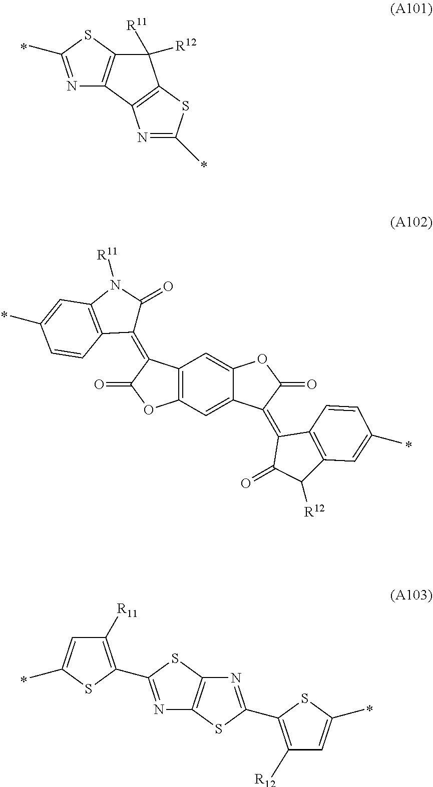

wherein R¹¹, R¹², R¹³, R¹⁴, R¹⁵ and R¹⁶ independently of each other denote H or have one of the meanings of L, R¹ or R² as defined above and below.

Preferred acceptor units are selected from formulae A1, A6, A7, A15, A16, A20, A36, A74, A84, A88, A92, A94, A98 or A103 wherein preferably at least one of R¹¹, R¹², R¹³ and R¹⁴ is different from H.

Further preferred are repeating units and polymers of formulae II1, II2, II3, III, III1-III8, IV and their subformulae wherein one or more of Ar¹, Ar², Ar³ and Ar⁴ denote arylene or heteroarylene selected from the group consisting of the following formulae

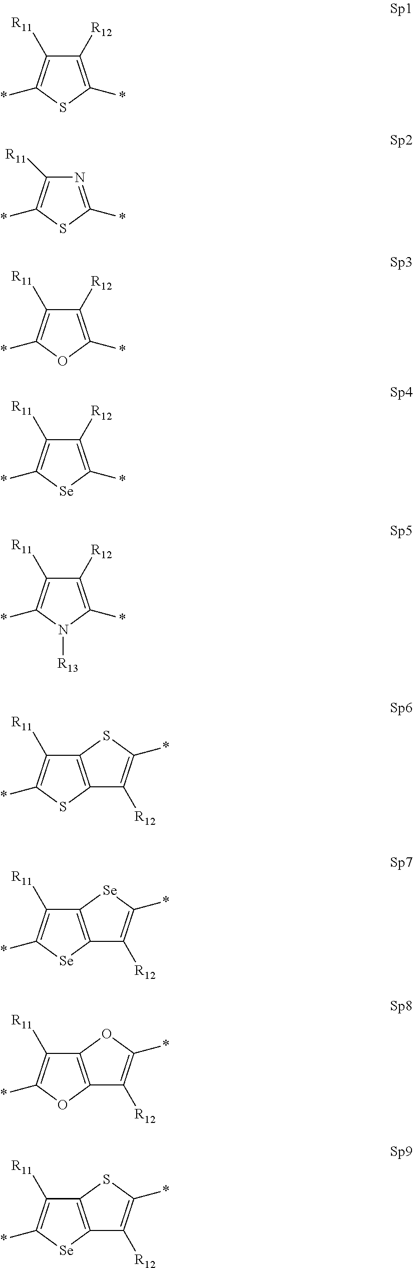

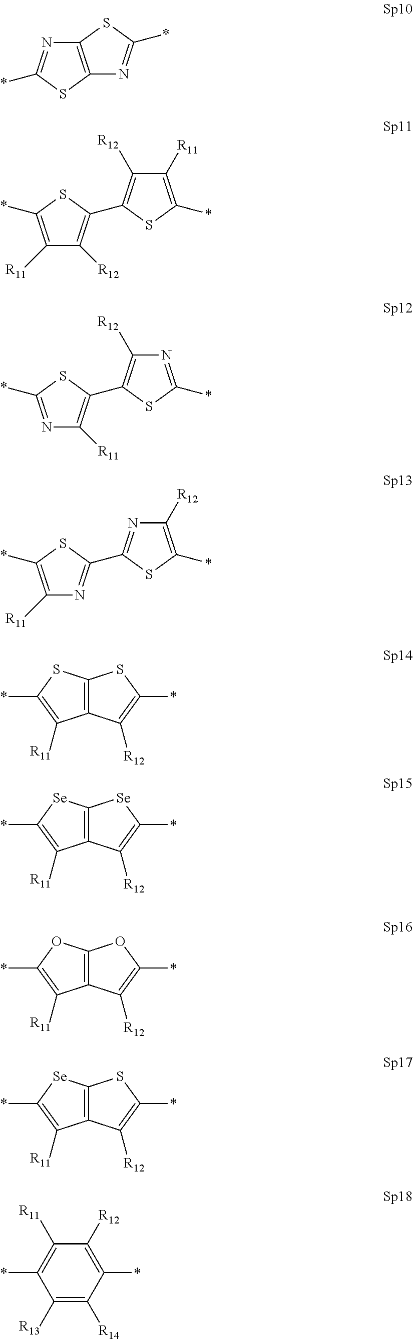

wherein R¹¹, R¹², R¹³, R¹⁴ independently of each other denote H or have one of the meanings of L, R¹ or R² as defined above.

In the formulae Sp1 to Sp17 preferably R¹¹ and R¹² are H. In formula Sp18 preferably R¹¹⁻¹⁴ are H or F.

Very preferred are units selected from formulae Sp1, Sp2, Sp6, Sp10, Sp11, Sp12, Sp13 and Sp14, wherein preferably one of R¹¹ and R¹² is H or both R¹¹ and R¹² are H.

Further preferred are repeating units and polymers of formulae II1, II2, III, III1-III8 and their subformulae wherein
a) one or more of Ar¹, Ar², Ar³ and Ar⁴ denote arylene or heteroarylene, preferably having electron donor properties, selected from the group consisting of the formulae D1-D151, very preferably of the formulae D1, D7, D10, D11, D19, D22, D29, D30, D35, D36, D37, D44, D55, D84, D87, D88, D89, D93, D94, D106, D111, D139, D140, D141, D146 and D150, and/or
b) one or more of Ar¹, Ar², Ar³ and Ar⁴ denote arylene or heteroarylene, preferably having electron acceptor properties, selected from the group consisting of the formulae A1-A103, very preferably of the formulae A1, A6, A7, A15, A16, A20, A36, A74, A84, A88, A92, A94, A98 and A103, and
c) one or more of Ar¹, Ar², Ar³ and Ar⁴ denote arylene or heteroarylene selected from the group consisting of the formulae Sp1-Sp18, very preferably of the formulae Sp1, Sp2, Sp6, Sp10, Sp11, Sp12, Sp13 and Sp14.

Further preferred are polymers of subformulae III1-III8 wherein Ar¹ and Ar² have the same meaning and are selected from formulae D1, D7, D10, D11, D19, D22, D29, D30, D35, D36, D44, D55, D84, D87, D88, D89, D93, D106, D111, D140, D141, D146 and D150.

Further preferred polymers of formula III are selected from the following subformulae

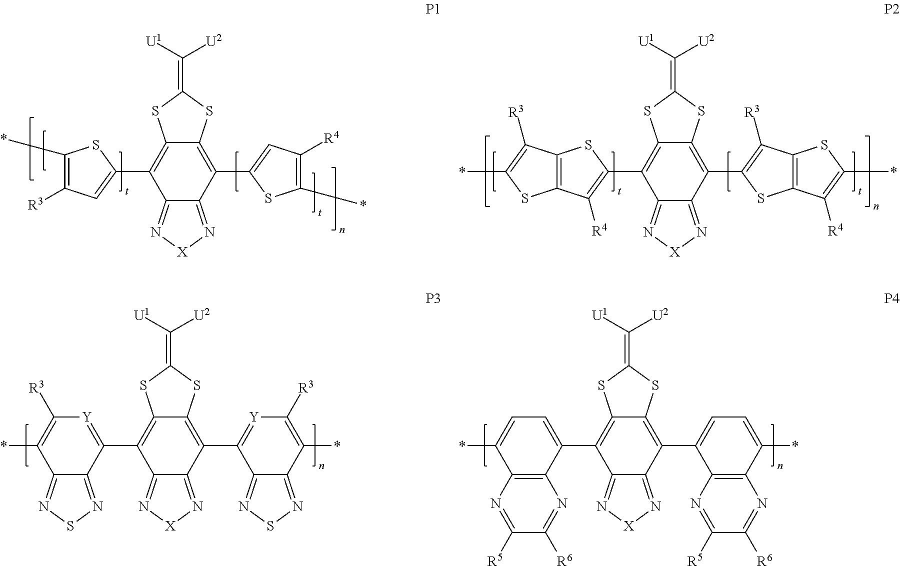

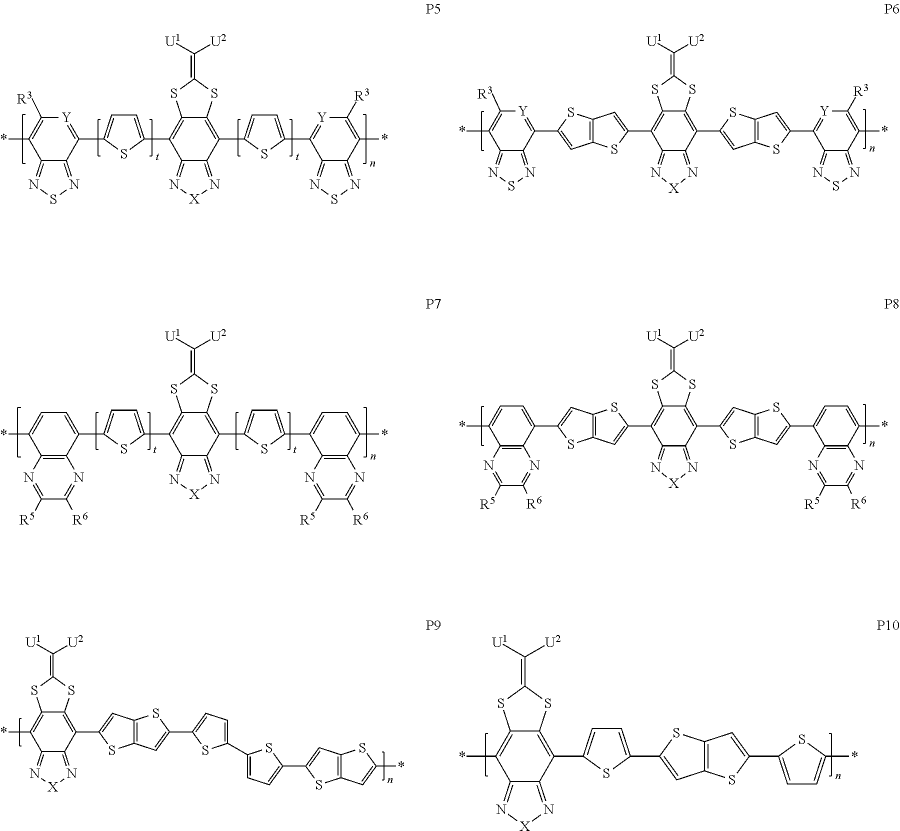

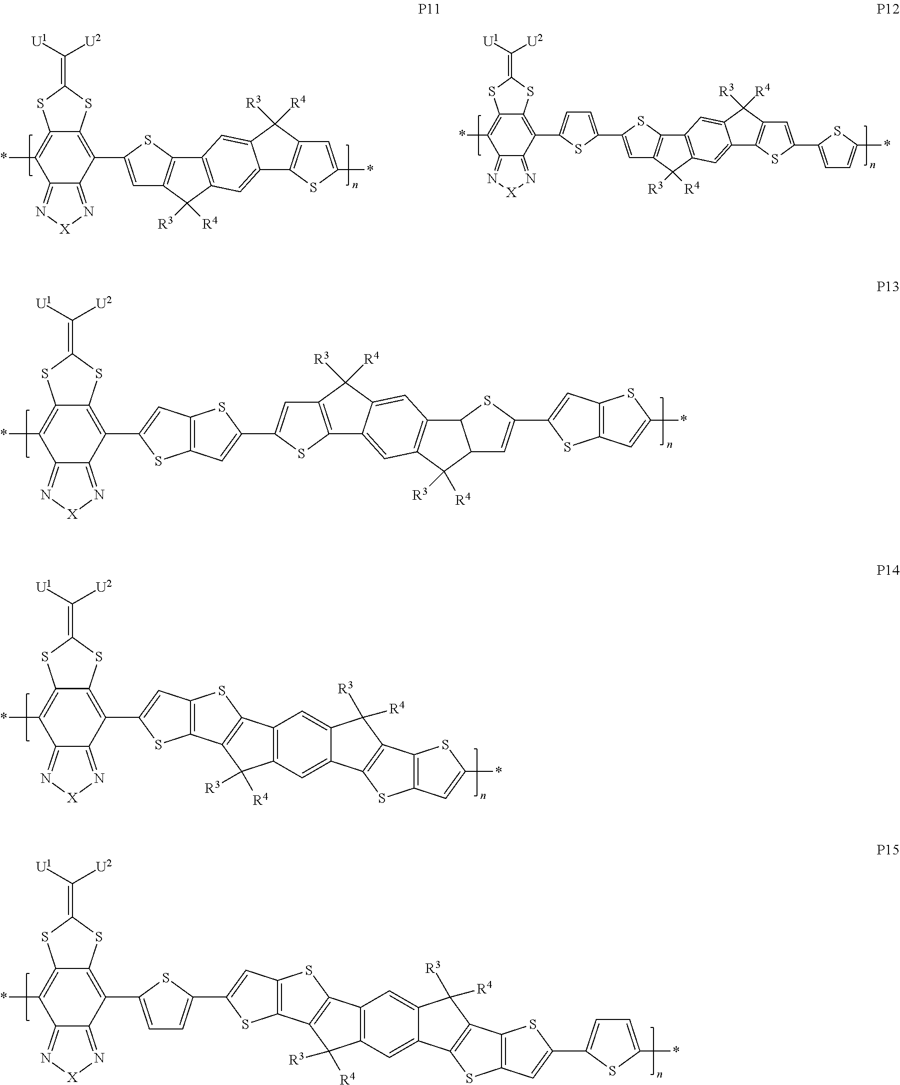

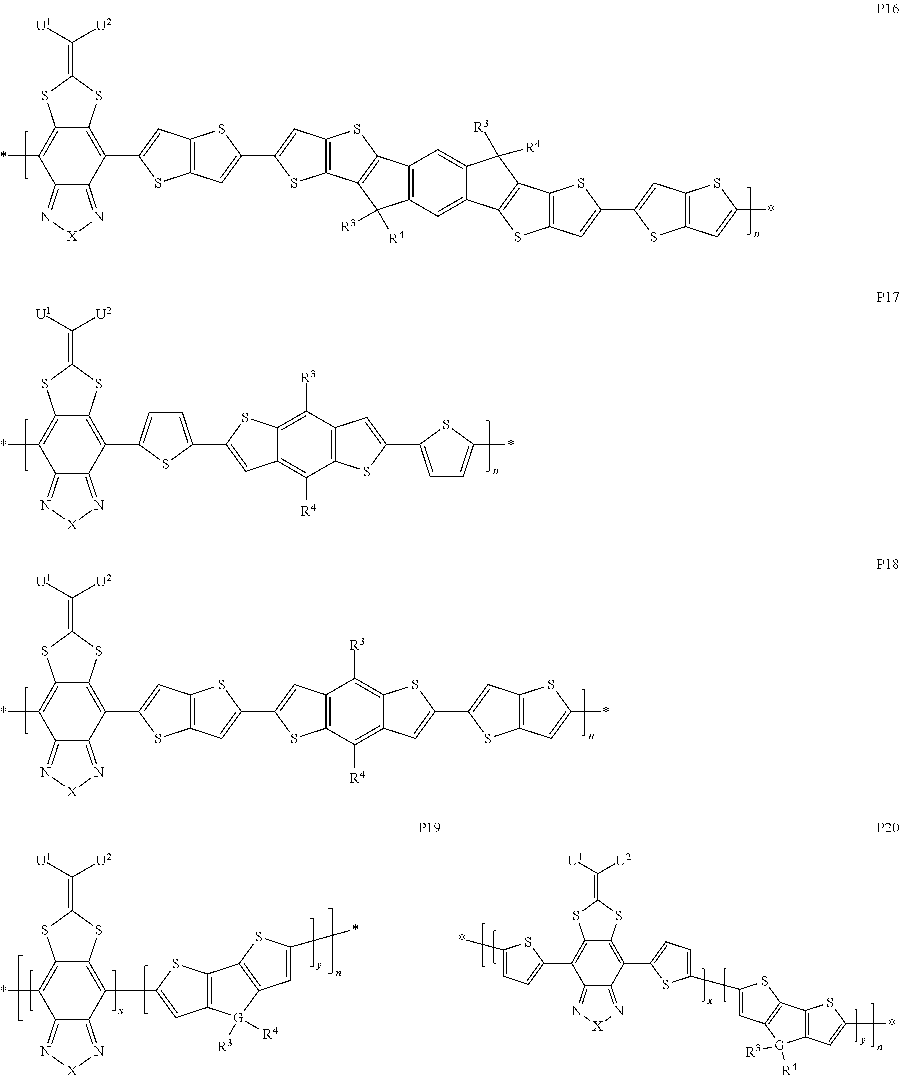

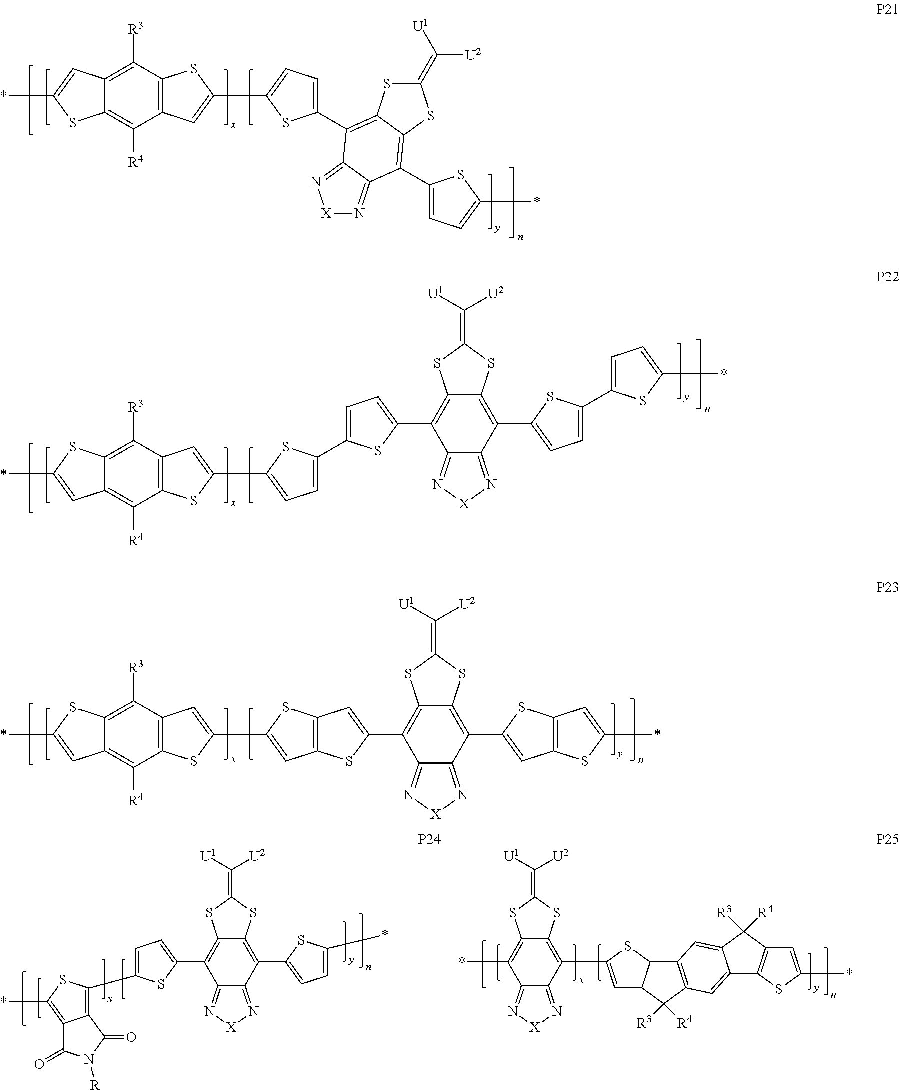

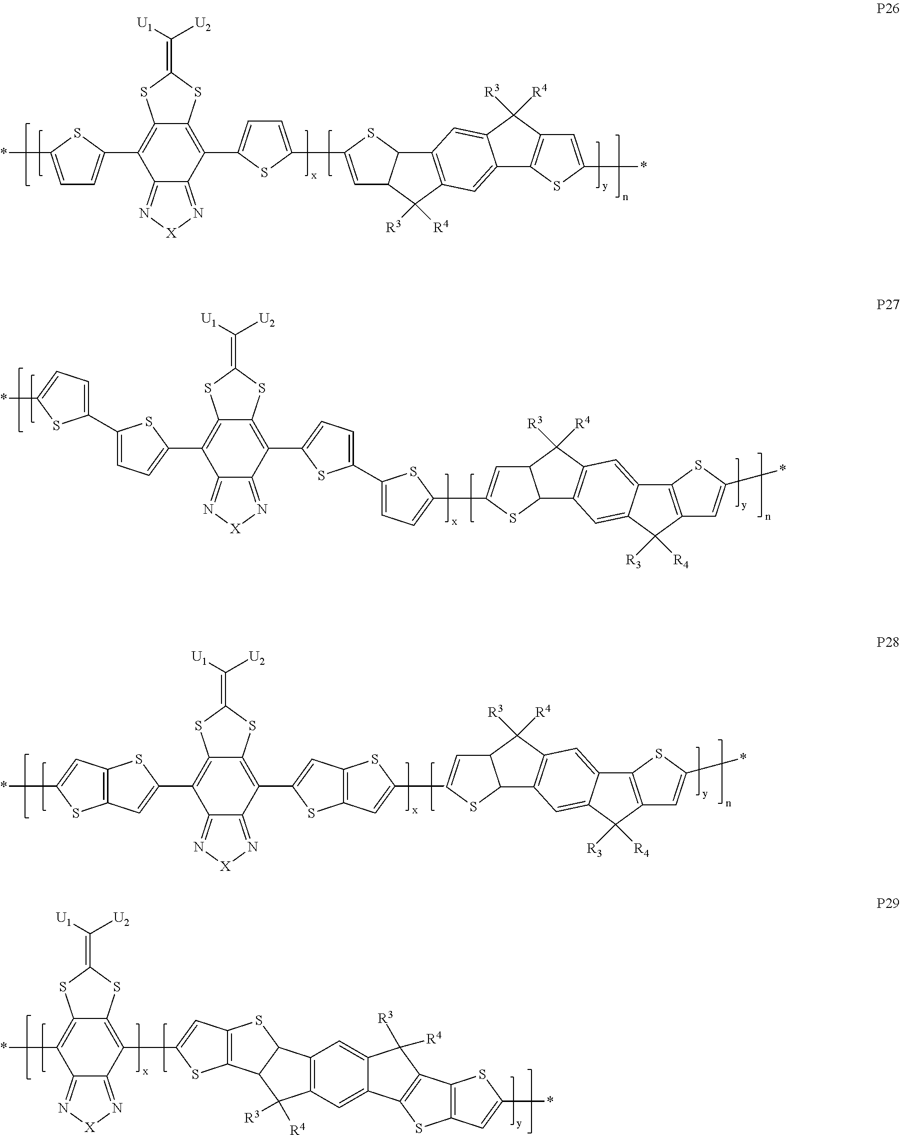

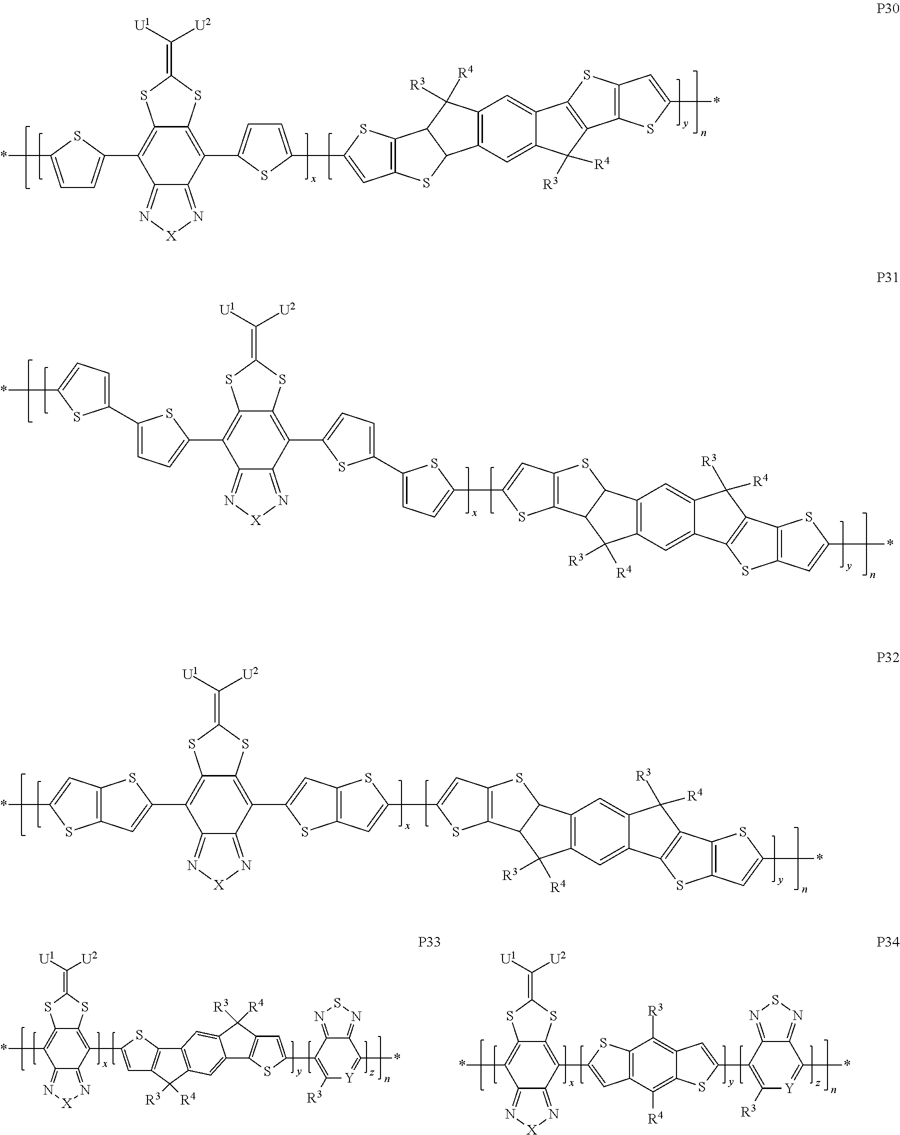

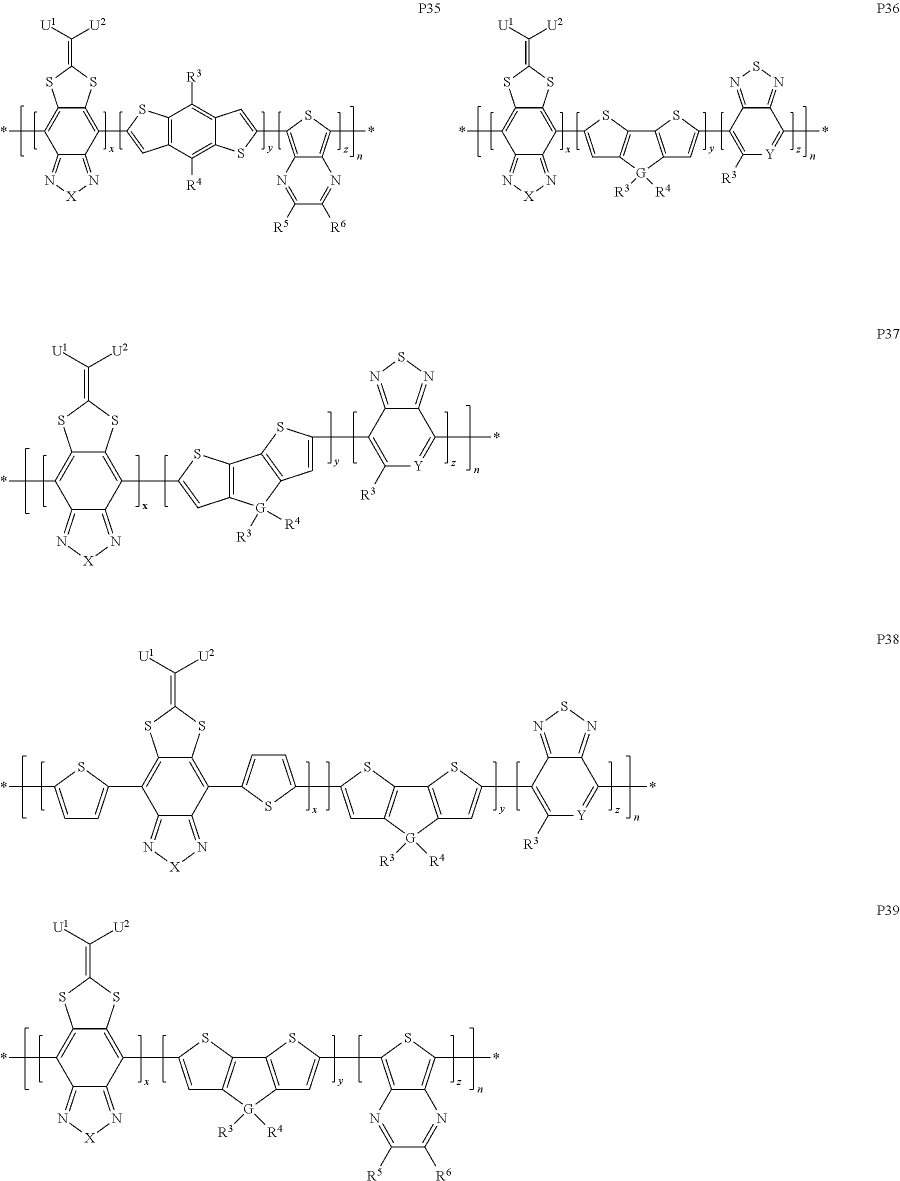

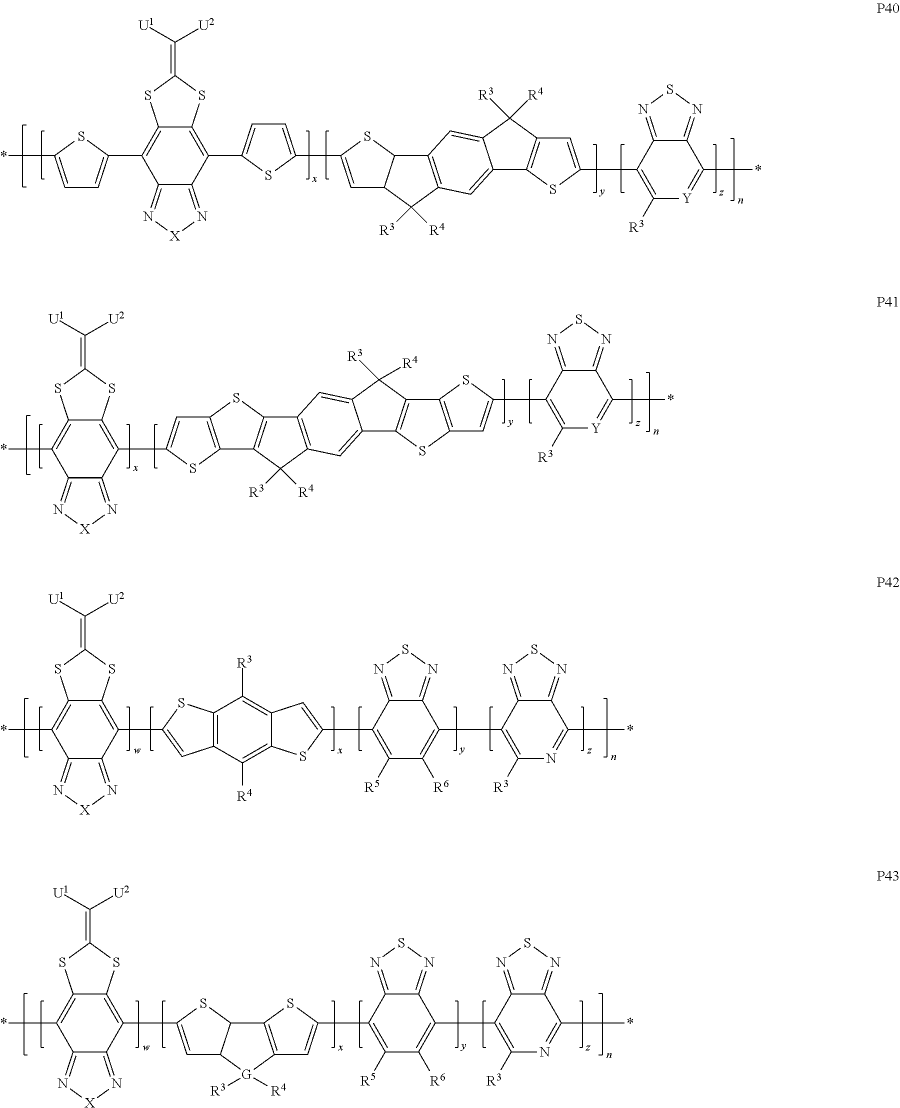

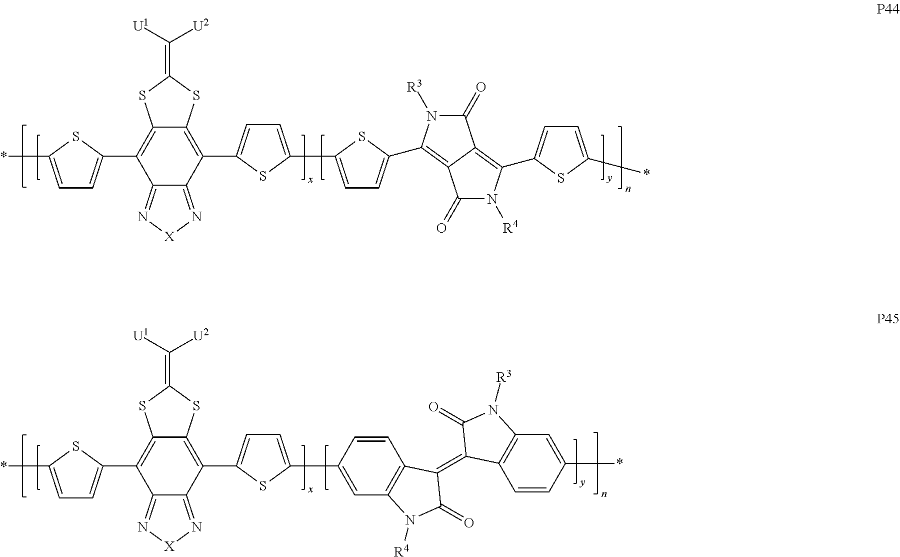

wherein X, U¹, U², w, x, y, z and n are as defined above and below, Y is N or CR⁴, G is C, Si or Ge, t is 1, 2, 3 or 4, preferably 1, 2 or 4, very preferably 1 or 2, R³ and R⁴ have independently of each other and on each occurrence identically or differently one of the meanings given for R¹, and R⁵ and R⁶ have independently of each other and on each occurrence identically or differently one of the meanings given for R¹ and R².

Preferred polymers of formula P1-P45 are selected from the following subformulae

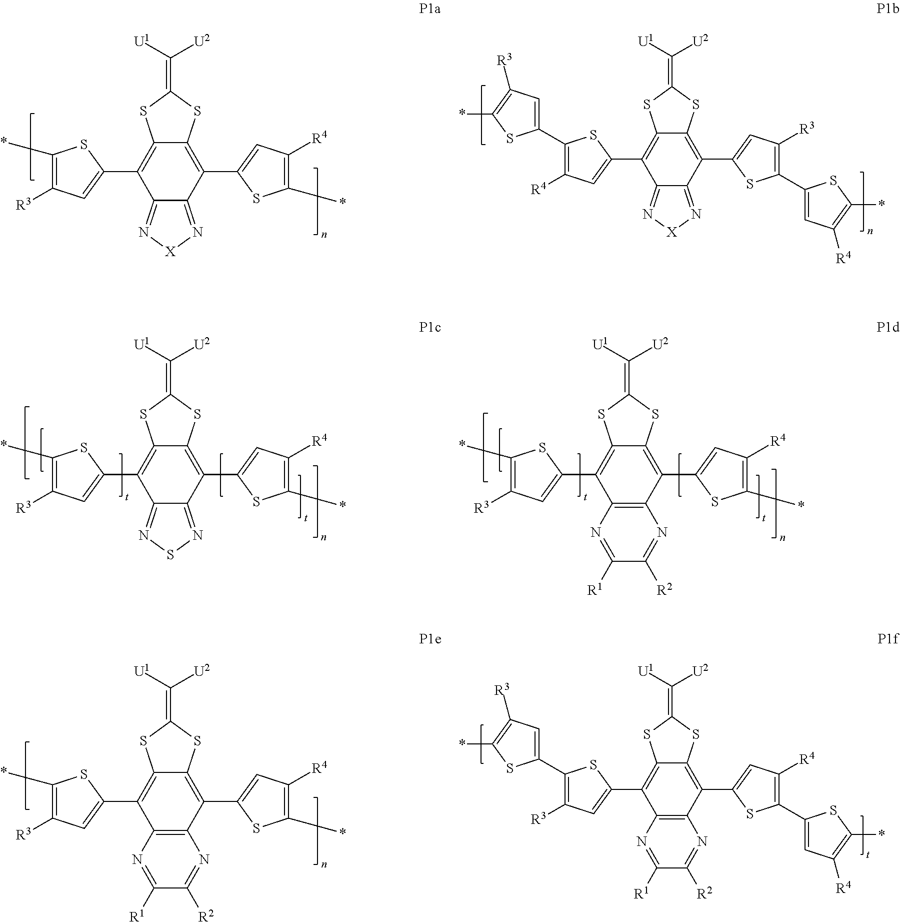

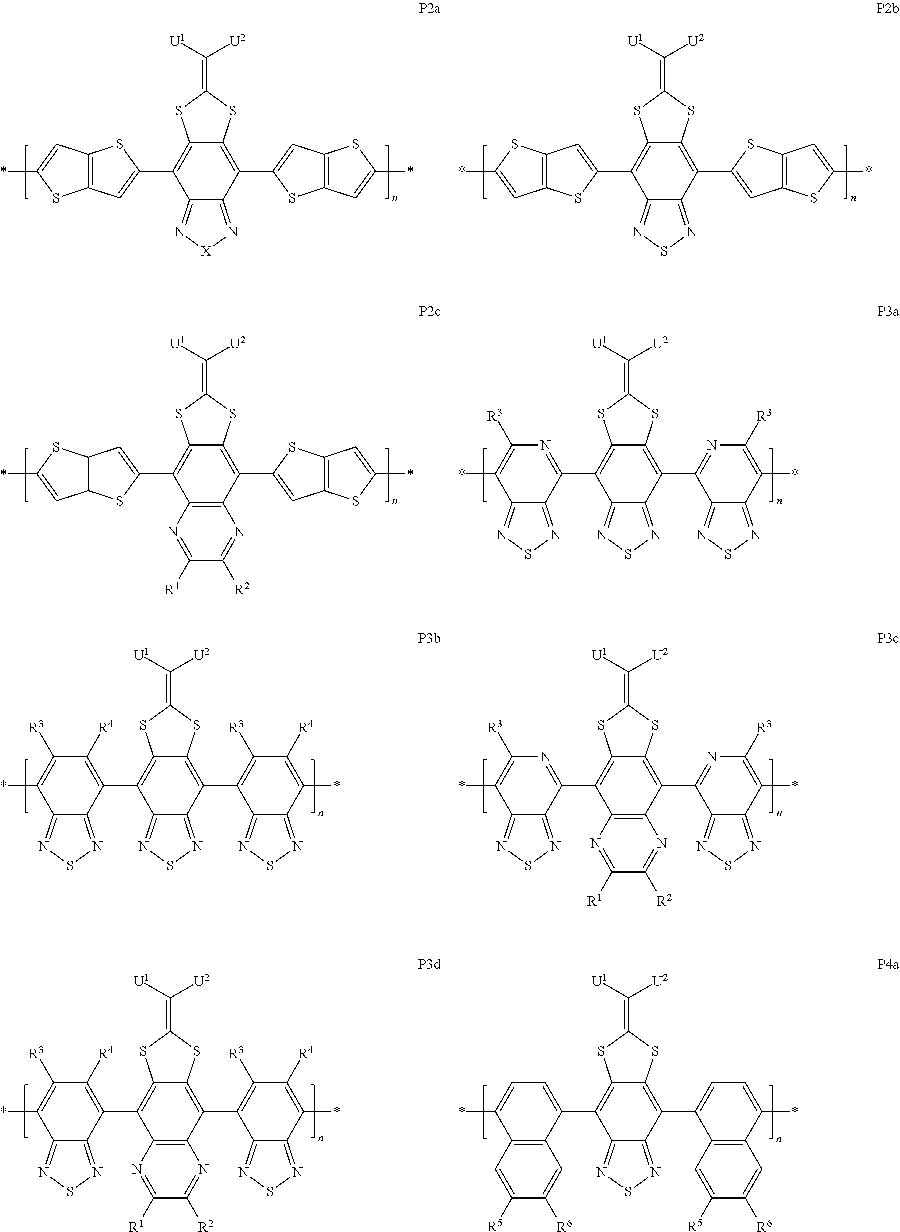

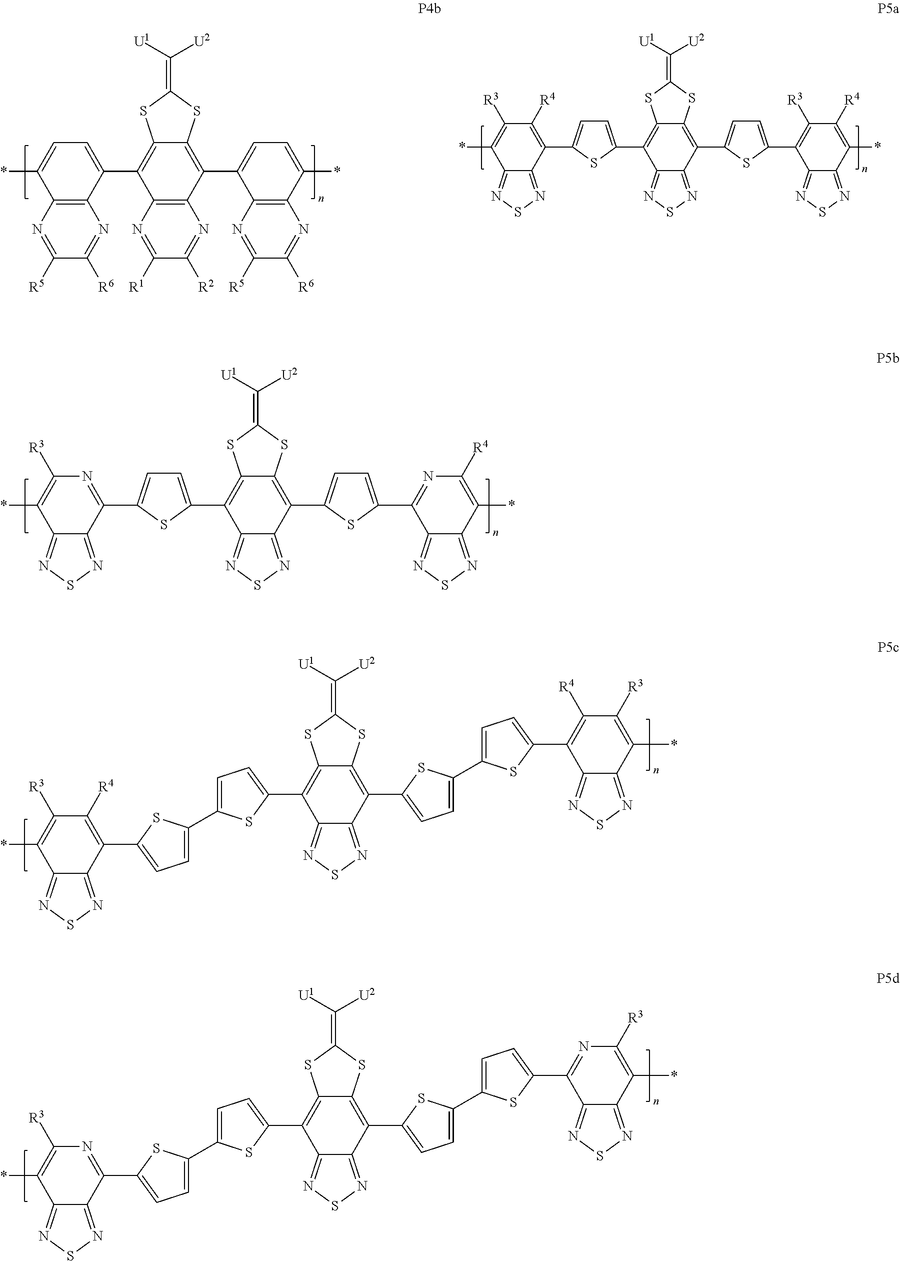

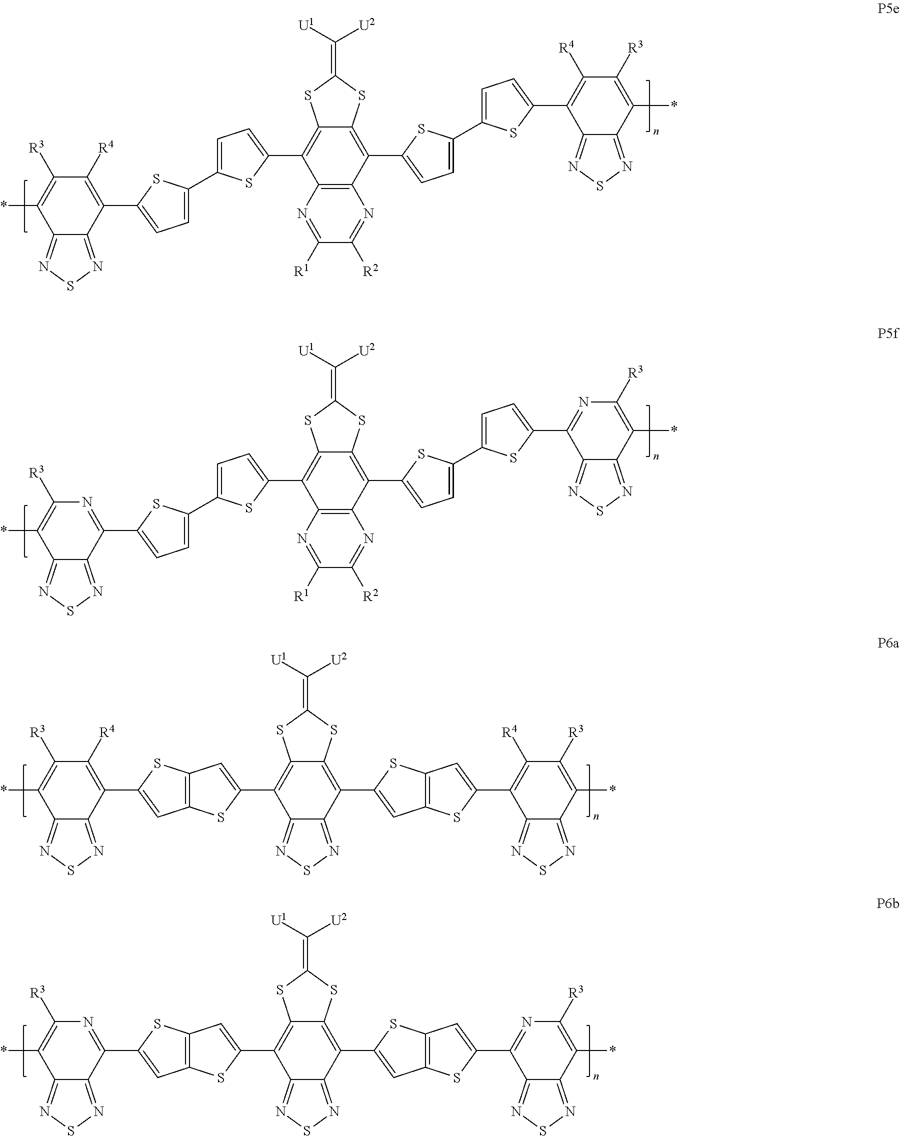

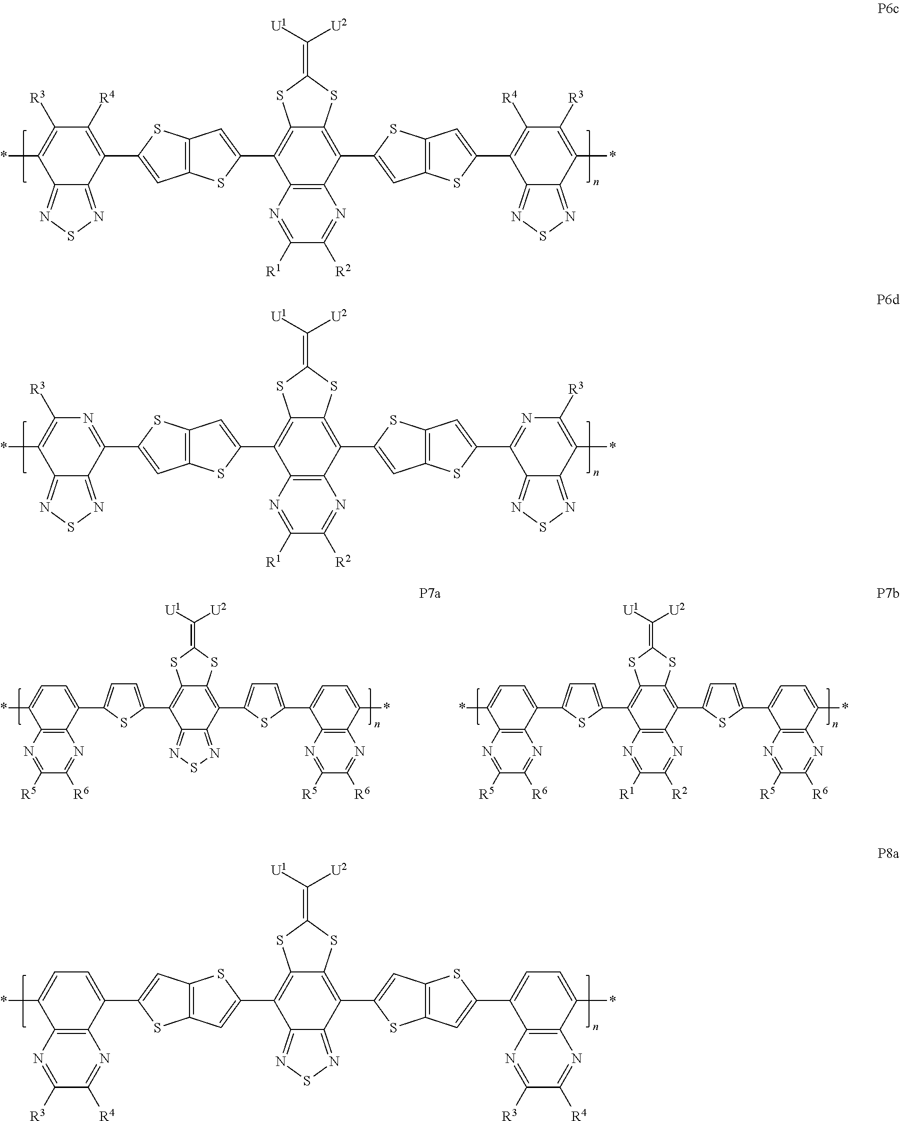

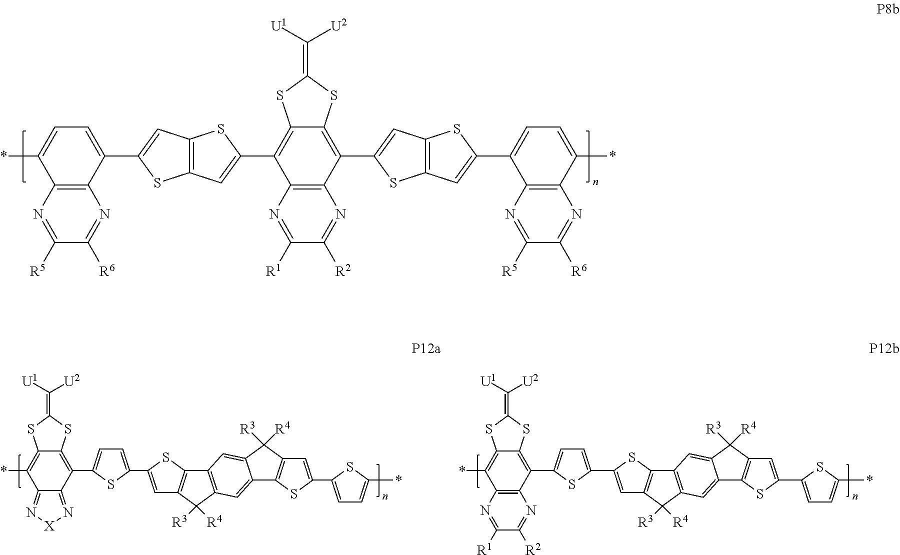

wherein X, U¹, U², R³, R⁴, R⁵, R⁶ and n are as defined for formulae P1-P45.

In formulae P1-P45 X is preferably O or S, NH or CR¹═CR², very preferably S or CR¹═CR²,

In formulae P1-P45 and P1a-P12b CR¹═CR² is preferably CH═CH, and CR⁴ is preferably CH.

In formulae P1-P45 and P1a-P12b U¹ and U² are preferably electron withdrawing groups, very preferably —CN, —COOR or —COR with R being as defined above.

Further preferably the conjugated polymer is selected of formula IV

R²¹-chain-R²² IV

wherein “chain” denotes a polymer chain selected of formulae III, III1-III8, P1-P18 and P1a-P12b, and R²¹ and R²² have independently of each other one of the meanings of L as defined above, or denote, independently of each other, H, F, Br, Cl, 1, —CH₂Cl, —CHO, —CR′═CR″₂, —SiR′R″R′″, —SiR′X′X″, —SiR′R″X′, —SnR′R″R′″, —BR′R″, —B(OR′)(OR″), —B(OH)₂, —O—SO₂—R′, —C≡CH, —C≡C—SiR′₃, —ZnX′ or an endcap group, X′ and X″ denote halogen, R′, R″ and R′″ have independently of each other one of the meanings of R⁰ given in formula I, and preferably denote alkyl with 1 to 12 C atoms, and two of R′, R″ and R′″ may also form a cyclosilyl, cyclostannyl, cycloborane or cycloboronate group with 2 to 20 C atoms together with the respective hetero atom to which they are attached.

Preferred endcap groups R²¹ and R²² are H, C₁₋₂₀ alkyl, or optionally substituted C₆₋₁₂ aryl or C₂₋₁₀ heteroaryl, very preferably H or phenyl.

Another preferred embodiment of the present invention relates to a small molecule or oligomer of formula VI

R^{T1}—(Ar¹)ₑ—(Ar²)_{f}—[(Ar³)_{g}—(Ar⁴)ₕ—U—(Ar⁵)ᵢ—(Ar⁶)^{k}]ₒ—(Ar⁷)ₗ—(Ar⁸)ₘ—R^{T2} VI

wherein the individual radicals, independently of each other and on each occurrence identically or differently, have the following meanings
U a unit of formula I or its subformulae,
Ar¹⁻⁸ arylene or heteroarylene that has from 5 to 20 ring atoms, is mono- or polycyclic, optionally contains fused rings, and is unsubstituted or substituted by one or more identical or different groups L or R¹ as defined in formula I, or —CY¹═CY²— or —C≡C—,
Y¹, Y² H, F, Cl or CN,
R^{T1}, R^{T2} a carbyl or hydrocarbyl group with 1 to 30 C atoms that is optionally substituted by one or more groups L and optionally comprises one or more hetero atoms,
e-m 0 or 1, preferably with at least one of e-m being 1,
o 1, 2 or 3.

Preferred groups R^{T1} and R^{T2} in formula I are selected from H, F, Cl, Br, —NO₂, —CN, —CF₃, R*, —CF₂—R*, —O—R*, —S—R*, —SO₂—R*, —SO₃—R*, —C(═O)—H, —C(═O)—R*, —C(═S)—R*, —C(═O)—CF₂—R*, —C(═O)—OR*, —C(═S)—OR*, —O—C(═O)—R*, —O—C(═S)—R*, —C(═O)—SR*, —S—C(═O)—R*, —C(═O)NR*R**, —NR*—C(═O)—R*, —NHR*, —NR*R**, —CR*═CR*R**, —C≡C—R*, —C≡C—SiR*R**R***, —SiR*R**R***, —CH═CH(CN), —CH═C(CN)₂, —C(CN)═C(CN)₂, —CH═C(CN)(R^{a}), CH═C(CN)—C(═O)—OR*, —CH═C(CO—OR*)₂, —CH═C(CO—NR*R**)₂, and the group consisting of the following formulae

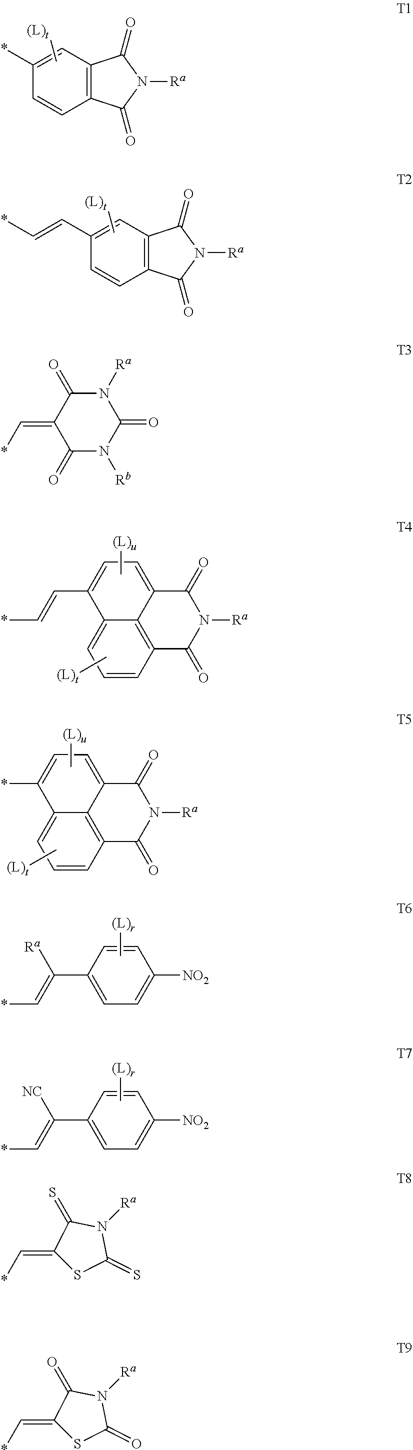

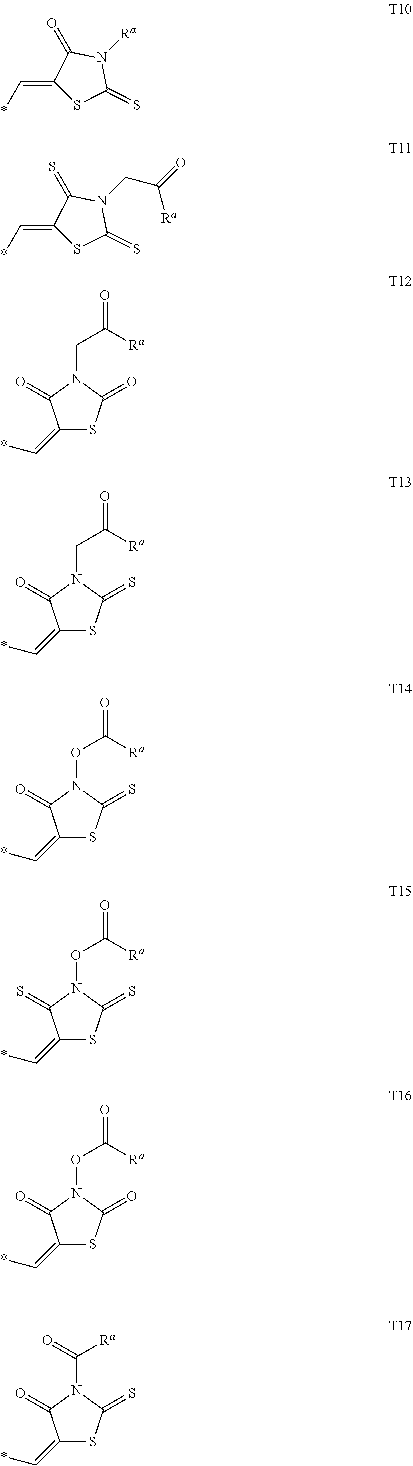

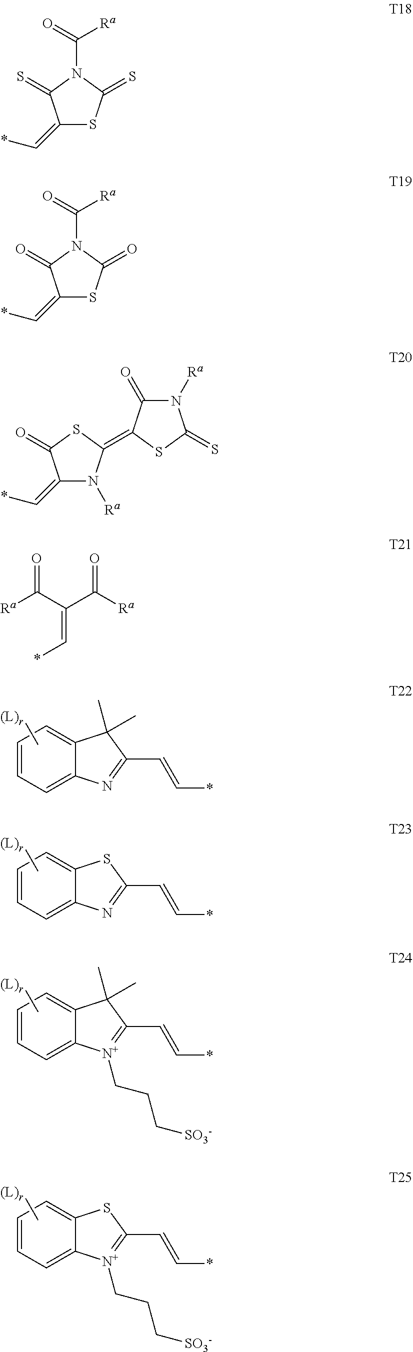

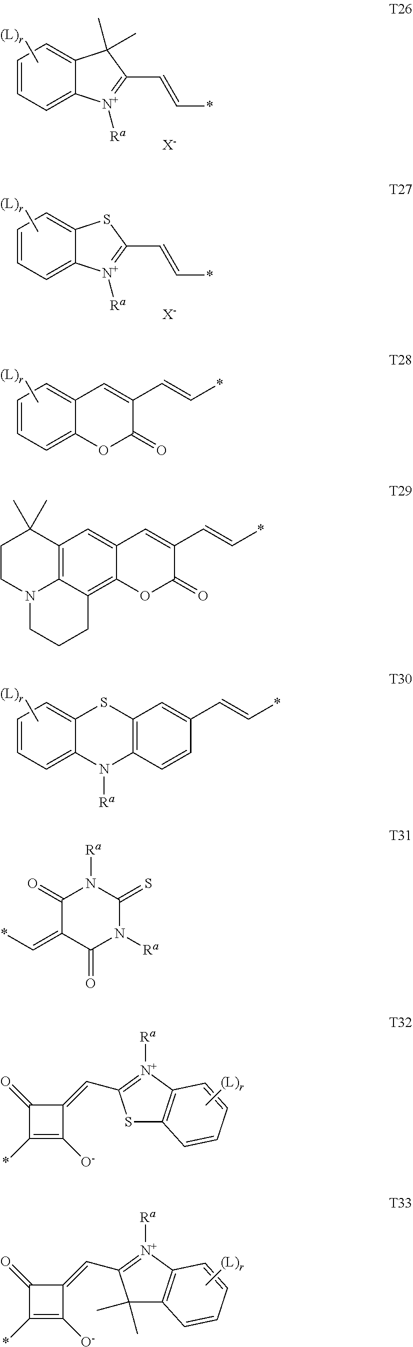

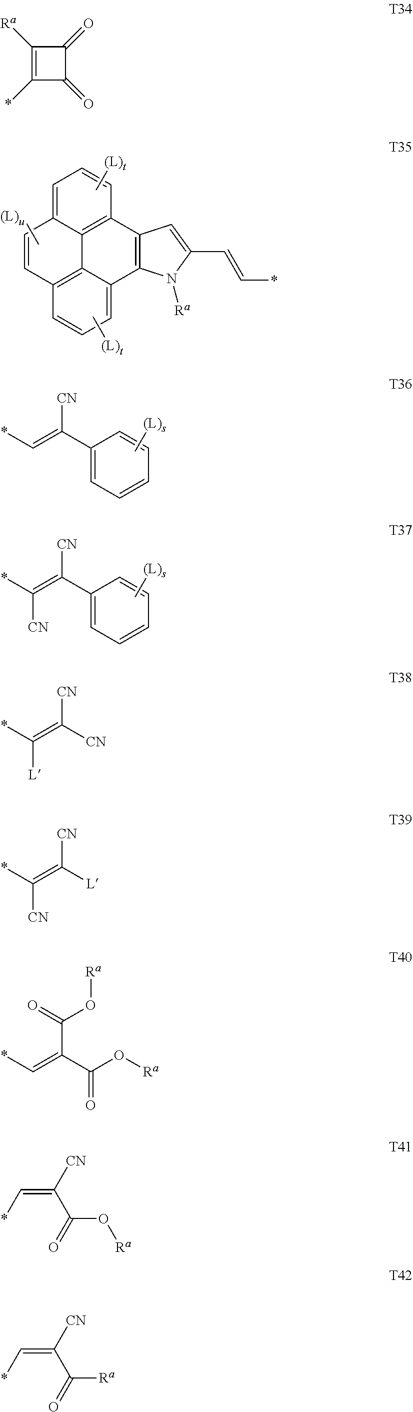

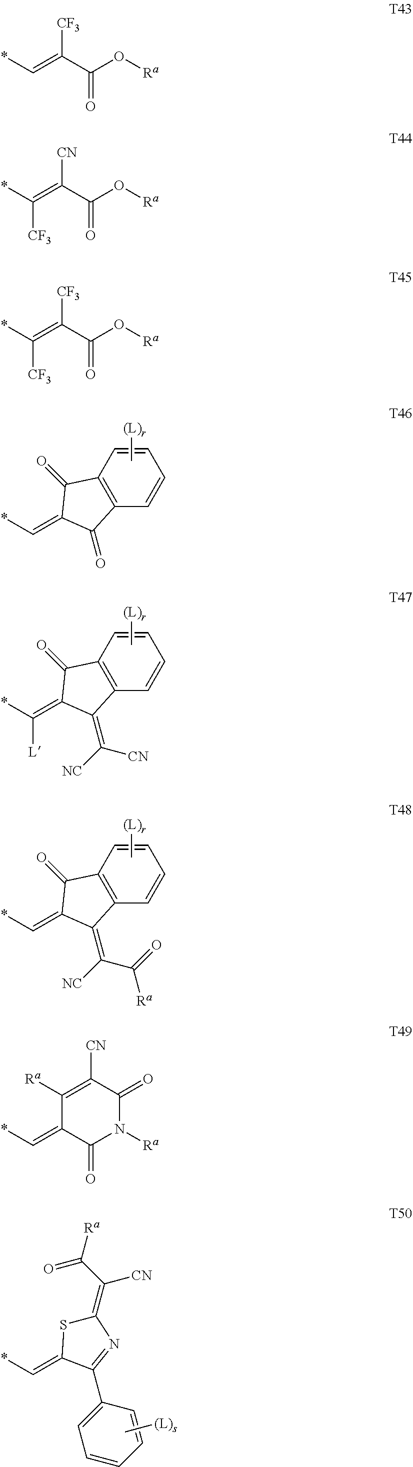

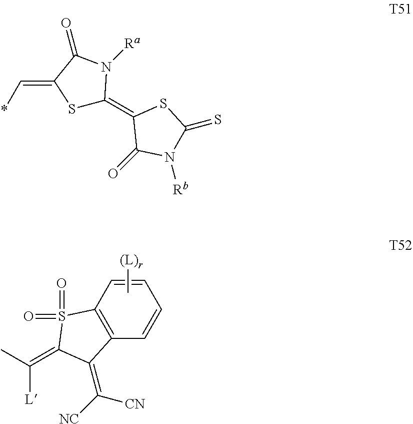

wherein the individual radicals, independently of each other and on each occurrence identically or differently, have the following meanings
R^{a}, R^{b} aryl or heteroaryl, each having from 4 to 30 ring atoms, optionally containing fused rings and being unsubstituted or substituted with one or more groups L, or one of the meanings given for L,
R*, R**, R*** alkyl with 1 to 20 C atoms which is straight-chain, branched or cyclic, and is unsubstituted, or substituted with one or more F or Cl atoms or CN groups, or perfluorinated, and in which one or more C atoms are optionally replaced by —O—, —S—, —C(═O)—, —C(═S)—, —SiR⁰R⁰⁰—, —NR⁰R⁰⁰—, —CHR⁰═CR⁰⁰— or —C≡C— such that O- and/or S-atoms are not directly linked to each other,
L F, Cl, —NO₂, —CN, —NC, —NCO, —NCS, —OCN, —SCN, R⁰, OR⁰, SR⁰, —C(═O)X⁰, —C(═O)R⁰, —C(═O)—OR⁰, —O—C(═O)—R⁰, —NH₂, —NHR⁰, —NR⁰R⁰⁰, —C(═O)NHR⁰, —C(═O)NR⁰R⁰⁰, —SO₃R⁰, —SO₂R⁰, —OH, —NO₂, —CF₃, —SF₅, or optionally substituted silyl, or carbyl or hydrocarbyl with 1 to 30, preferably 1 to 20, C atoms that is optionally substituted and optionally comprises one or more hetero atoms, preferably F, —CN, R⁰, —OR⁰, —SR⁰, —C(═O)—R⁰, —C(═O)—OR⁰, —O—C(═O)—R⁰, —O—C(═O)—OR⁰, —C(═O)—NHR⁰, —C(═O)—NR⁰R⁰⁰,
L′ H or one of the meanings of L,
R⁰, R⁰⁰ H or straight-chain or branched alkyl with 1 to 20, preferably 1 to 12 C atoms that is optionally fluorinated,
Y¹, Y² H, F, Cl or CN,
X⁰ halogen, preferably F or Cl,
r 0, 1, 2, 3 or 4,
s 0, 1, 2, 3, 4 or 5,
t 0, 1, 2 or 3,
u 0, 1 or 2.

Preferred compounds of formula VI are those wherein one or both, preferably both, of R^{T1} and R^{T2} denote an electron withdrawing group.

Preferred electron withdrawing groups R^{T1} and R^{T2} are selected from —CN, —C(═O)—OR*, —C(═S)—OR*, —CH═CH(CN), —CH═C(CN)₂, —C(CN)═C(CN)₂, —CH═C(CN)(R^{a}), CH═C(CN)—C(═O)—OR*, —CH═C(CO—OR*)₂, and formulae T1-T51.

Very preferred groups R^{T1} and R^{T2} are selected from the following formulae

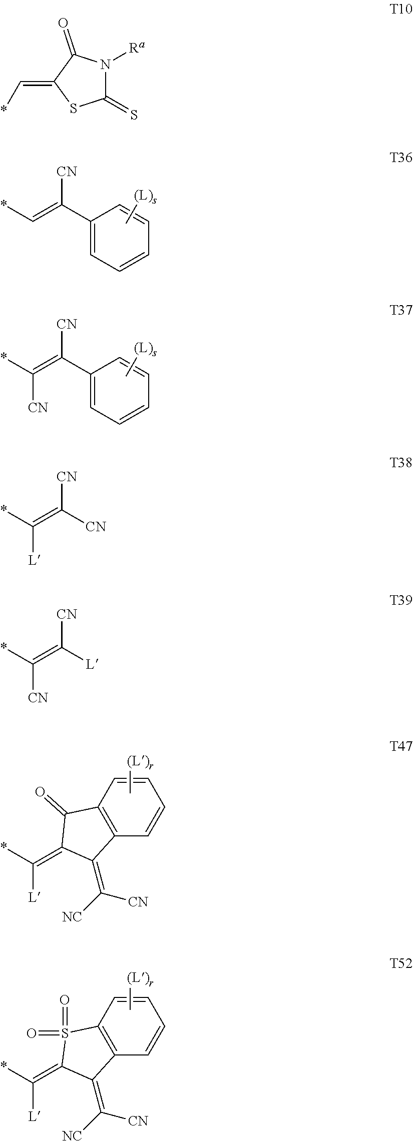

wherein L, L′, R^{a}, r and s have the meanings given above and below. In these formulae preferably L′ is H. Further preferably in these formulae r is 0.

The above formulae T1-T51 are meant to also include their respective E- or Z-stereoisomer with respect to the C═C bond in α-position to the adjacent group Ar^{1.8}, thus for example the group

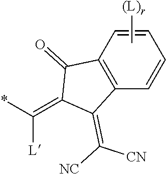

may also denote

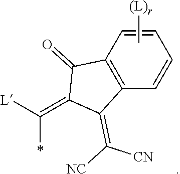

Preferred compounds of formula VI are those wherein Ar¹⁻⁸ are selected from the following groups
a) the group consisting of the formulae D1-D151, very preferably of the formulae D1, D7, D10, D11, D19, D22, D29, D30, D35, D36, D37, D44, D55, D84, D87, D88, D89, D93, D94, D106, D111, D139, D140, D141, D146 and D150,
b) the group consisting of the formulae A1-A103, very preferably of the formulae A1, A6, A7, A15, A16, A20, A36, A74, A84, A88, A92, A94, A98 and A103,
c) the group consisting of the formulae Sp1-Sp18, very preferably of the formulae Sp1, Sp2, Sp6, Sp10, Sp11, Sp12, Sp13 and Sp14.

Further preferred compounds of formula VI are selected from the following preferred embodiments, including any combination thereof:
o is 1,
e, f, l, m are 0, 1 or 2, and g, h, l, k are 0,
Ar¹⁻⁸ are selected from formulae Sp1, Sp2, Sp6, Sp10, Sp11, Sp12, Sp13 and Sp14,
R^{T1} and R^{T2} are selected from formulae T10, T36, T37, T38, T39 and T47,
R^{T1} and R^{T2} are selected from formula T47.

Further preferred are small molecules of formula VI selected from the following subformulae

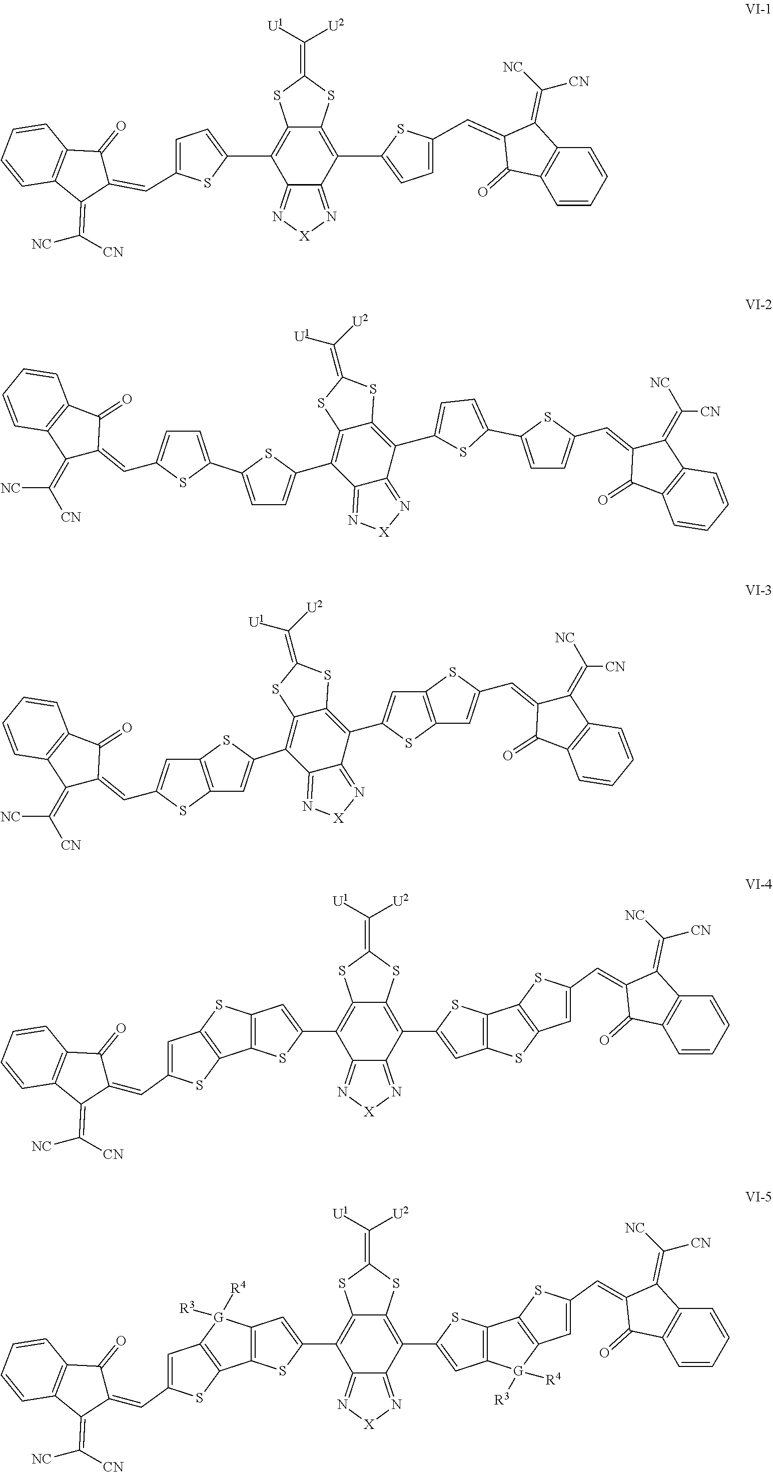

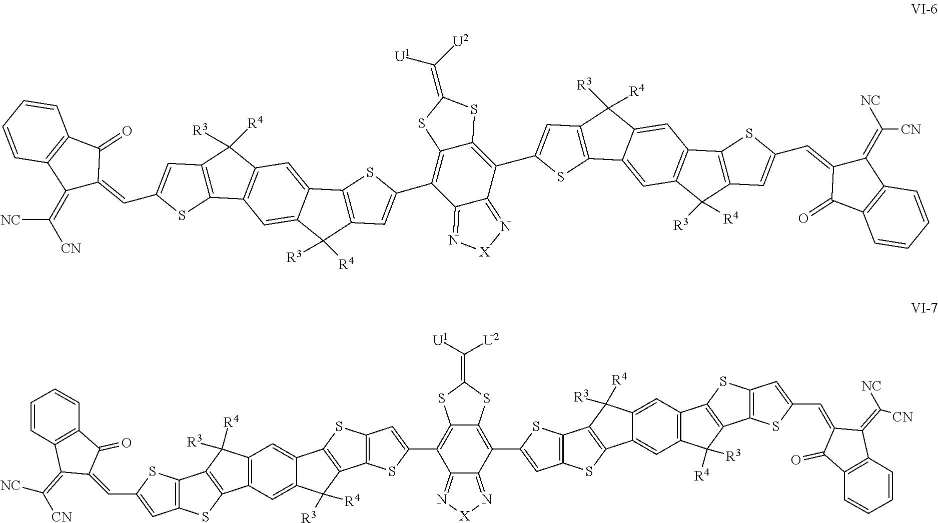

wherein G, X, U¹, U², R³ and R⁴ are as defined above, and U¹ and U² preferably denote —COO or CN.

Further preferred compounds of formula VI are selected from formula VI1

R^{T1}—U*—R^{T2} VI1

wherein R^{T1} and R^{T2} have the meanings given above and below, and preferably denote H, F, R or OR or an electron withdrawing group, and U* is a unit selected from the subformulae P1-P18 and P1a-P12b above wherein n is 1.

Another preferred embodiment of the present invention relates to a compound which is a monomer of formula V1 or V2

R²³—(Ar¹)ₐ—U—(Ar²)_{b}—(Ar³)_{c}—(Ar⁴)_{d}—R²⁴ V1

R²³—(Ar¹)ₐ—(Ar²)_{b}—U—(Ar³)_{c}—(Ar⁴)_{d}—R²⁴ V2

wherein U, Ar¹⁻⁴, a, b, c and d have the meanings of formula II1, or one of the preferred meanings as described above and below, and R²³ and R²⁴ are independently of each other selected from the group consisting of H, which is preferably an activated C—H bond, Cl, Br, I, O-tosylate, O-triflate, O-mesylate, O-nonaflate, —SiMe₂F, —SiMeF₂, —O—SO₂Z¹, —B(OZ²)₂, —CZ³═C(Z³)₂, —C≡CH, —C≡CSi(Z¹)₃, —ZnX⁰ and —Sn(Z⁴)₃, wherein X⁰ is halogen, Z¹⁻⁴ are selected from the group consisting of alkyl and aryl, preferably C₁₋₁₀ alkyl and C₆₋₁₂ aryl, each being optionally substituted, and two groups Z² may also form a cycloboronate group having 2 to 20 C atoms together with the B- and O-atoms, and wherein at least one of R²³ and R²⁴ is different from H, and preferably both of R²³ and R²⁴ are different from H.

Very preferred are monomers of formula V1 and V2 and their subformulae wherein a+b+c+d≥1.

Further preferred are monomers of formula V1 and its subformulae wherein a+b+c+d=0.

Further preferred are monomers of formula V1 and V2 and their subformulae wherein R²³ and R²⁴ are selected from Br, —B(OZ²)₂ and Sn(Z⁴)₃.

Further preferred are monomers selected from the following subformulae

R²³—Ar¹—U—Ar²—R²⁴ V1a

R²³—U—R²⁴ V1b

R²³—Ar¹—U—R²⁴ V1c

R²³—U—Ar²—R²⁴ V1d

wherein U, Ar¹, Ar², R²³ and R²⁴ are as defined in formula V1.

Very preferred are monomers of formula V1 and V2 and their subformulae wherein R²³ and R²⁴ are selected from Br, B(OZ²)₂ and Sn(Z⁴)₃.

Further preferred are monomers of formulae V1, V2, V1a-V1d and their subformulae wherein Ar¹ and/or Ar² are selected from the following groups
a) the group consisting of the formulae D1-D145, very preferably of the formulae D1, D7, D10, D11, D19, D22, D29, D30, D35, D36, D37, D44, D55, D84, D87, D88, D89, D93, D94, D106, D111, D139, D140, D141 and D150,
b) the group consisting of the formulae A1-A98, very preferably of the formulae A1, A6, A7, A15, A16, A20, A36, A74, A84, A88, A92, A98 and A103,
c) the group consisting of the formulae Sp1-Sp18, very preferably of the formulae Sp1, Sp2, Sp6, Sp10, Sp11, Sp12, Sp13 and Sp14.

Further preferred are monomers of formula V3

R²³—U*—R²⁴ V3

wherein R²³ and R²⁴ have the meanings given above and below, and preferably denote Br, B(OZ²)₂ or Sn(Z⁴)₃, and U* is a unit selected from the subformulae P1-P18 and P1a-P12b above wherein n is 1.

Further preferred units, monomers, oligomers, polymers and small molecules of formulae I, II1, II2, II3, III, III1-III8, P1-P43, P1a-P12b, IV, V1, V2, V3, VI, VI1 and their subformulae are selected from the following embodiments, including any combination thereof:
X is S,
X is O,
X is CH═CH,
Y is N,
Y is CH,
R¹⁻² are selected from alkyl, alkoxy or thiaalkyl, all of which are straight-chain or branched, have 1 to 25, preferably 1 to 18 C atoms, and are optionally fluorinated,
R¹⁻² are selected from —C(═O)—Rⁿ, —C(═O)—ORⁿ, —C(═O)—NHRⁿ and —C(═O)—NRⁿR^{m}, wherein R^{m} and Rⁿ are independently of each other straight-chain or branched alkyl with 1 to 25, preferably 1 to 18 C atoms that is optionally fluorinated,
R¹⁻² are cyclic alkyl with 4 to 20 ring atoms, wherein one or more CH₂ groups are optionally replaced by O, S, NR⁰, C(═O), (C═S), CY¹═CY² or CR⁰═CR⁰⁰, and which is unsubstituted or substituted by one or more groups L as defined in formula I,
R¹⁻² are selected from the group consisting of aryl, heteroaryl, aryloxy, heteroaryloxy, arylalkyl and heteroarylalkyl, each of which has 4 to 20 ring atoms and optionally contains fused rings and is unsubstituted or substituted by one or more groups L as defined in formula I,
R³⁻⁶ are selected from alkyl, alkoxy or thiaalkyl, all of which are straight-chain or branched, have 1 to 25, preferably 1 to 18 C atoms, and are optionally fluorinated,
R³⁻⁶ are selected from —C(═O)—Rⁿ, —C(═O)—ORⁿ, —C(═O)—NHRⁿ and —C(═O)—NRⁿR^{m}, wherein R^{m} and Rⁿ are independently of each other straight-chain or branched alkyl with 1 to 25, preferably 1 to 18 C atoms that is optionally fluorinated,
R³⁻⁶ are cyclic alkyl with 4 to 20 ring atoms, wherein one or more CH₂ groups are optionally replaced by O, S, NR⁰, C(═O), (C═S), CY¹═CY² or CR⁰═CR⁰⁰, and which is unsubstituted or substituted by one or more groups L as defined in formula I,
R³⁻⁶ are selected from the group consisting of aryl, heteroaryl, aryloxy, heteroaryloxy, arylalkyl and heteroarylalkyl, each of which has 4 to 20 ring atoms and optionally contains fused rings and is unsubstituted or substituted by one or more groups L as defined in formula I,
R is straight-chain or branched alkyl with 1 to 25, preferably 1 to 18 C atoms which is optionally fluorinated,
R is cyclic alkyl with 4 to 20 ring atoms, wherein one or more CH₂ groups are optionally replaced by O, S, NR⁰, C(═O), (C═S), CY¹═CY² or CR⁰═CR⁰⁰, and which is unsubstituted or substituted by one or more groups L as defined in formula I,
R is aryl, heteroaryl, arylalkyl or heteroarylalkyl, each of which has 4 to 20 ring atoms, optionally contains fused rings, and is unsubstituted or substituted by one or more groups L as defined in formula I,
U¹ and U² denote CN,
U¹ and U² denote C(═O)R or C(═O)OR, wherein R has one of the meanings given above and below, and is preferably straight-chain or branched alkyl with 1 to 25, preferably 1 to 18 C atoms which is optionally fluorinated,
L is selected from alkyl, alkoxy or thiaalkyl, all of which are straight-chain or branched, have 1 to 25, preferably 1 to 18 C atoms, and are optionally fluorinated,
L is selected from —C(═O)—Rⁿ, —C(═O)—ORⁿ, —C(═O)—NHRⁿ and —C(═O)—NRⁿR^{m}, wherein R^{m} and Rⁿ are independently of each other straight-chain or branched alkyl with 1 to 25, preferably 1 to 18 C atoms that is optionally fluorinated,
L is halogen, preferably F or Cl,
L is CN, F or Cl,
R²¹ and R²² are selected from H, C₁₋₂₀ alkyl, or optionally substituted C₆₋₁₂ aryl or C₂₋₁₀ heteroaryl, very preferably H or phenyl,
R²³ and R²⁴ denote Br, B(OZ²)₂ or Sn(Z⁴)₃, wherein Z² and Z⁴ are as defined in formula V1.

The polymer according to the present invention can be synthesized according to or in analogy to methods that are known to the skilled person and are described in the literature. Other methods of preparation can be taken from the examples.

The polymers according to the present invention can be prepared for example by copolymerising one or more monomers of formula V1, V2 or V1a-V1d with each other or with one or monomers of the following formulae in an aryl-aryl coupling reaction

R²³—Ar¹—R²⁴ MI

R²³—Ar²—R²⁴ MII

R²³—Ar³—R²⁴ MIII

R²³—Ar⁴—R²⁴ MIV

wherein Ar¹⁻⁴, R²³ and R²⁴ have the meanings given in formula II2 and V1 or one of the preferred meanings given above and below.

For example, the polymer can be suitably prepared by aryl-aryl coupling reactions, such as Yamamoto coupling, C—H activation coupling, Suzuki coupling, Stille coupling, Sonogashira coupling, Heck coupling or Buchwald coupling. Suzuki coupling, Stille coupling and Yamamoto coupling are especially preferred. The monomers which are polymerised to form the repeat units of the polymers can be prepared according to methods which are known to the person skilled in the art.

Preferably the polymer is prepared from monomers selected from formulae V1, V2, V3, V1a-d and MI-MIV as described above.

Another aspect of the invention is a process for preparing a polymer by coupling one or more identical or different monomers selected from formulae V1, V2, V1a-d with each other and/or with one or more co-monomers, preferably selected from formulae MI-MIV, in a polymerisation reaction, preferably in an aryl-aryl coupling reaction.

Preferred aryl-aryl coupling and polymerisation methods used in the processes described above and below are Yamamoto coupling, Kumada coupling, Negishi coupling, Suzuki coupling, Stille coupling, Sonogashira coupling, Heck coupling, C—H activation coupling, Ullmann coupling or Buchwald coupling. Especially preferred are Suzuki coupling, Negishi coupling, Stille coupling and Yamamoto coupling. Suzuki coupling is described for example in WO 00/53656 A1. Negishi coupling is described for example in J. Chem. Soc., Chem. Commun., 1977, 683-684. Yamamoto coupling is described in for example in T. Yamamoto et al., Prog. Polym. Sci., 1993, 17, 1153-1205, or WO 2004/022626 A1. Stille coupling is described for example in Z. Bao et al., J. Am. Chem. Soc., 1995, 117, 12426-12435. C—H activation is described for example for example in M. Leclerc et al, Angew. Chem. Int. Ed. 2012, 51, 2068-2071. For example, when using Yamamoto coupling, monomers having two reactive halide groups are preferably used. When using Suzuki coupling, monomers having two reactive boronic acid or boronic acid ester groups or two reactive halide groups are preferably used. When using Stille coupling, monomers having two reactive stannane groups or two reactive halide groups are preferably used. When using Negishi coupling, monomers having two reactive organozinc groups or two reactive halide groups are preferably used. When synthesizing a linear polymer by C—H activation polymerisation, preferably a monomer as described above is used wherein at least one reactive group is an activated hydrogen bond.

Preferred catalysts, especially for Suzuki, Negishi or Stille coupling, are selected from Pd(0) complexes or Pd(II) salts. Preferred Pd(0) complexes are those bearing at least one phosphine ligand such as Pd(Ph₃P)₄. Another preferred phosphine ligand is tris(ortho-tolyl)phosphine, i.e. Pd(o-Tol₃P)₄. Preferred Pd(II) salts include palladium acetate, i.e. Pd(Oac)₂ or trans-di(μ-acetato)-bis[o-(di-o-tolylphosphino)benzyl]dipalladium(II). Alternatively the Pd(0) complex can be prepared by mixing a Pd(0) dibenzylideneacetone complex, for example tris(dibenzyl-ideneacetone)dipalladium(0), bis(dibenzylideneacetone)palladium(0), or Pd(II) salts e.g. palladium acetate, with a phosphine ligand, for example triphenylphosphine, tris(ortho-tolyl)phosphine, tris(o-methoxyphenyl)phosphine or tri(tert-butyl)phosphine. Suzuki polymerisation is performed in the presence of a base, for example sodium carbonate, potassium carbonate, cesium carbonated, lithium hydroxide, potassium phosphate or an organic base such as tetraethylammonium carbonate or tetraethylammonium hydroxide. Yamamoto polymerisation employs a Ni(0) complex, for example bis(1,5-cyclooctadienyl) nickel(0).

Suzuki, Stille or C—H activation coupling polymerisation may be used to prepare homopolymers as well as statistical, alternating and block random copolymers. Statistical, random block copolymers or block copolymers can be prepared for example from the above monomers, wherein one of the reactive groups is halogen and the other reactive group is a C—H activated bond, boronic acid, boronic acid derivative group or and alkylstannane.

The synthesis of statistical, alternating and block copolymers is described in detail for example in WO 03/048225 A2 or WO 2005/014688 A2.

As alternatives to halogen as described above, leaving groups of formula —O—SO₂Z¹ can be used wherein Z¹ is as defined above. Particular examples of such leaving groups are tosylate, mesylate and triflate.

Preferred polymerisation conditions lead to alternating polymers which are particularly preferred for OTFT application, whereas statistical block co-polymers are prepared preferably for OPV and OPD application. Preferred polycondensation are Suzuki coupling, Stille coupling, Sonogashira coupling, Heck coupling or Buchwald coupling, Negishi coupling or C—H activation coupling where the first set of reactive groups is composed of —Cl, —Br, —I, O-tosylate, O-triflate, O-mesylate and O-nonaflate and the second set of reactive groups is composed of —H, —SiR₂F, —SiRF₂, —B(OR)₂, —CR═CHR′, —C≡CH, —ZnX, —MgX and —Sn(R₃). If a Yamamoto coupling reaction is used to prepare the polymer, the reactive monomer ends are both composed independently of —Cl, —Br, —I, O-tosylate, O-triflate, O-mesylate and O-nonaflate.

Suitable and preferred methods for preparing compounds according to the present invention are illustrated in the reaction schemes below, wherein the individual radicals are as defined above. The compounds not shown, e.g. wherein in the units of formula I X is S, or Y is O, S or CU¹U², can be made in analogy thereto.

The core DTBTz and DTQ units can be synthesized as schematically illustrated in Scheme 1 starting from a common intermediate, 3,6-dibromo-4,5-dichlorophenylene-1,2-diamine, which can be prepared in high yield using a novel bromination method of 4,5-dichlorophenylene-1,2-diamine as disclosed in this invention. In this method, hydrobromic acid is used to protonate 4,5-dichlorophenylene-1,2-diamine prior to the addition of bromine. This process protects the phenylenediamine from being oxidised by bromine or other bromination agents. Using standard methods, 3,6-dibromo-4,5-dichlorophenylene-1,2-diamine can be ring-closed to the corresponding 4,7-dibromo-5,6-dichloro-2,1,3-benzothiadiazole and 5,8-dibromo-6,7-dichloro-quinoxaline.

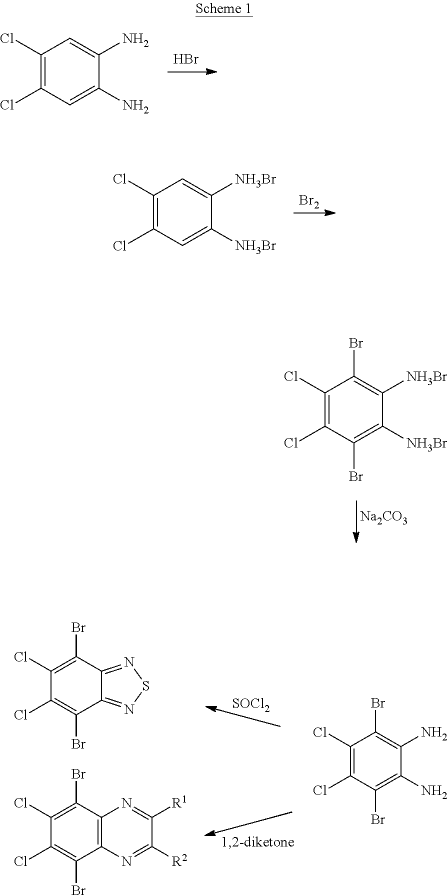

As exemplarily illustrated in Scheme 2, cross-coupling of 4,7-dibromo-5,6-dichloro-2,1,3-benzothiadiazole and 5,8-dibromo-6,7-dichloro-quinoxaline with arylboronic acids, arylboronic esters, arylstannanes, aryl Grignard reagents or arylzink halide to afford n-extended 4,7-diaryl-5,6-dichloro-2,1,3-benzothiadiazole and 5,8-diaryl-6,7-dichloro-quinoxaline. The ortho chlorine atoms can in turn be replaced by reacting with disodium methanedithiolate to yield the corresponding DTBTz and DTQ units.

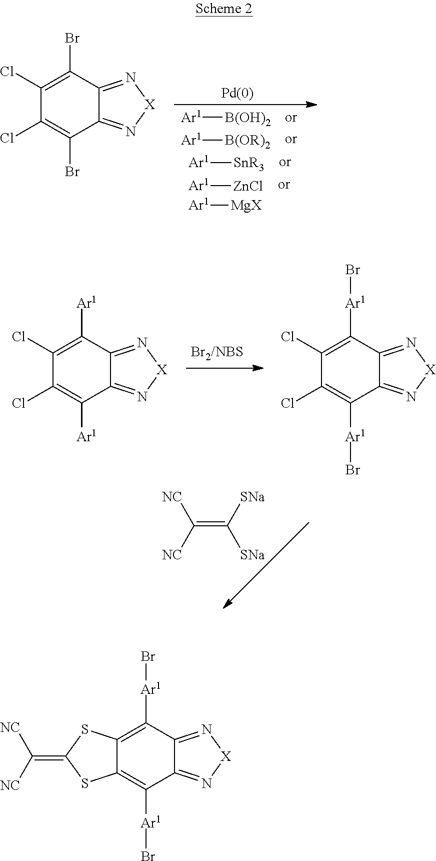

As exemplarily illustrated in Scheme 3, unbrominated DTBTz and DTQ units are synthesised from 5,6-dichloro-2,1,3-benzothiadiazole and 6,7-dichloro-quinoxaline directly.

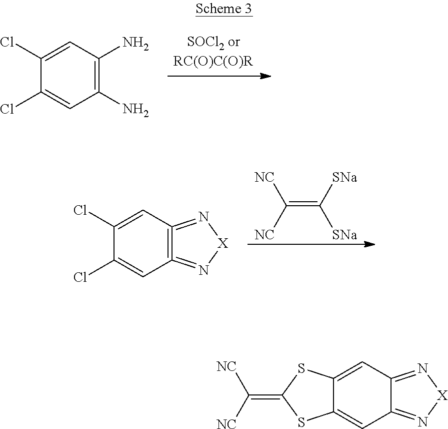

Some preferred polymerization reactions are exemplarily illustrated represented in Scheme 4. Conjugated polymers and co-polymers, including alternating co-polymers and statistical block co-polymers can be made by the methods described above. Particularly, conjugated polymer can be made by Pd catalysed direct arylation polymerisation with a dibromo counterpart (M. Wakioka, et al., Macromol., 2015, 48, 8382) or Pd catalysed polycondensations methods such as Yamamoto reaction (Yamamoto et al., Bull., Chem. Soc. Jpn., 1978, 51(7), 2091; Yamamo to et al., Macromolecules, 1992, 25(4), 1214), Suzuki-Miyaura reaction (Miyaura et al., Chem. Rev., 1995, 95, 2457) and Stille reaction (Bao et al., J. Am., Chem., Soc., 1995, 117(50), 12426) using the terminally brominated derivatives.

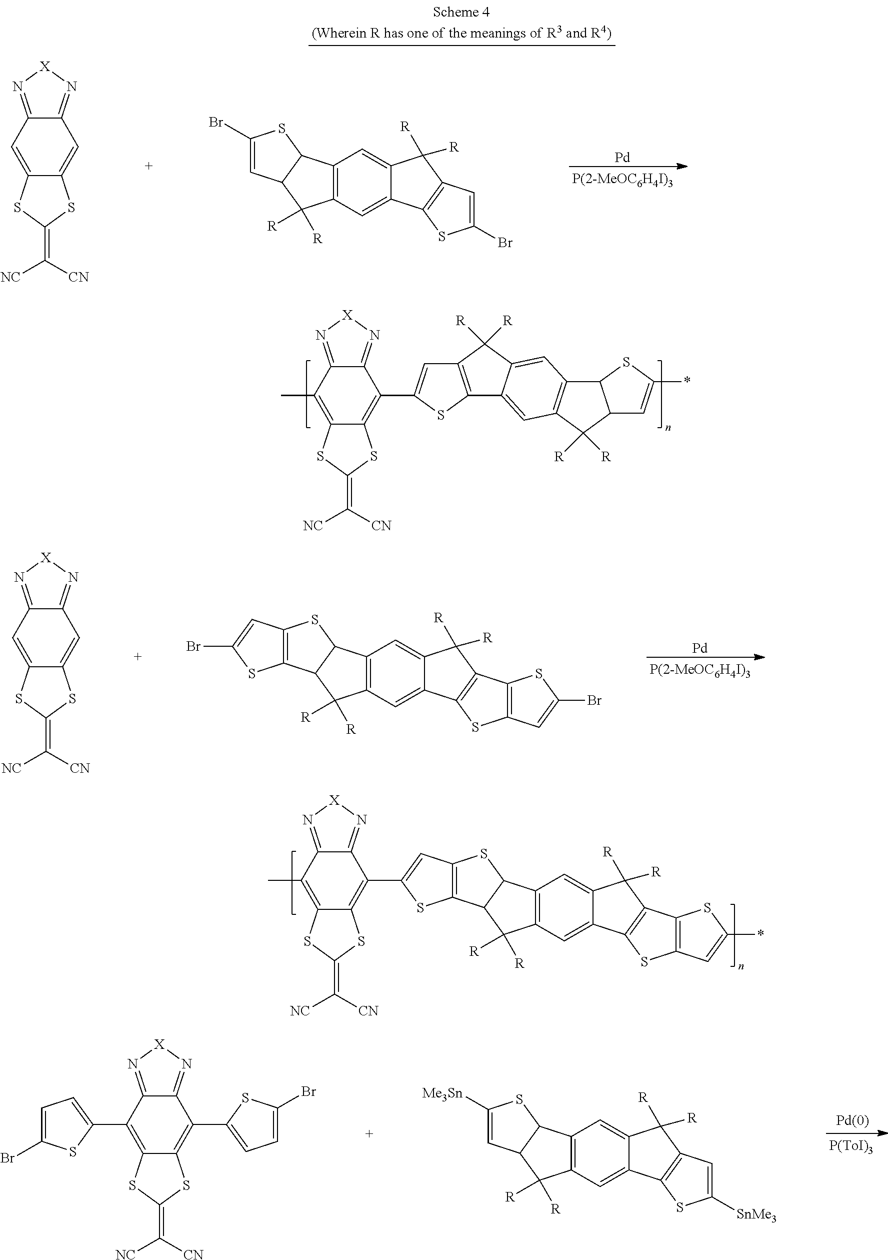

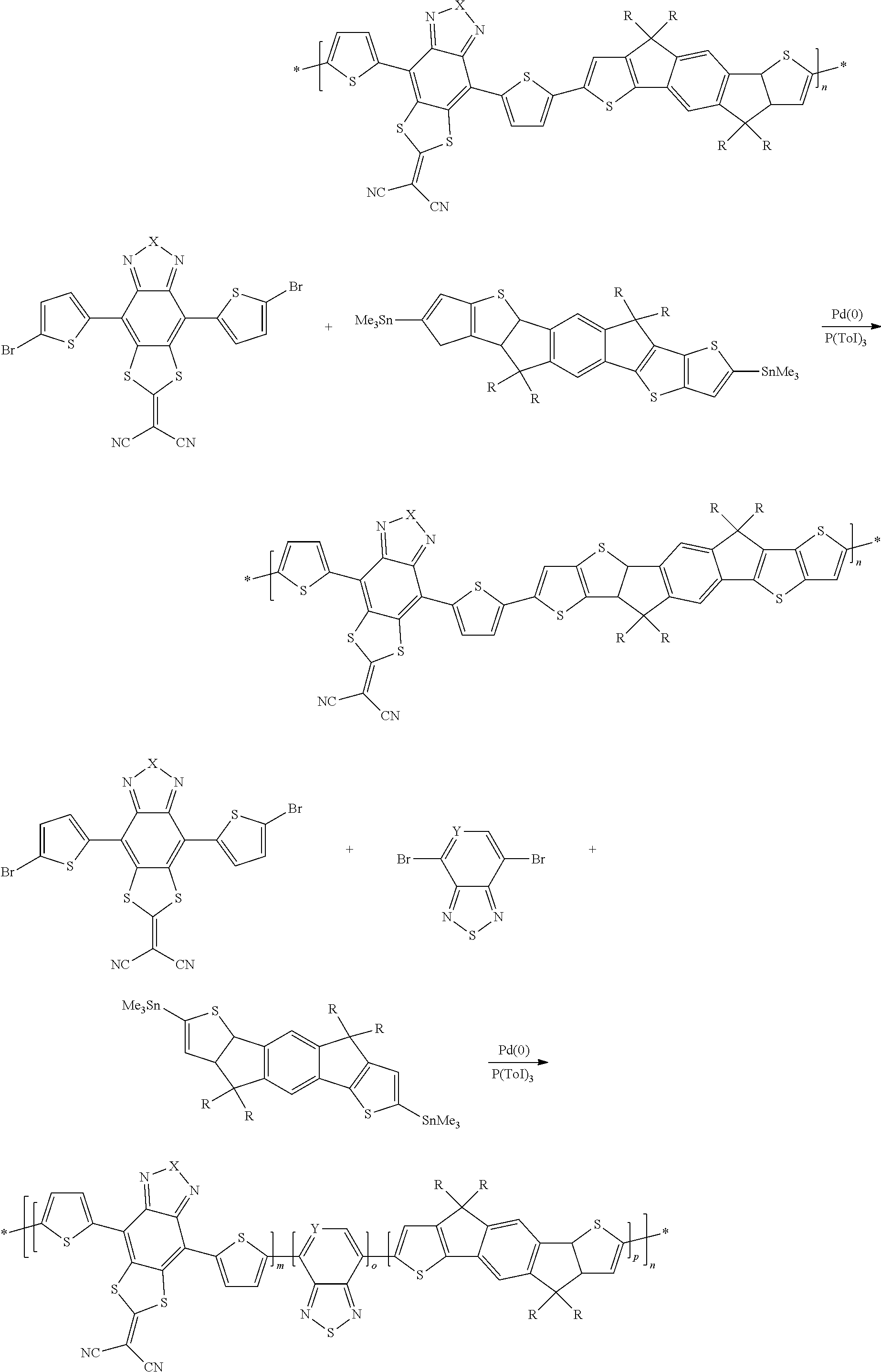

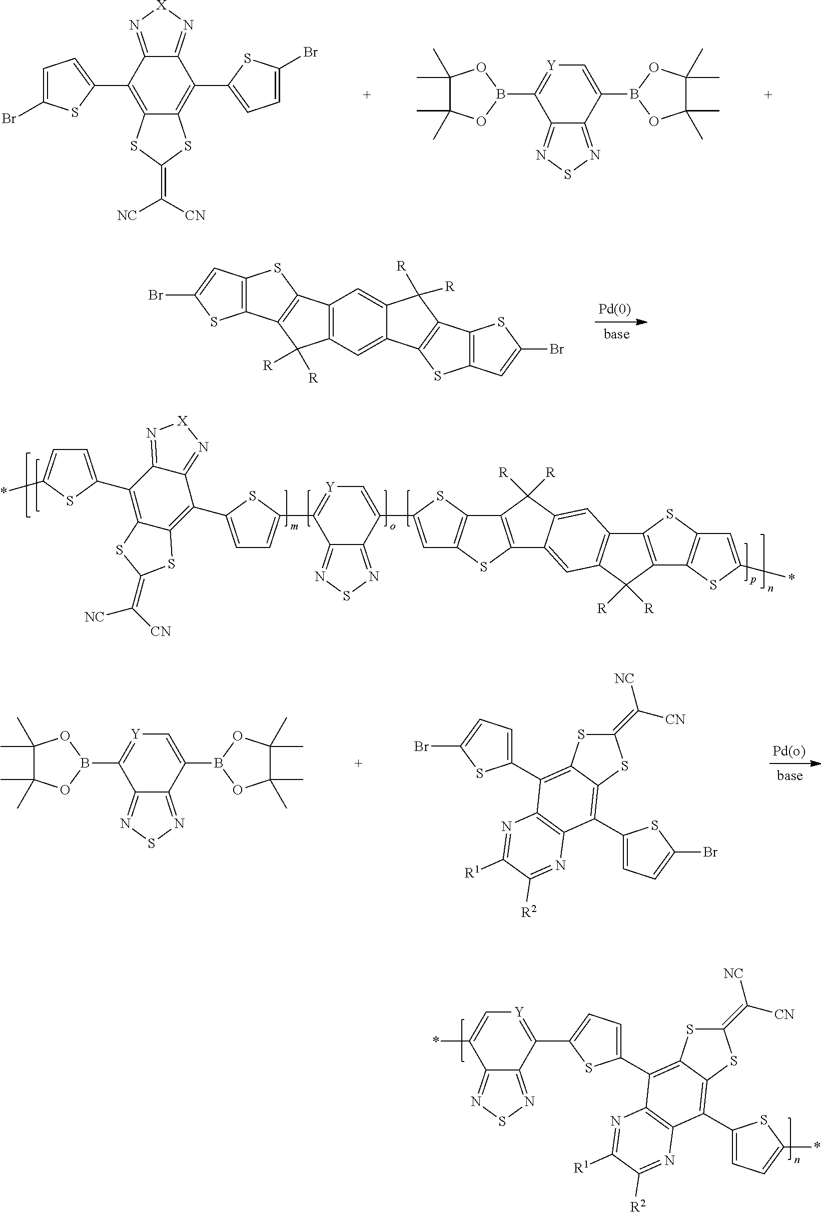

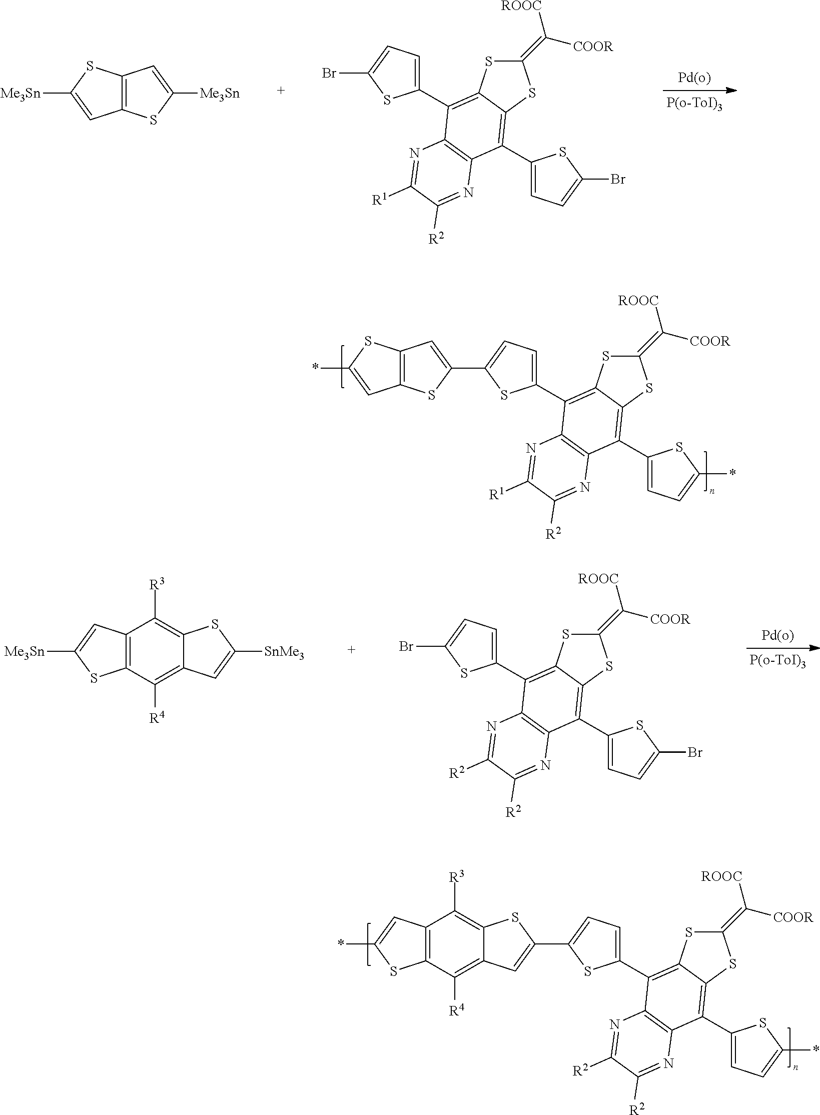

The novel polymers shown in Scheme 4 are a further subject of the invention.

The synthesis of oligomers and small molecules based on the DTBTz and DTQ cores is exemplarily illustrated in Scheme 5. Alternatively these compounds can be obtained via a convergent synthesis strategy as shown in Scheme 6.

Therein Y and R¹ are as defined in formula I, X₁═Br and X₂═SnR′₃ or B(OR′)₂ or X₁═SnR′₃ and X₂═Br or X₁═B(OR′)₂ and X₂═Br, Ar₁₋₈ correspond to Ar¹⁻⁸ as defined in formula VI, and Ar₅—Ar₆—Ar₇—Ar₈—R²_{end} is identical to Ar₄—Ar₃—Ar₂—Ar₁—R¹_{end}, and R¹_{end} and R²_{end} correspond to R^{T1} and R^{T2} in formula VI, and U has one of the meanings of U¹ and U² as given above.

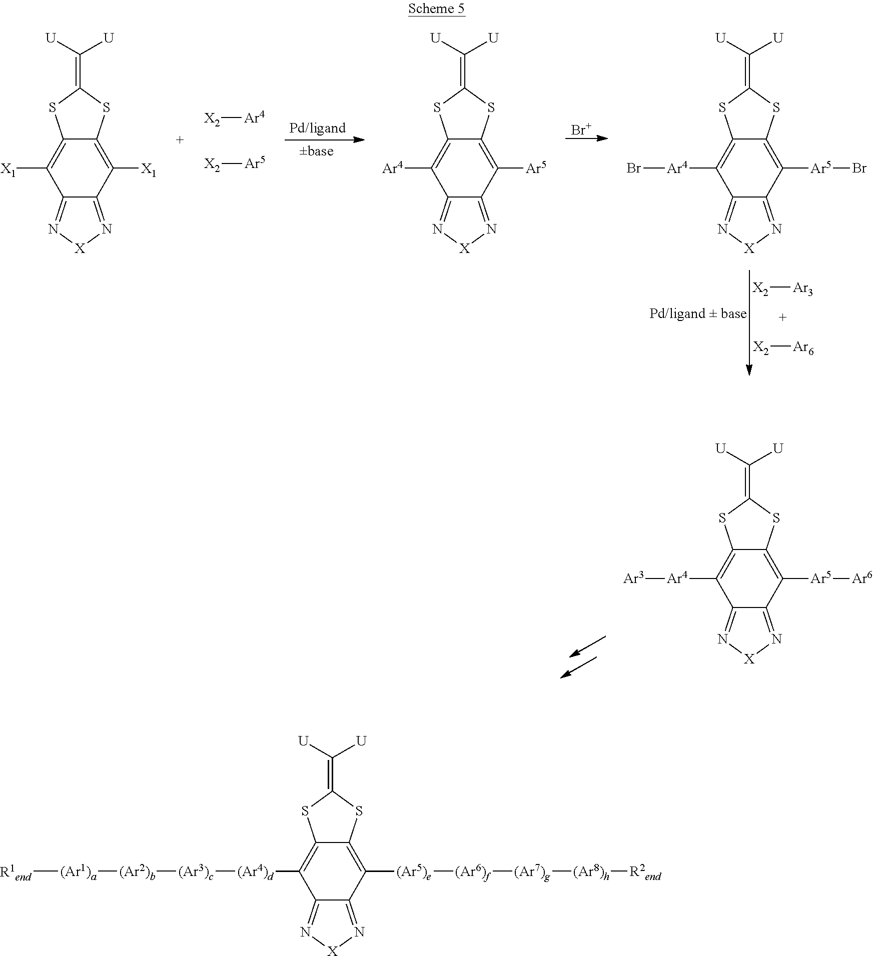

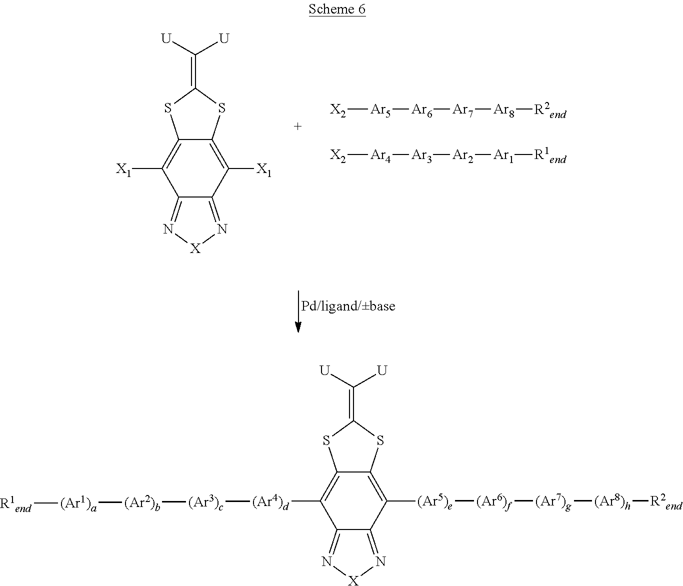

Alternatively asymmetric small molecules based on DTBTz and DTQ can be obtained via a convergent synthesis strategy as exemplarily illustrated in Scheme 7, wherein the individual radicals are as defined in Scheme 5 and 6.

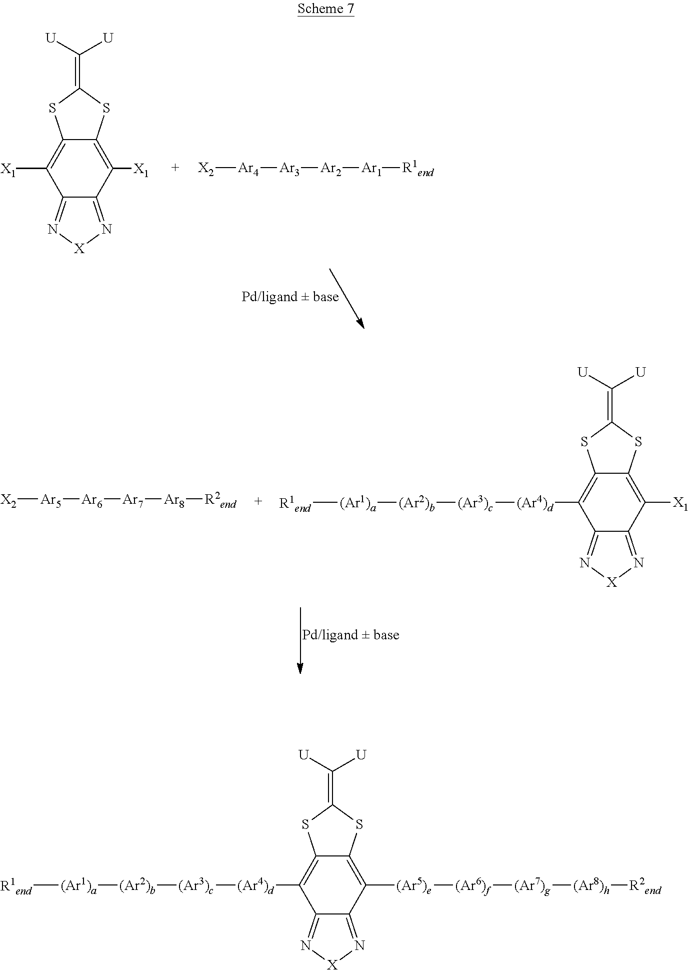

The synthesis of asymmetric compounds containing multiple DTBTz and DTQ units via a convergent synthesis strategy is exemplarily illustrated in Scheme 8, wherein the individual radicals are as defined in Scheme 5, and 1<n≤3.

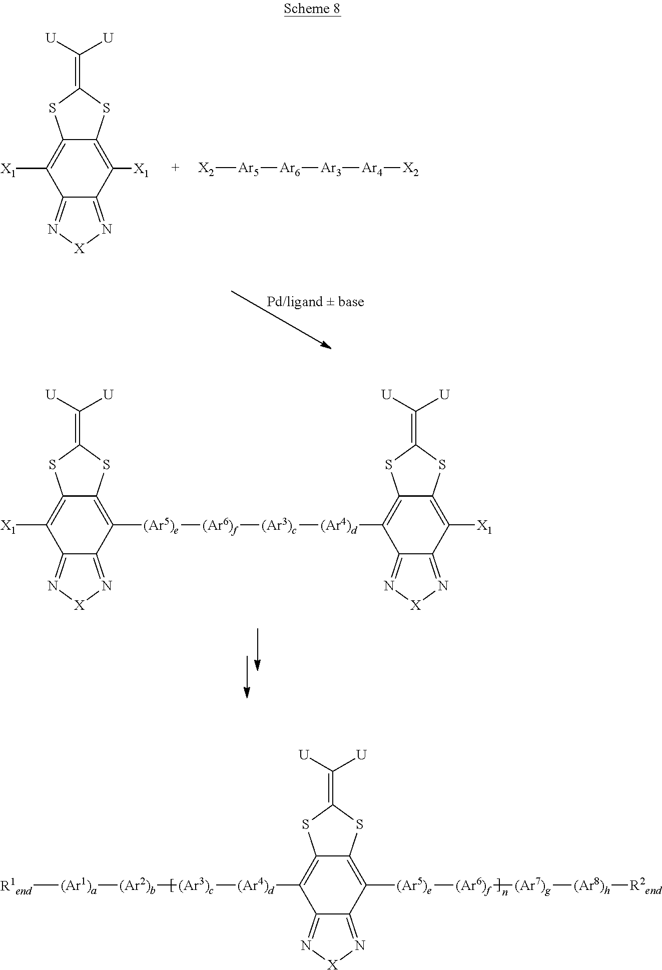

Further substitution can be added to the DTBTz and DTQ core at the R^{1,2}_{end} substitution after the DTBTz and DTQ core compounds have been prepared as exemplarily illustrated in Scheme 9, wherein the individual radicals are as defined in Scheme 6.

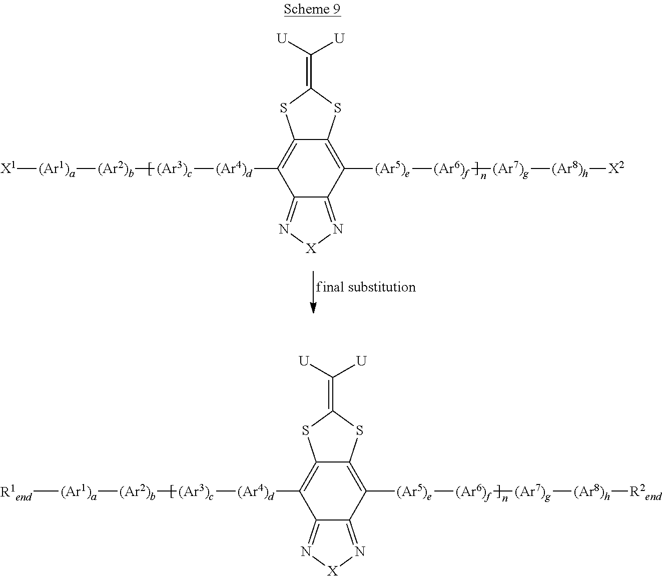

The novel methods of preparing a compound, monomer or polymer as described above and below, and the novel monomers and intermediates used therein, are further aspects of the invention.

The compounds according to the present invention can also be used in compositions or polymer blends, for example together with small molecules or other polymers having charge-transport, semiconducting, electrically conducting, photoconducting and/or light-emitting semiconducting properties, or for example with polymers having hole blocking, electron blocking properties for use as interlayers, charge blocking layers, charge transporting layer in OLED devices, OPV devices or perovskite based solar cells.

Small molecules according to the present invention which contain one or more electron withdrawing groups can also be used as n-type semiconductors. For example they can be used as replacement of, or in addition to, fullerenes, especially in mixtures or blends of p-type and n-type semiconductors for use in OPV or OPD devices. Preferred compounds for use as n-type semiconductors are those of formula VI or their subformulae, wherein R^{T1} and/or R^{T2} denote or contain an electron withdrawing group.

Another aspect of the invention relates to a composition, which may also be a polymer blend, comprising one or more compounds according to the present invention and one or more small molecule compounds and/or polymers having one or more of a charge-transport, semiconducting, electrically conducting, photoconducting, hole blocking and electron blocking property.

These compositions can be prepared by conventional methods that are described in prior art and known to the skilled person. Typically the compounds are mixed with each other or dissolved in suitable solvents and the solutions combined.

Another aspect of the invention relates to a formulation comprising one or more polymers, polymer blends or compositions as described above and below and one or more organic solvents.

Preferred solvents are aliphatic hydrocarbons, chlorinated hydrocarbons, aromatic hydrocarbons, ketones, ethers and mixtures thereof. Additional solvents which can be used include 1,2,4-trimethylbenzene, 1,2,3,4-tetra-methyl benzene, pentylbenzene, mesitylene, cumene, cymene, cyclohexylbenzene, diethylbenzene, tetralin, decalin, 2,6-lutidine, 2-fluoro-m-xylene, 3-fluoro-o-xylene, 2-chlorobenzotrifluoride, N,N-dimethylformamide, 2-chloro-6-fluorotoluene, 2-fluoroanisole, anisole, 2,3-dimethylpyrazine, 4-fluoroanisole, 3-fluoroanisole, 3-trifluoro-methylanisole, 2-methylanisole, phenetol, 4-methylanisole, 3-methylanisole, 4-fluoro-3-methylanisole, 2-fluorobenzonitrile, 4-fluoroveratrol, 2,6-dimethylanisole, 3-fluorobenzo-nitrile, 2,5-dimethylanisole, 2,4-dimethylanisole, benzonitrile, 3,5-dimethyl-anisole, N,N-dimethylaniline, ethyl benzoate, 1-fluoro-3,5-dimethoxy-benzene, 1-methylnaphthalene, N-methylpyrrolidinone, 3-fluorobenzo-trifluoride, benzotrifluoride, dioxane, trifluoromethoxy-benzene, 4-fluorobenzotrifluoride, 3-fluoropyridine, toluene, 2-fluoro-toluene, 2-fluorobenzotrifluoride, 3-fluorotoluene, 4-isopropylbiphenyl, phenyl ether, pyridine, 4-fluorotoluene, 2,5-difluorotoluene, 1-chloro-2,4-difluorobenzene, 2-fluoropyridine, 3-chlorofluoro-benzene, 1-chloro-2,5-difluorobenzene, 4-chlorofluorobenzene, chloro-benzene, o-dichlorobenzene, 2-chlorofluorobenzene, p-xylene, m-xylene, o-xylene or mixture of o-, m-, and p-isomers. Solvents with relatively low polarity are generally preferred. For inkjet printing solvents and solvent mixtures with high boiling temperatures are preferred. For spin coating alkylated benzenes like xylene and toluene are preferred.

Examples of especially preferred solvents include, without limitation, dichloromethane, trichloromethane, chlorobenzene, o-dichlorobenzene, tetrahydrofuran, anisole, 2,4-dimethylanisole, 1-methylnaphthalene, morpholine, toluene, o-xylene, m-xylene, p-xylene, 1,4-dioxane, acetone, methylethylketone, 1,2-dichloroethane, 1,1,1-trichloroethane, 1,1,2,2-tetrachloroethane, ethyl acetate, n-butyl acetate, N,N-dimethylformamide, dimethylacetamide, dimethylsulfoxide, 1,5-dimethyltetraline, propiophenone, acetophenone, tetraline, 2-methylthiophene, 3-methylthiophene, decaline, indane, methyl benzoate, ethyl benzoate, mesitylene and/or mixtures thereof.

The concentration of the polymers in the solution is preferably 0.1 to 10% by weight, more preferably 0.5 to 5% by weight. Optionally, the solution also comprises one or more binders to adjust the rheological properties, as described for example in WO 2005/055248 A1.

After the appropriate mixing and ageing, solutions are evaluated as one of the following categories: complete solution, borderline solution or insoluble. The contour line is drawn to outline the solubility parameter-hydrogen bonding limits dividing solubility and insolubility. ‘Complete’ solvents falling within the solubility area can be chosen from literature values such as published in “Crowley, J. D., Teague, G. S. Jr and Lowe, J. W. Jr., Journal of Paint Technology, 1966, 38 (496), 296”. Solvent blends may also be used and can be identified as described in “Solvents, W. H. Ellis, Federation of Societies for Coatings Technology, p 9-10, 1986”. Such a procedure may lead to a blend of ‘non’ solvents that will dissolve both the polymers of the present invention, although it is desirable to have at least one true solvent in a blend.

The compounds according to the present invention can also be used in patterned OSC layers in the devices as described above and below. For applications in modern microelectronics it is generally desirable to generate small structures or patterns to reduce cost (more devices/unit area), and power consumption. Patterning of thin layers comprising a polymer according to the present invention can be carried out for example by photolithography, electron beam lithography or laser patterning.

For use as thin layers in electronic or electrooptical devices the compounds, compositions or formulations according to the present invention may be deposited by any suitable method. Liquid coating of devices is more desirable than vacuum deposition techniques. Solution deposition methods are especially preferred. The formulations of the present invention enable the use of a number of liquid coating techniques. Preferred deposition techniques include, without limitation, dip coating, spin coating, ink jet printing, nozzle printing, letter-press printing, screen printing, gravure printing, doctor blade coating, roller printing, reverse-roller printing, offset lithography printing, dry offset lithography printing, flexographic printing, web printing, spray coating, curtain coating, brush coating, slot dye coating or pad printing.

Ink jet printing is particularly preferred when high resolution layers and devices needs to be prepared. Selected formulations of the present invention may be applied to prefabricated device substrates by ink jet printing or microdispensing. Preferably industrial piezoelectric print heads such as but not limited to those supplied by Aprion, Hitachi-Koki, InkJet Technology, On Target Technology, Picojet, Spectra, Trident, Xaar may be used to apply the organic semiconductor layer to a substrate. Additionally semi-industrial heads such as those manufactured by Brother, Epson, Konica, Seiko Instruments Toshiba TEC or single nozzle microdispensers such as those produced by Microdrop and Microfab may be used.

In order to be applied by ink jet printing or microdispensing, the polymers should be first dissolved in a suitable solvent. Solvents must fulfil the requirements stated above and must not have any detrimental effect on the chosen print head. Additionally, solvents should have boiling points >100° C., preferably >140° C. and more preferably >150° C. in order to prevent operability problems caused by the solution drying out inside the print head. Apart from the solvents mentioned above, suitable solvents include substituted and non-substituted xylene derivatives, di-C₁₋₂-alkyl formamide, substituted and non-substituted anisoles and other phenol-ether derivatives, substituted heterocycles such as substituted pyridines, pyrazines, pyrimidines, pyrrolidinones, substituted and non-substituted N,N-di-C₁₋₂-alkylanilines and other fluorinated or chlorinated aromatics.

A preferred solvent for depositing a compound according to the present invention by ink jet printing comprises a benzene derivative which has a benzene ring substituted by one or more substituents wherein the total number of carbon atoms among the one or more substituents is at least three. For example, the benzene derivative may be substituted with a propyl group or three methyl groups, in either case there being at least three carbon atoms in total. Such a solvent enables an ink jet fluid to be formed comprising the solvent with the compound or polymer, which reduces or prevents clogging of the jets and separation of the components during spraying. The solvent(s) may include those selected from the following list of examples: dodecylbenzene, 1-methyl-4-tert-butylbenzene, terpineol, limonene, isodurene, terpinolene, cymene, diethylbenzene. The solvent may be a solvent mixture, that is a combination of two or more solvents, each solvent preferably having a boiling point >100° C., more preferably >140° C. Such solvent(s) also enhance film formation in the layer deposited and reduce defects in the layer.

The ink jet fluid (that is mixture of solvent, binder and semiconducting compound) preferably has a viscosity at 20° C. of 1-100 mPa·s, more preferably 1-50 mPa·s and most preferably 1-30 mPa·s.

The compounds, compositions and formulations according to the present invention can additionally comprise one or more further components or additives selected for example from surface-active compounds, lubricating agents, wetting agents, dispersing agents, hydrophobing agents, adhesive agents, flow improvers, defoaming agents, deaerators, diluents which may be reactive or non-reactive, auxiliaries, colourants, dyes or pigments, sensitizers, stabilizers, nanoparticles or inhibitors.

The compounds and compositions according to the present invention are useful as charge transport, semiconducting, electrically conducting, photoconducting or light emitting material in optical, electrooptical, electronic, electroluminescent or photoluminescent components or devices. In these devices, a compound or composition of the present invention is typically applied as a thin layer or film.

Thus, the present invention also provides the use of the compound, composition or layer in an electronic device. The formulation may be used as a high mobility semiconducting material in various devices and apparatus. The formulation may be used, for example, in the form of a semiconducting layer or film. Accordingly, in another aspect, the present invention provides a semiconducting layer for use in an electronic device, the layer comprising a polymer, composition or polymer blend according to the invention. The layer or film may be less than about 30 microns. For various electronic device applications, the thickness may be less than about 1 micron thick. The layer may be deposited, for example on a part of an electronic device, by any of the aforementioned solution coating or printing techniques.

The invention additionally provides an electronic device comprising a polymer, polymer blend, composition or organic semiconducting layer according to the present invention. Especially preferred devices are OFETs, TFTs, ICs, logic circuits, capacitors, RFID tags, OLEDs, OLETs, OPEDs, OPVs, OPDs, solar cells, dye-sensitized solar cells (DSSC), perovskite-based solar cells, laser diodes, photoconductors, photodetectors, electrophotographic devices, electrophotographic recording devices, organic memory devices, sensor devices, charge injection layers, Schottky diodes, planarising layers, antistatic films, conducting substrates and conducting patterns.

Especially preferred electronic device are OFETs, OLEDs, OPV and OPD devices, in particular OPD and bulk heterojunction (BHJ) OPV devices. In an OFET, for example, the active semiconductor channel between the drain and source may comprise the layer of the invention. As another example, in an OLED device, the charge (hole or electron) injection or transport layer may comprise the layer of the invention.

For use in OPV or OPD devices the polymer according to the present invention is preferably used in a composition that comprises or contains, preferably consists of, one or more p-type semiconductors and one or more n-type semiconductors.

In a preferred embodiment at least one of the p-type semiconductors in the composition is a compound according to the present invention which is preferably a conjugated polymer. In this preferred embodiment the n-type semiconductor is preferably a fullerene or substituted fullerene.

In another preferred embodiment at least one of the n-type semiconductors in the composition is a compound according to the present invention which is preferably a small molecule, very preferably a compound of formula VI. In this preferred embodiment the p-type semiconductor is preferably a conjugated polymer.

In another preferred embodiment the OPV or OPD device comprises a composition comprising a compound according to the present invention as first n-type semiconductor, and further comprising an p-type semiconductor like a conjugated polymer, and a second n-type semiconductor, which is preferably a fullerene or substituted fullerene.

The n-type semiconductor or second n-type semiconductor in the composition of the aforementioned embodiments is for example an inorganic material such as zinc oxide (ZnOₓ), zinc tin oxide (ZTO), titanium oxide (TiOₓ), molybdenum oxide (MoOₓ), nickel oxide (NiOₓ), or cadmium selenide (CdSe), or an organic material such as graphene or a fullerene, a conjugated polymer or a fullerene or substituted fullerene.

The fullerene is for example an indene-C₆₀-fullerene bisaduct like ICBA, or a (6,6)-phenyl-butyric acid methyl ester derivatized methano C₆₀ fullerene, also known as “PCBM-C₆₀” or “C₆₀PCBM”, as disclosed for example in G. Yu, J. Gao, J. C. Hummelen, F. Wudl, A. J. Heeger, Science 1995, Vol. 270, p. 1789 ff and having the structure shown below, or structural analogous compounds with e.g. a C₆₁ fullerene group, a C₇₀ fullerene group, or a C₇₁ fullerene group, or an organic polymer (see for example Coakley, K. M. and McGehee, M. D. Chem. Mater. 2004, 16, 4533).

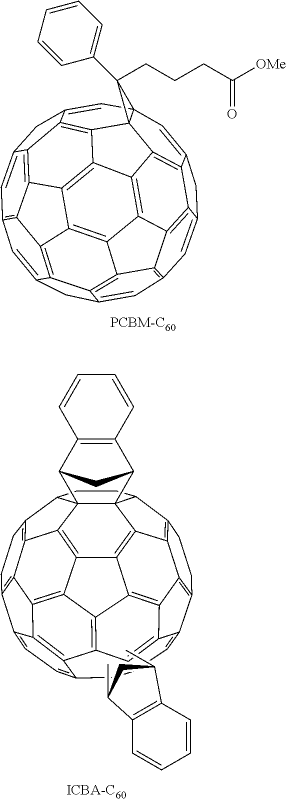

Preferably the fullerene is PCBM-C60, PCBM-C70, bis-PCBM-C60, bis-PCBM-C70, ICMA-c60 (1′,4′-dihydro-naphtho[2′,3′:1,2][5,6]fullerene-C60), ICBA, oQDM-C60 (1′,4′-dihydro-naphtho[2′,3′:1,9][5,6]fullerene-C60-Ih), or bis-oQDM-C60.

Further preferably the n-type semiconductor or second n-type semiconductor in the composition of the aforementioned embodiments is a fullerene or substituted fullerene of formula XII,

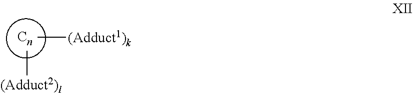

Cₙ denotes a fullerene composed of n carbon atoms, optionally with one or more atoms trapped inside,
Adduct¹ is a primary adduct appended to the fullerene Cₙ with any connectivity,
Adduct² is a secondary adduct, or a combination of secondary adducts, appended to the fullerene Cₙ with any connectivity,
k is an integer ≥1,
and
l is 0, an integer ≥1, or a non-integer >0.

In the formula XII and its subformulae, k preferably denotes 1, 2, 3 or, 4, very preferably 1 or 2.

The fullerene Cₙ in formula XII and its subformulae may be composed of any number n of carbon atoms Preferably, in the compounds of formula XII and its subformulae the number of carbon atoms n of which the fullerene Cₙ is composed is 60, 70, 76, 78, 82, 84, 90, 94 or 96, very preferably 60 or 70.

The fullerene Cₙ in formula XII and its subformulae is preferably selected from carbon based fullerenes, endohedral fullerenes, or mixtures thereof, very preferably from carbon based fullerenes.

Suitable and preferred carbon based fullerenes include, without limitation, (C_{60-Ih})[5,6]fullerene, (C_{70-D5h})[5,6]fullerene, (C_{76-D2*})[5,6]fullerene, (C_{84-D2*})[5,6]fullerene, (C_{84-D2d})[5,6]fullerene, or a mixture of two or more of the aforementioned carbon based fullerenes.

The endohedral fullerenes are preferably metallofullerenes. Suitable and preferred metallofullerenes include, without limitation, La@C₆₀, La@C₈₂, Y@C₈₂, Sc₃N@C₈₀, Y₃N@C₈₀, Sc₃C₂@C₈₀ or a mixture of two or more of the aforementioned metallofullerenes.

Preferably the fullerene Cₙ is substituted at a [6,6] and/or [5,6] bond, preferably substituted on at least one [6,6] bond.

Primary and secondary adduct, named “Adduct” in formula XII and its subformulae, is preferably selected from the following formulae

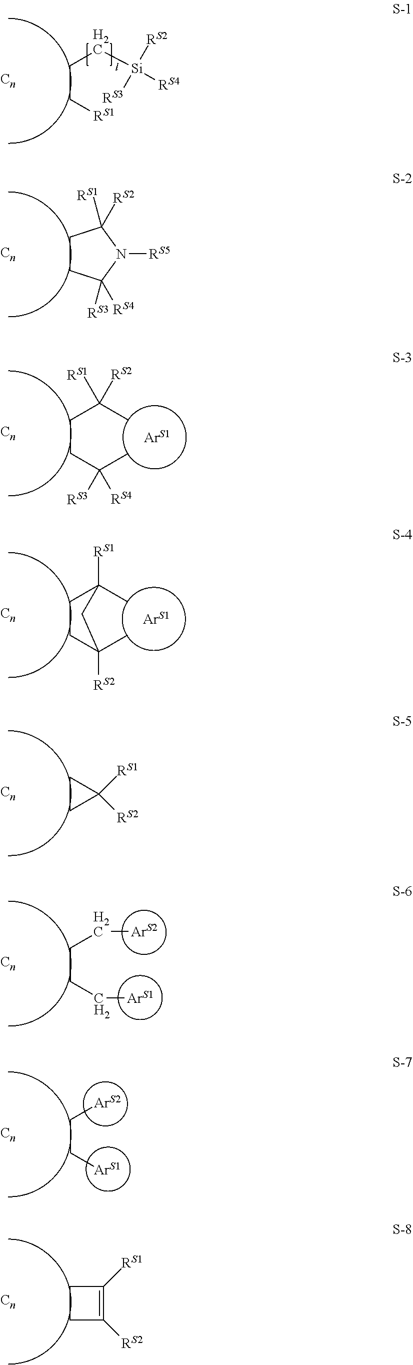

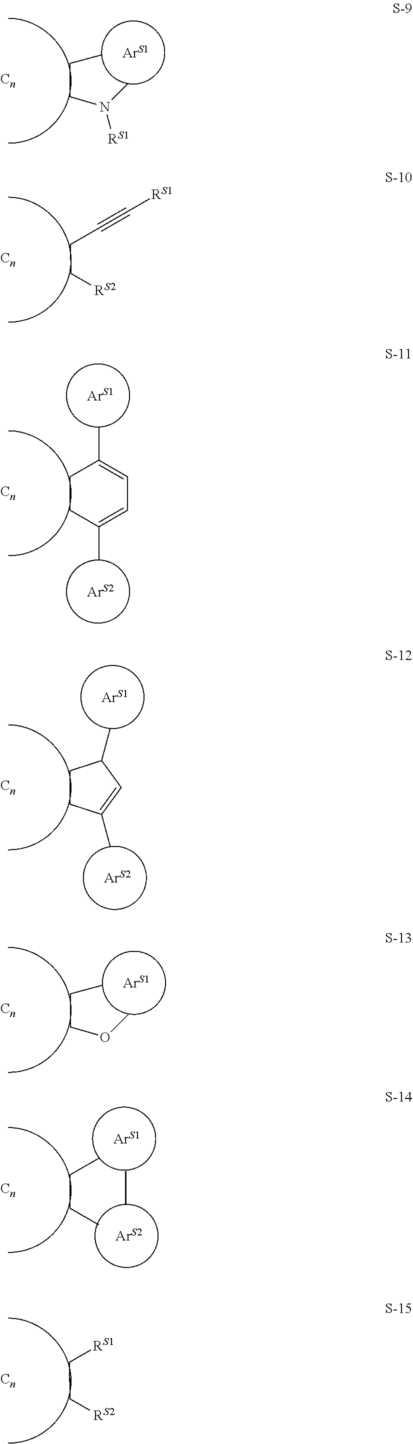

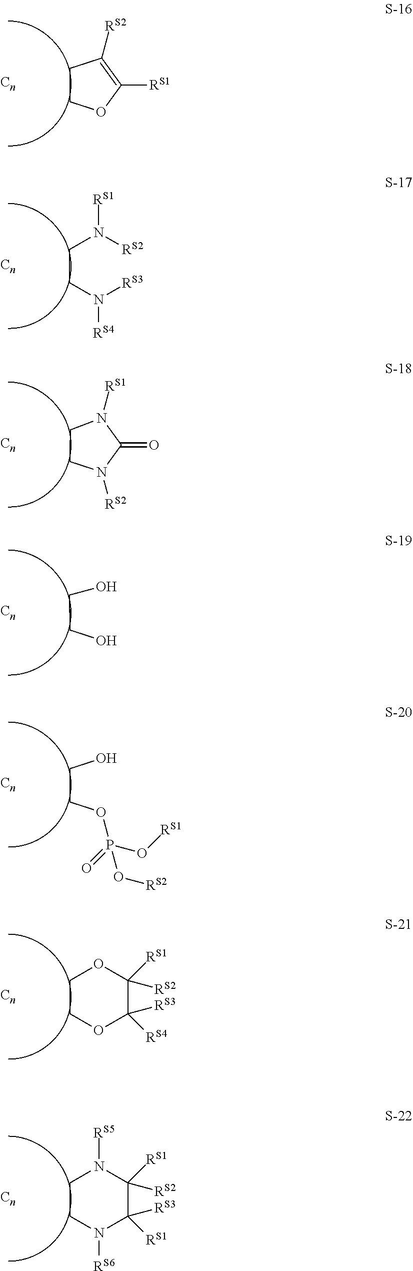

wherein Cₙ is as defined in formula XII,
Ar^{S1}, Ar^{S2} denote, independently of each other, an arylene or heteroarylene group with 5 to 20, preferably 5 to 15, ring atoms, which is mono- or polycyclic, and which is optionally substituted by one or more identical or different substituents having one of the meanings of L as defined above and below, and
R^{S1}, R^{S2}, R^{S3}, R^{S4}, R^{S5} and R^{S6} independently of each other denote H, CN or have one of the meanings of L as defined above and below.

Preferred compounds of formula XII are selected from the following subformulae:

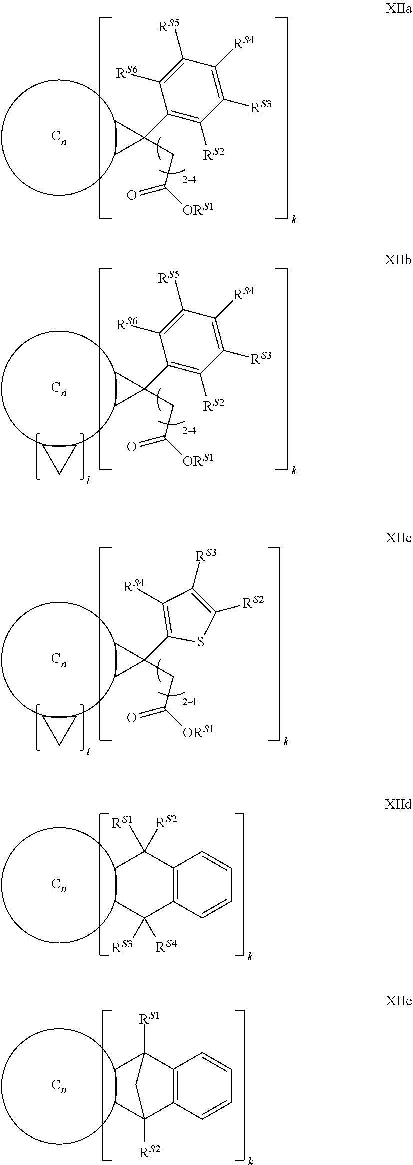

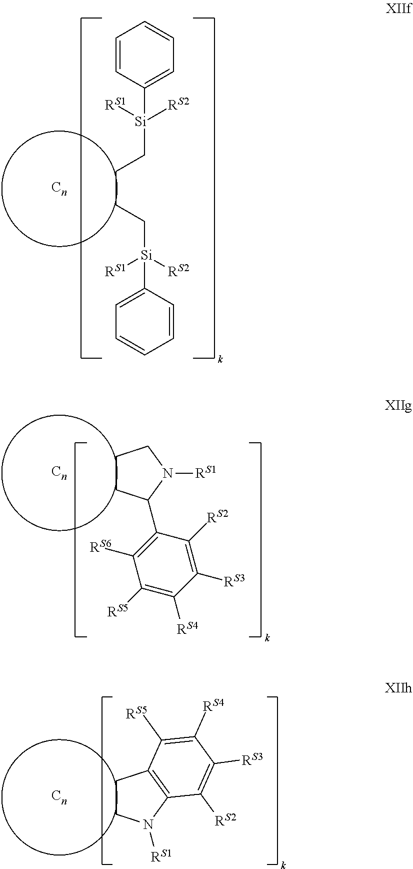

wherein Cₙ, k and l are as defined in formula XII, and

R^{S1}, R^{S2}, R^{S3}, R^{S4} R^{S5} and R^{S6} independently of each other denote H or have one of the meanings of L as defined above and below.

Further preferably the n-type semiconductor or second n-type semiconductor in the composition of the aforementioned embodiments is selected from graphene, metal oxides, like for example, ZnOx, TiOx, ZTO, MoOx, NiOx, quantum dots, like for example, CdSe or CdS, or conjugated polymers, like for example a polynaphthalenediimide or polyperylenediimide as described, for example, in WO2013142841 A1.

The OPV or OPD device according to the present invention preferably comprises a first transparent or semi-transparent electrode on a transparent or semi-transparent substrate on one side of the active layer, and a second metallic or semi-transparent electrode on the other side of the active layer.

Preferably, the photoactive layer in an OPV or OPD device according to the present invention is further blended with additional organic and inorganic compounds to enhance the device properties. For example, metal particles such as Au or Ag nanoparticules or Au or Ag nanoprism for enhancements in light harvesting due to near-field effects (i.e. plasmonic effect) as described, for example in Adv. Mater. 2013, 25 (17), 2385-2396 and Adv. Ener. Mater. 10.1002/aenm.201400206, a molecular dopant such as 2,3,5,6-tetrafluoro-7,7,8,8-tetracyanoquinodimethane for enhancement in photoconductivity as described, for example in Adv. Mater. 2013, 25(48), 7038-7044, or a stabilising agent consisting of a UV absorption agent and/or anti-radical agent and/or antioxidant agent such as 2-hydroxybenzophenone, 2-hydroxyphenylbenzotriazole, oxalic acid anilides, hydroxyphenyl triazines, merocyanines, hindered phenol, N-aryl-thiomorpholine, N-aryl-thiomorpholine-1-oxide, N-aryl-thiomorpholine-1,1-dioxide, N-aryl-thiazolidine, N-aryl-thiazolidine-1-oxide, N-aryl-thiazolidine-1,1-dioxide and 1,4-diazabicyclo[2.2.2]octane as described, for example, in WO2012095796 A1 and in WO2013021971 A1.

The device preferably may further comprise a UV to visible photo-conversion layer such as described, for example, in J. Mater. Chem. 2011, 21, 12331 or a NIR to visible or IR to NIR photo-conversion layer such as described, for example, in J. Appl. Phys. 2013, 113, 124509.

Further preferably the OPV or OPD device comprises, between the active layer and the first or second electrode, one or more additional buffer layers acting as hole transporting layer and/or electron blocking layer, which comprise a material such as metal oxides, like for example, ZTO, MoOₓ, NiOₓ, a doped conjugated polymer, like for example PEDOT:PSS and polypyrrole-polystyrene sulfonate (PPy:PSS), a conjugated polymer, like for example polytriarylamine (PTAA), an organic compound, like for example substituted triaryl amine derivatives such as N,N′-diphenyl-N,N′-bis(1-naphthyl)(1,1′-biphenyl)-4,4′diamine (NPB), N,N′-diphenyl-N,N′-(3-methylphenyl)-1,1′-biphenyl-4,4′-diamine (TPD), graphene based materials, like for example, graphene oxide and graphene quantum dots or alternatively as hole blocking layer and/or electron transporting layer, which comprise a material such as metal oxide, like for example, ZnOₓ, TiOₓ, AZO (aluminium doped zinc oxide), a salt, like for example LiF, NaF, CsF, a conjugated polymer electrolyte, like for example poly[3-(6-trimethylammoniumhexyl)thiophene], poly(9,9-bis(2-ethylhexyl)-fluorene]-b-poly[3-(6-trimethylammoniumhexyl)thiophene], or poly[(9,9-bis(3′-(N,N-dimethylamino)propyl)-2,7-fluorene)-alt-2,7-(9,9-dioctylfluorene)], a polymer, like for example poly(ethyleneimine) or crosslinked N-containing compound derivatives or an organic compound, like for example tris(8-quinolinolato)-aluminium(III) (Alq₃), phenanthroline derivative or C₆₀ or C₇₀ based fullerenes, like for example, as described in Adv. Energy Mater. 2012, 2, 82-86.

In a composition comprising a small molecule compound according to the present invention and further comprising a polymer, the ratio polymer:small molecule compound is preferably from 5:1 to 1:5 by weight, more preferably from 1:1 to 1:3 by weight, most preferably 1:1 to 1:2 by weight.

In a composition comprising a polymer compound according to the present invention and further comprising a fullerene or modified fullerene, the ratio polymer:fullerene is preferably from 5:1 to 1:5 by weight, more preferably from 2:1 to 1:3 by weight, most preferably 1:1 to 1:2 by weight.

The composition according to the present invention may also comprise polymeric binder, preferably from 5 to 95% by weight. Examples of binder include polystyrene (PS), polypropylene (PP), polydimethylsilane (PDMS), and polymethylmethacrylate (PMMA).

To produce thin layers in BHJ OPV devices the compounds, compositions and formulations of the present invention may be deposited by any suitable method. Liquid coating of devices is more desirable than vacuum deposition techniques. Solution deposition methods are especially preferred. The formulations of the present invention enable the use of a number of liquid coating techniques. Preferred deposition techniques include, without limitation, dip coating, spin coating, ink jet printing, nozzle printing, letter-press printing, screen printing, gravure printing, doctor blade coating, roller printing, reverse-roller printing, offset lithography printing, dry offset lithography printing, flexographic printing, web printing, spray coating, curtain coating, brush coating, slot dye coating or pad printing. For the fabrication of OPV devices and modules area printing method compatible with flexible substrates are preferred, for example slot dye coating, spray coating and the like.

In the preparation of a formulation according to the present invention, suitable solvents are preferably selected to ensure full dissolution of both the p-type and n-type component, and take into account the boundary conditions (for example rheological properties) introduced by the chosen printing method.

Organic solvent are generally used for this purpose. Typical solvents can be aromatic solvents, halogenated solvents or chlorinated solvents, including chlorinated aromatic solvents. Examples include, but are not limited to chlorobenzene, 1,2-dichlorobenzene, chloroform, 1,2-dichloroethane, dichloromethane, carbon tetrachloride, toluene, cyclohexanone, ethylacetate, tetrahydrofuran, anisole, 2,4-dimethylanisole, 1-methylnaphthalene, morpholine, toluene, o-xylene, m-xylene, p-xylene, 1,4-dioxane, acetone, methylethylketone, 1,2-dichloroethane, 1,1,1-trichloroethane, 1,1,2,2-tetrachloroethane, ethyl acetate, n-butyl acetate, N,N-dimethylformamide, dimethylacetamide, dimethylsulfoxide, 1,5-dimethyltetraline, propiophenone, acetophenone, tetraline, 2-methylthiophene, 3-methylthiophene, decaline, indane, methyl benzoate, ethyl benzoate, mesitylene and combinations thereof.

The OPV device can for example be of any type known from the literature (see e.g. Waldauf et al., Appl. Phys. Lett., 2006, 89, 233517).

A first preferred OPV device according to the invention comprises the following layers (in the sequence from bottom to top):
optionally a substrate,
a high work function electrode, preferably comprising a metal oxide, like for example ITO and FTO, serving as anode,
an optional conducting polymer layer or hole transport layer, preferably comprising an organic polymer or polymer blend, for example PEDOT:PSS (poly(3,4-ethylenedioxythiophene):poly(styrene-sulfonate), substituted triaryl amine derivatives, for example, TBD (N,N′-dyphenyl-N—N′-bis(3-methylphenyl)-1,1′biphenyl-4,4′-diamine) or NBD (N,N′-dyphenyl-N—N′-bis(1-napthylphenyl)-1,1′biphenyl-4,4′-diamine),
a layer, also referred to as “photoactive layer”, comprising a p-type and an n-type organic semiconductor, which can exist for example as a p-type/n-type bilayer or as distinct p-type and n-type layers, or as blend or p-type and n-type semiconductor, forming a BHJ,
optionally a layer having electron transport properties, for example comprising LiF, TiOₓ, ZnOₓ, PFN, a poly(ethyleneimine) or crosslinked nitrogen containing compound derivatives or a phenanthroline derivatives
a low work function electrode, preferably comprising a metal like for example aluminum, serving as cathode,
wherein at least one of the electrodes, preferably the anode, is transparent to visible and/or NIR light, and
wherein the p-type or n-type semiconductor is a compound according to the present invention.

A second preferred OPV device according to the invention is an inverted OPV device and comprises the following layers (in the sequence from bottom to top):
optionally a substrate,
a high work function metal or metal oxide electrode, comprising for example ITO and FTO, serving as cathode,
a layer having hole blocking properties, preferably comprising a metal oxide like TiOₓ or ZnOₓ, or comprising an organic compound such as polymer like poly(ethyleneimine) or crosslinked nitrogen containing compound derivatives or phenanthroline derivatives,
a photoactive layer comprising a p-type and an n-type organic semiconductor, situated between the electrodes, which can exist for example as a p-type/n-type bilayer or as distinct p-type and n-type layers, or as blend or p-type and n-type semiconductor, forming a BHJ,
an optional conducting polymer layer or hole transport layer, preferably comprising an organic polymer or polymer blend, for example of PEDOT:PSS, nafion or substituted triaryl amine derivatives, for example TBD or NBD,
an electrode comprising a high work function metal like for example silver, serving as anode,
wherein at least one of the electrodes, preferably the cathode, is transparent to visible and/or NIR light, and
wherein the p-type or n-type semiconductor is a compound according to the present invention.

In the OPV devices of the present invention the p-type and n-type semiconductor materials are preferably selected from the materials, like the compound/polymer or compound/polymer/fullerene systems as described above.

When the active layer is deposited on the substrate, it forms a BHJ that phase separates at nanoscale level. For discussion on nanoscale phase separation see Dennler et al, Proceedings of the IEEE, 2005, 93 (8), 1429 or Hoppe et al, Adv. Func. Mater, 2004, 14(10), 1005. An optional annealing step may be then necessary to optimize blend morpohology and consequently OPV device performance.

Another method to optimize device performance is to prepare formulations for the fabrication of OPV(BHJ) devices that may include high boiling point additives to promote phase separation in the right way. 1,8-Octanedithiol, 1,8-diiodooctane, nitrobenzene, 1-chloronaphthalene, N,N-dimethylformamide, dimethylacetamide, dimethylsulfoxide and other additives have been used to obtain high-efficiency solar cells. Examples are disclosed in J. Peet, et al, Nat. Mater., 2007, 6, 497 or Fréchet et al. J. Am. Chem. Soc., 2010, 132, 7595-7597.

Another preferred embodiment of the present invention relates to the use of a compound or composition according to the present invention as dye, hole transport layer, hole blocking layer, electron transport layer and/or electron blocking layer in a DSSC or a perovskite-based solar cells, and to a DSSC or perovskite-based solar cells comprising a compound composition or polymer blend according to the present invention.

DSSCs and perovskite-based DSSCs can be manufactured as described in the literature, for example in Chem. Rev. 2010, 110, 6595-6663, Angew. Chem. Int. Ed. 2014, 53, 2-15 or in WO2013171520A1

The compounds and compositions of the present invention are also suitable for use in the semiconducting channel of an OFET. Accordingly, the invention also provides an OFET comprising a gate electrode, an insulating (or gate insulator) layer, a source electrode, a drain electrode and an organic semiconducting channel connecting the source and drain electrodes, wherein the organic semiconducting channel comprises a compound or composition according to the present invention. Other features of the OFET are well known to those skilled in the art.

OFETs where an OSC material is arranged as a thin film between a gate dielectric and a drain and a source electrode, are generally known, and are described for example in U.S. Pat. Nos. 5,892,244, 5,998,804, 6,723,394 and in the references cited in the background section. Due to the advantages, like low cost production using the solubility properties of the compounds according to the invention and thus the processibility of large surfaces, preferred applications of these FETs are such as integrated circuitry, TFT displays and security applications.

The gate, source and drain electrodes and the insulating and semiconducting layer in the OFET device may be arranged in any sequence, provided that the source and drain electrode are separated from the gate electrode by the insulating layer, the gate electrode and the semiconductor layer both contact the insulating layer, and the source electrode and the drain electrode both contact the semiconducting layer.

An OFET device according to the present invention preferably comprises:
a source electrode,
a drain electrode,
a gate electrode,
a semiconducting layer,
one or more gate insulator layers,
optionally a substrate.
   wherein the semiconductor layer comprises a compound or composition according to the present invention.

The OFET device can be a top gate device or a bottom gate device.

Suitable structures and manufacturing methods of an OFET device are known to the skilled in the art and are described in the literature, for example in US 2007/0102696 A1.

The gate insulator layer preferably comprises a fluoropolymer, like e.g. the commercially available Cytop 809M® or Cytop 107M® (from Asahi Glass).

Preferably the gate insulator layer is deposited, e.g. by spin-coating, doctor blading, wire bar coating, spray or dip coating or other known methods, from a formulation comprising an insulator material and one or more solvents with one or more fluoro atoms (fluorosolvents), preferably a perfluorosolvent. A suitable perfluorosolvent is e.g. FC75® (available from Acros, catalogue number 12380). Other suitable fluoropolymers and fluorosolvents are known in prior art, like for example the perfluoropolymers Teflon AF® 1600 or 2400 (from DuPont) or Fluoropel® (from Cytonix) or the perfluorosolvent FC 43® (Acros, No. 12377). Especially preferred are organic dielectric materials having a low permittivity (or dielectric constant) from 1.0 to 5.0, very preferably from 1.8 to 4.0 (“low k materials”), as disclosed for example in US 2007/0102696 A1 or U.S. Pat. No. 7,095,044.

In security applications, OFETs and other devices with semiconducting materials according to the present invention, like transistors or diodes, can be used for RFID tags or security markings to authenticate and prevent counterfeiting of documents of value like banknotes, credit cards or ID cards, national ID documents, licenses or any product with monetary value, like stamps, tickets, shares, cheques etc.

Alternatively, the compounds and compositions according to the invention can be used in OLEDs, e.g. as the active display material in a flat panel display applications, or as backlight of a flat panel display like e.g. a liquid crystal display. Common OLEDs are realized using multilayer structures. An emission layer is generally sandwiched between one or more electron-transport and/or hole-transport layers. By applying an electric voltage electrons and holes as charge carriers move towards the emissive layer where their recombination leads to the excitation and hence luminescence of the lumophor units contained in the emission layer.

The compounds and compositions according to the invention can be employed in one or more of a buffer layer, electron or hole transport layer, electron or hole blocking layer and emissive layer, corresponding to their electrical and/or optical properties. Furthermore their use within the emissive layer is especially advantageous, if the compounds according to the invention show electroluminescent properties themselves or comprise electroluminescent groups or compounds. The selection, characterization as well as the processing of suitable monomeric, oligomeric and polymeric compounds or materials for the use in OLEDs is generally known by a person skilled in the art, see, e.g., Müller et al, Synth. Metals, 2000, 111-112, 31-34, Alcala, J. Appl. Phys., 2000, 88, 7124-7128 and the literature cited therein.

According to another use, the compounds and compositions according to the present invention, especially those showing photoluminescent properties, may be employed as materials of light sources, e.g. in display devices, as described in EP 0 889 350 A1 or by C. Weder et al., Science, 1998, 279, 835-837.

A further aspect of the invention relates to both the oxidised and reduced form of a compound according to the present invention. Either loss or gain of electrons results in formation of a highly delocalised ionic form, which is of high conductivity. This can occur on exposure to common dopants. Suitable dopants and methods of doping are known to those skilled in the art, e.g. from EP 0 528 662, U.S. Pat. No. 5,198,153 or WO 96/21659.

The doping process typically implies treatment of the semiconductor material with an oxidating or reducing agent in a redox reaction to form delocalised ionic centres in the material, with the corresponding counterions derived from the applied dopants. Suitable doping methods comprise for example exposure to a doping vapor in the atmospheric pressure or at a reduced pressure, electrochemical doping in a solution containing a dopant, bringing a dopant into contact with the semiconductor material to be thermally diffused, and ion-implantantion of the dopant into the semiconductor material.

When electrons are used as carriers, suitable dopants are for example halogens (e.g., I₂, Cl₂, Br₂, ICl, ICl₃, IBr and IF), Lewis acids (e.g., PF₅, AsF₅, SbF₅, BF₃, BCl₃, SbCl₅, BBr₃ and SO₃), protonic acids, organic acids, or amino acids (e.g., HF, HCl, HNO₃, H₂SO₄, HClO₄, FSO₃H and ClSO₃H), transition metal compounds (e.g., FeCl₃, FeOCl, Fe(ClO₄)₃, Fe(4-CH₃C₆H₄SO₃)₃, TiCl₄, ZrCl₄, HfCl₄, NbF₅, NbCl₅, TaCl₅, MoF₅, MoCl₅, WF₅, WCl₆, UF₆ and LnCl₃ (wherein Ln is a lanthanoid), anions (e.g., Cl⁻, Br⁻, I⁻, I₃⁻, HSO₄⁻, SO₄²⁻, NO₃⁻, ClO₄⁻, BF₄⁻, PF₆⁻, AsF₆⁻, SbF₆⁻, FeCl₄⁻, Fe(CN)₆³⁻, and anions of various sulfonic acids, such as aryl-SO₃). When holes are used as carriers, examples of dopants are cations (e.g., H⁺, Li⁺, Na⁺, K⁺, Rb⁺ and Cs⁺), alkali metals (e.g., Li, Na, K, Rb, and Cs), alkaline-earth metals (e.g., Ca, Sr, and Ba), O₂, XeOF₄, (NO₂⁺) (SbF₆⁻), (NO₂⁺) (SbCl₆⁻), (NO₂⁺) (BF₄⁻), AgClO₄, H₂IrCl₆, La(NO₃)₃.6H₂O, FSO₂OOSO₂F, Eu, acetylcholine, R₄N⁺, (R is an alkyl group), R₄P⁺ (R is an alkyl group), R₆As⁺ (R is an alkyl group), and R₃S⁺ (R is an alkyl group).

The conducting form of a compound according to the present invention can be used as an organic “metal” in applications including, but not limited to, charge injection layers and ITO planarising layers in OLED applications, films for flat panel displays and touch screens, antistatic films, printed conductive substrates, patterns or tracts in electronic applications such as printed circuit boards and condensers.

The compounds and compositions according to the present invention may also be suitable for use in organic plasmon-emitting diodes (OPEDs), as described for example in Koller et al., Nat. Photonics, 2008, 2, 684.

According to another use, the compounds according to the present invention can be used alone or together with other materials in or as alignment layers in LCD or OLED devices, as described for example in US 2003/0021913. The use of charge transport compounds according to the present invention can increase the electrical conductivity of the alignment layer. When used in an LCD, this increased electrical conductivity can reduce adverse residual dc effects in the switchable LCD cell and suppress image sticking or, for example in ferroelectric LCDs, reduce the residual charge produced by the switching of the spontaneous polarisation charge of the ferroelectric LCs. When used in an OLED device comprising a light emitting material provided onto the alignment layer, this increased electrical conductivity can enhance the electroluminescence of the light emitting material. The compounds according to the present invention having mesogenic or liquid crystalline properties can form oriented anisotropic films as described above, which are especially useful as alignment layers to induce or enhance alignment in a liquid crystal medium provided onto said anisotropic film. The polymers according to the present invention may also be combined with photoisomerisable compounds and/or chromophores for use in or as photoalignment layers, as described in US 2003/0021913 A1.

According to another use the compounds and compositions according to the present invention, especially their water-soluble derivatives (for example with polar or ionic side groups) or ionically doped forms, can be employed as chemical sensors or materials for detecting and discriminating DNA sequences. Such uses are described for example in L. Chen, D. W. McBranch, H. Wang, R. Helgeson, F. Wudl and D. G. Whitten, Proc. Natl. Acad. Sci. U.S.A., 1999, 96, 12287; D. Wang, X. Gong, P. S. Heeger, F. Rininsland, G. C. Bazan and A. J. Heeger, Proc. Natl. Acad. Sci. U.S.A., 2002, 99, 49; N. DiCesare, M. R. Pinot, K. S. Schanze and J. R. Lakowicz, Langmuir, 2002, 18, 7785; D. T. McQuade, A. E. Pullen, T. M. Swager, Chem. Rev., 2000, 100, 2537.

Unless the context clearly indicates otherwise, as used herein plural forms of the terms herein are to be construed as including the singular form and vice versa.

Throughout the description and claims of this specification, the words “comprise” and “contain” and variations of the words, for example “comprising” and “comprises”, mean “including but not limited to”, and are not intended to (and do not) exclude other components.

It will be appreciated that variations to the foregoing embodiments of the invention can be made while still falling within the scope of the invention. Each feature disclosed in this specification, unless stated otherwise, may be replaced by alternative features serving the same, equivalent or similar purpose. Thus, unless stated otherwise, each feature disclosed is one example only of a generic series of equivalent or similar features.

All of the features disclosed in this specification may be combined in any combination, except combinations where at least some of such features and/or steps are mutually exclusive. In particular, the preferred features of the invention are applicable to all aspects of the invention and may be used in any combination. Likewise, features described in non-essential combinations may be used separately (not in combination).

Above and below, unless stated otherwise percentages are percent by weight and temperatures are given in degrees Celsius. Values of the dielectric constant E (“permittivity”) refer to values taken at 20° C. and 1,000 Hz.

The invention will now be described in more detail by reference to the following examples, which are illustrative only and do not limit the scope of the invention.

### Example 1

### 3,6-Dibromo-4,5-dichlorophenylene-1,2-diamine (1.1)

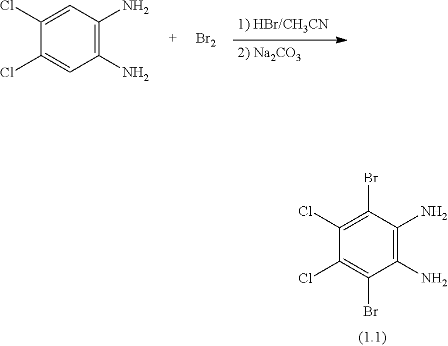 [SMILES: Nc1cc(Cl)c(Cl)cc1N ; "Br2" ; Nc1c(N)c(Br)c(Cl)c(Cl)c1Br]

To a solution of 4,5-dichlorophenylene-1,2-diamine (9.0 g, 50 mmol) in acetonitrile (250 cm³) was added 48% hydrobromic acid (45.0 cm³, 398 mmol). The suspension was heated and stirred at reflux for 1 hour. Bromine (10.0 cm³, 195.2 mmol) was added dropwise to afford an orange suspension. The heating was stopped and the suspension was stirred at 20° C. for 2 hours. The precipitate was suction filtered off and washed with acetonitrile until the wash was colourless. The solid on the filter was air-dried and transferred into a conical flask. Water (150 cm³) was added followed by the addition of sodium carbonate in small portions under stirring until the pH of the mixture was ca 8. The solid was collected by suction filtration and washed with water and air-dried on the filter to yield 3,6-dibromo-4,5-dichlorophenylene-1,2-diamine as a pale-tan solid (13.7 g, 82%). m/e (EI): 334, 100%. ¹³C-NMR (DMSO-d₆, 100.6 MHz): δ 106.3, 118.7, 133.3 ppm.

### 4,7-Dibromo-5,6-dichlorobenzo-2,1,3-thiadiazole (1.2)

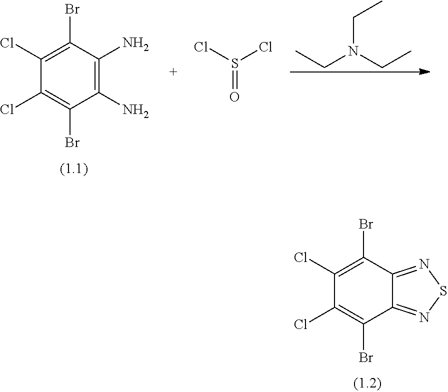

To a mixture of anhydrous dichloromethane (150 cm³) and triethylamine (28.4 cm³, 204.9 mmol) was added 3,6-dibromo-4,5-dichlorophenylene-1,2-diamine (13.72 g, 41.0 mmol). The suspension was stirred at −78° C. Thionyl chloride (5.95 cm³, 82.0 mmol) was added dropwise over 20 minutes. The cooling bath was removed and the suspension was heated at reflux for 2 hours. The solvent was removed in vacuo to yield a solid residue. Methanol (200 cm³) was added and the solid was triturated and then collected by suction filtration. The solid on the filter was washed with methanol until the filtrate was colourless, then air dried, to afford 4,7-bibromo-5,6-dichlorobenzo-2,1,3-thiadiazole as an off-white solid (13.8 g, 93%). m/e (EI): 362 (100%). ¹³C-NMR (CDCl₃, 100.6 MHz): δ ppm 114.9, 136.3, 151.2.

### 5,6-Dichloro-4,7-bis(2-thienyl)-2,1,3-benzothiadiazole (1.3)

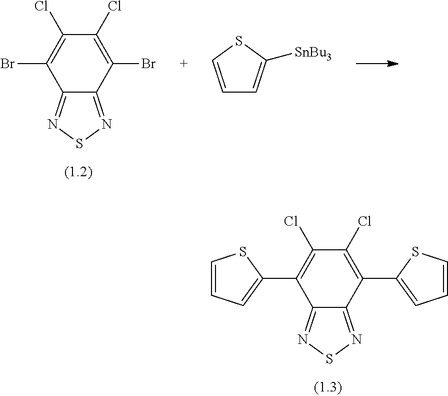

To a solution of 4,7-dibromo-5,6-dichlorobenzo-2,1,3-thiadiazole (9.07 g, 25.0 mmol) in toluene (80 cm³) and N,N-dimethylformamide (20 cm³) were added tributyl(thien-2-yl)stannane (20.0 cm³, 61.1 mmol), tris(dibenzylideneacetone)dipalladium(0) (228.9 mg, 0.250 mmol) and tri(o-tolyl)phosphine (380.5 mg, 1.250 mmol). The mixture was stirred at 100° C. under nitrogen for 1 hour. The solvents were evaporated in vacuo and the solid residue was triturated with methanol (100 cm³). The solid was collected by suction filtration and washed with methanol 3 times. The solid was dissolved in chloroform and the solution was filtered through a silica plug. The filtrate was evaporated in vacuo to afford 5,6-dichloro-4,7-bis(2-thienyl)-2,1,3-benzothiadiazole as red-orange crystals (7.66 g, 83%). m/e (EI): [368, M+] 100%. ¹H NMR (400 MHz, methylene chloride-d2) δ 7.69-7.61 (m, 2H), 7.26 (dd, J=5.1, 3.7 Hz, 1H). ¹³C NMR (101 MHz, dichloromethane-d2) δ 152.71, 134.72, 134.21, 131.30, 128.28, 126.77, 126.30 ppm.

### 4,7-Bis(5-bromo-2-thienyl)-5,6-dichloro-2,1,3-benzothiadiazole (1.4)

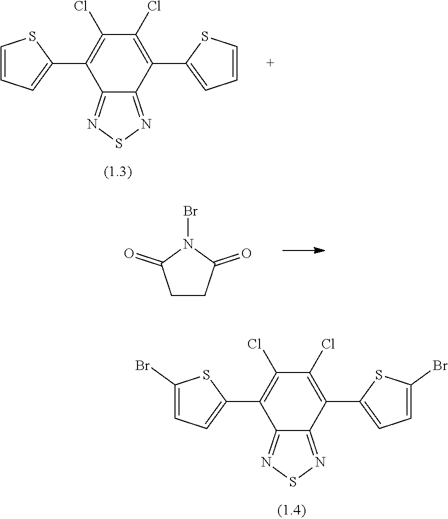 [SMILES: Clc1c(Cl)c(-c2cccs2)c2nsnc2c1-c1cccs1 ; O=C1CCC(=O)N1Br ; Clc1c(Cl)c(-c2ccc(Br)s2)c2nsnc2c1-c1ccc(Br)s1]

To a solution of 5,6-dichloro-4,7-bis(2-thienyl)-2,1,3-benzothiadiazole (3.69 g, 10.0 mmol) in anhydrous tetrahydrofuran (100 cm³) was added N-bromosuccinimide (5.39 g, 30.0 mmol) in one portion. The mixture was stirred at 20° C. for 20 hours, and then at reflux for 30 minutes. The mixture was cooled to 23° C. and methanol (50 cm³) was added. The precipitate was collected by suction filtration, washed with methanol and then air-dried to afford 4,7-bis(5-bromo-2-thienyl)-5,6-dichloro-2,1,3-benzothiadiazole as orange crystals (4.82 g, 91%). m/e (EI): 527 (M⁺) 100%. ¹H NMR (400 MHz, Chloroform-d) δ 7.52 (d, J=4.0 Hz, 1H), 7.21 (d, J=3.9 Hz, 1H).

### 4,7-Bis(5-bromo-2-thienyl)-(2-dicyanomethylene[1,3]dithiolo)[4,5-f]-2,1,3-benzothiadiazole (1)

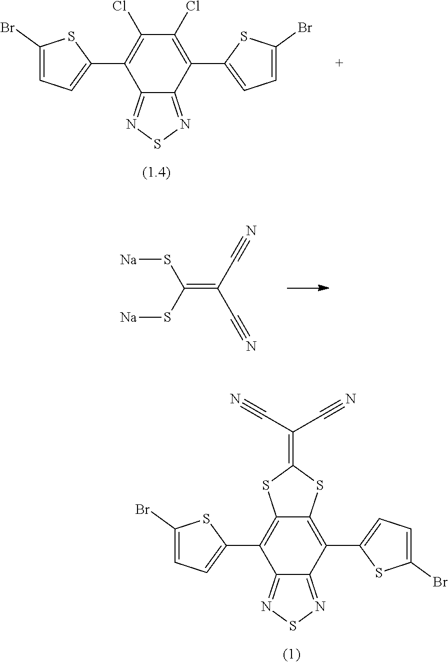

4,7-Bis-(5-bromo-2-thienyl)-5,6-dichloro-2,1,3-benzothiadiazole (0.97 g, 1.84 mmol) and disodium dicyanomethylenemethanedithiolate (0.51 g, 2.76 mmol) were suspended in anhydrous N,N-dimethylformamide (50 cm³). The mixture was heated at 80° C. for 20 hours. The volatile was removed in vacuo. The residue triturated with methanol and the solid collected by suction filtration. The solid was boiled in chloroform (100 cm³) for 1 hour and the mixture was purified by column chromatography (chloroform) followed by recrystallisation (pyridine/ethanol) to afford 4,7-bis(5-bromo-2-thienyl)-(2-dicyanomethylene[1,3]dithiolo)[4,5-f]-2,1,3-benzothiadiazole as a dark-red crystalline solid (0.39 g, 36%). ¹H NMR (400 MHz, Chloroform-d) δ 7.45 (d, J=4.0 Hz, 1H), 7.27 (d, J=4.0 Hz, 1H).

### Example 2

### 5,8-Dibromo-6,7-dichloro-2,3-dimethylquinoxaline (2.1)

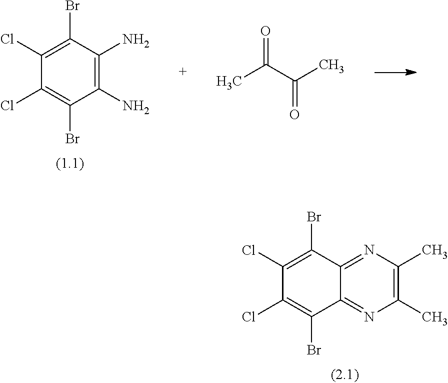

To a mixture of 3,6-dibromo-4,5-dichloro-1,2-phenylenediamine (5.08 g, 15.22 mmol) in acetonitrile (120 cm³) was added butane-2,3-dione (1.65 cm³, 18.21 mmol). The mixture was stirred at reflux for 16 hours and the solvent removed in vacuo. The solid residue was triturated with methanol then collected by suction filtration. The solid was washed with methanol three times then air-dried to give 5,8-dibromo-6,7-dichloro-2,3-dimethylquinoxaline as yellowish crystals (5.20 g, 89%). m/e (EI): 384 (100%). ¹H NMR (400 MHz, Chloroform-d) δ 2.80 (s, 3H). ¹³C NMR (101 MHz, CDCl₃) δ 155.96, 138.23, 134.71, 124.59, 22.94.

### 6,7-Dichloro-2,3-dimethyl-5,8-bis(2-thienyl)quinoxaline (2.2)

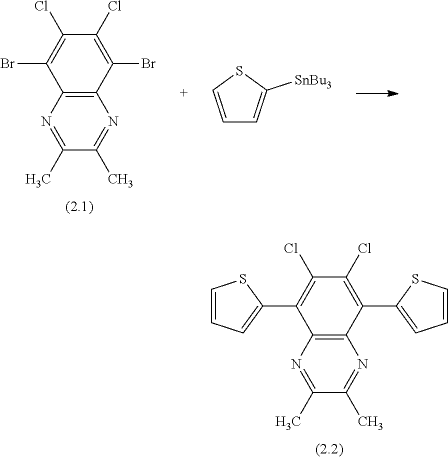

To a solution of 5,8-dibromo-6,7-dichloro-2,3-dimethylquinoxaline (3.85 g, 10.00 mmol) in anhydrous toluene (40 cm³) and N,N-dimethylformamide (10 cm³) were added tributylthien-2-ylstannane (8.0 cm³, 24.0 mmol), tris(dibenzylideneacetone)dipalladium(0) (91.6 mg, 0.100 mmol) and tri(o-tolyl)phosphine (152.2 mg, 0.500 mmol). The mixture was stirred at 100° C. under nitrogen for 1 hour. The solvents were removed in vacuo and the solid residue triturated with methanol (50 cm³). The solid collected by suction filtration and washed with methanol. The crude product was recrystallised (chloroform/ethanol) to give 6,7-dichloro-2,3-dimethyl-5,8-bis(2-thienyl)quinoxaline as yellow crystals (3.52 g, 90%). m/e (EI): 390 (100%). ¹H NMR (400 MHz, Chloroform-d) δ 7.60 (dd, J=5.1, 1.2 Hz, 1H), 7.33 (dd, J=3.5, 1.2 Hz, 1H), 7.23 (dd, J=5.1, 3.5 Hz, 1H), 2.63 (s, 3H). ¹³C NMR (101 MHz, CDCl₃) δ 153.95, 139.08, 135.52, 133.96, 133.09, 130.11, 127.49, 126.32, 23.16.

### Example 3

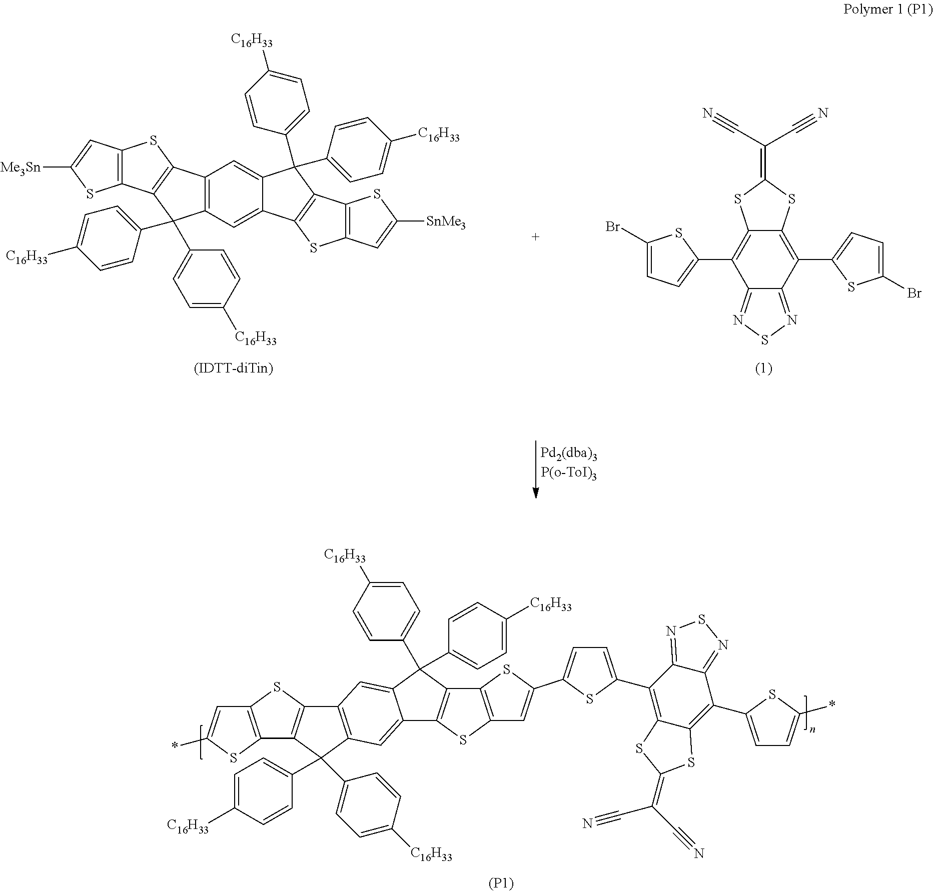

A flask was charged with 4,7-bis(5-bromo-2-thienyl)-(2-dicyanomethylene[1,3]dithiolo)[4,5-f]-2,1,3-benzothiadiazole (1) (178.9 mg, 0.300 mmol), 1,1′-[6,6,12,12-tetrakis(4-hexadecylphenyl)-6,12-dihydrodithieno[2,3-d:2′,3′-d′]-s-indaceno[1,2-b:5,6-b′]dithiophene-2,8-diyl]bis(trimethylstannane) (IDTT-diTin) (571.9 mg, 0.300 mmol), anhydrous toluene (9.0 cm³) and anhydrous N,N-dimethylformamide (1.0 cm³). The mixture was degassed by bubbling nitrogen for 30 minutes and tris(dibenzylideneacetone)dipalladium(0) (10.6 mg, 0.015 mmol) and tri-o-tolylphosphine (41.1 mg, 0.135 mmol) were added. The mixture was degassed for an additional 10 minutes. The mixture was then vigorously stirred at 120° C. under nitrogen for 10 minutes. Tributylphenylstannane (0.50 cm³, 1.53 mmol) was added and the mixture was stirred for another 50 minutes at 120° C. Bromobenzene (2.0 cm³, 18.7 mmol) was added and the mixture was stirred under the same conditions for 1 hour. The solution was diluted with toluene (5 cm³) then precipitated into stirred acetone (100 cm³) and the mixture was stirred at 20° C. for 30 minutes. The solid was collected by suction filtration, washed with acetone to afford polymer 1 (0.45 g, 75%) as a dark-green solid. GPC (chlorobenzene, 50° C.): Mₙ=59,000 Kg/mol, Mw=270,000 kg/mol, PDI=4.6.

### Example 4

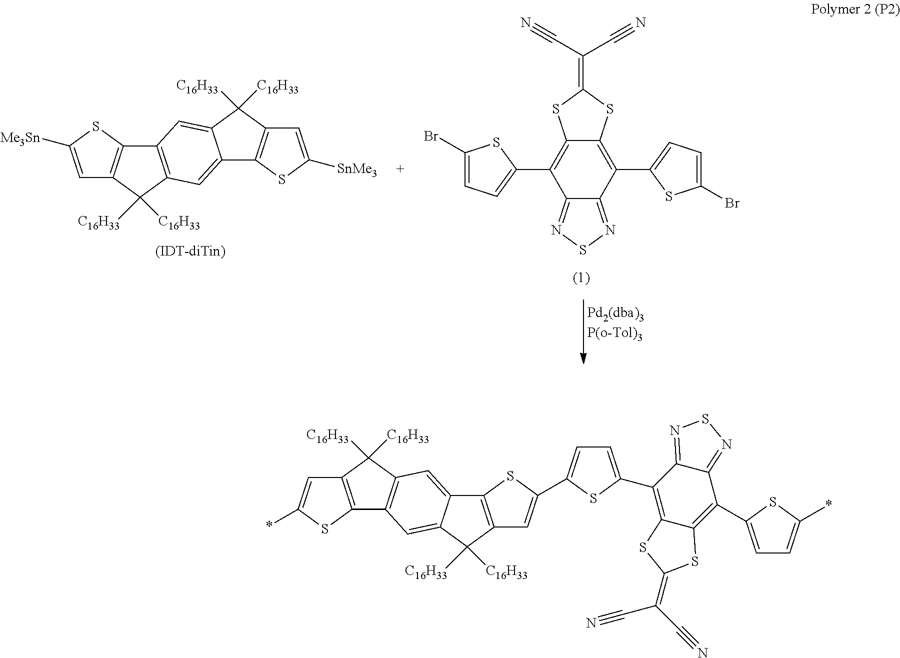

A round-bottom flask was charged with 4,7-bis(5-bromo-2-thienyl)-(2-dicyanomethylene[1,3]dithiolo)[4,5-f]-2,1,3-benzothiadiazole (1) (178.9 mg; 0.300 mmol), IDT-ditin (446.9 mg, 0.300 mmol), anhydrous toluene, (9.0 cm³) and N,N-dimethylformamide (1.0 cm³). The mixture was degassed by bubbling nitrogen for 0.5 hours. Tris(dibenzylideneacetone)dipalladium(0) (10.6 mg, 0.015 mmol) and tri(o-tolyl)phosphine (41.1 mg, 0.135 mmol) were added and the mixture was degassed for an additional 10 minutes. The mixture was stirred at 130° C. (external) under nitrogen for 4 hours. Tributylphenylstannane (0.50 cm³, 1.53 mmol) was added and the mixture was stirred at the same temperature for 30 minutes. Bromobenzene (2.0 cm³, 18.7 mmol) was added and the mixture was stirred under the same conditions for 1 hour. The mixture was precipitated with methanol while it was still hot. The solid was collected by suction filtration and washed with methanol and acetone. The polymer solid was further purified by Soxhlet extraction with acetone and 40-60 petrol then dissolved into chloroform. The solution was concentrated, then reprecipitated with acetone to yield the polymer 2 (0.428 g, 89%) as a black-green solid. GPC (chlorobenzene, 50° C.): Mn=43,000 Kg/mol, Mw=115,000 kg/mol, PDI=2.65.

### Example 5

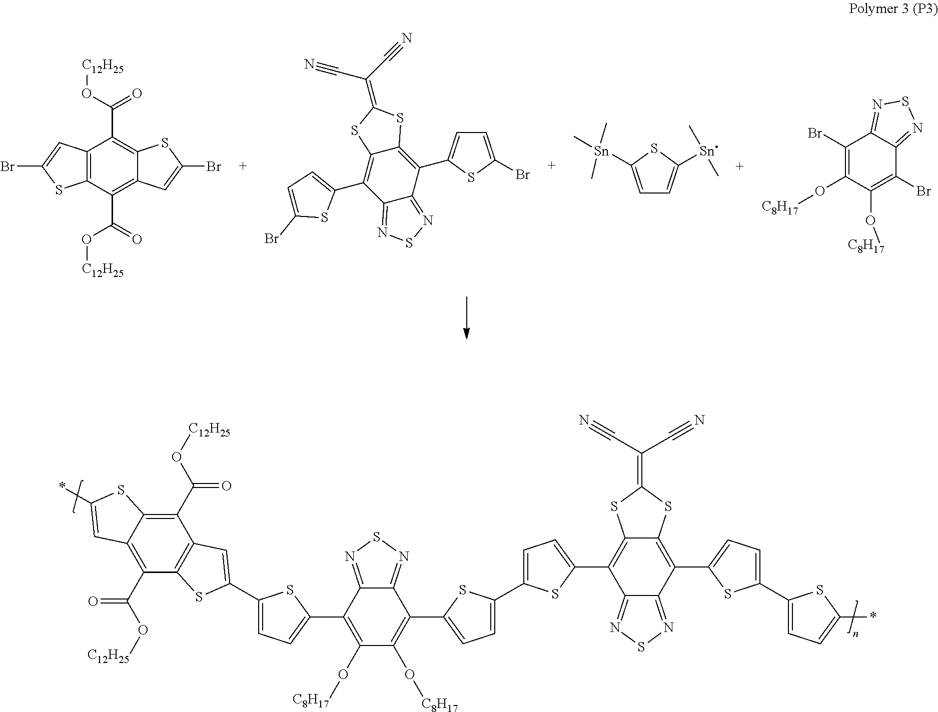

2,6-Dibromobenzo[1,2-b;4,5-b′]dithiophene-4,8-dicarboxylic acid didodecyl ester (154.55 mg; 0.20 mmol), 4,7-bis(5-bromo-2-thienyl)-(2-dicyanomethylene[1,3]dithiolo)[4,5-f]-2,1,3-benzothiadiazole (1) (119.28 mg; 0.20 mmol; 1.00 eq.), 4,7-dibromo-5,6-dioctyloxy-benzo[1,2,5]thiadiazole (110.08 mg; 0.20 mmol; 1.00 eq.), 2,5-bis(trimethylstannanyl)-thiophene (245.85 mg; 0.60 mmol; 3.00 eq.), tri-o-tolylphosphine (9.74 mg; 32.00 μmol; 0.16 eq.) and Pd₂(dba)₃ (7.33 mg; 8.00 μmol; 0.04 eq.) were weighed into a flask. Degassed chlorobenzene (2.50 cm³) was added and the mixture further purged with nitrogen for 5 minutes. The mixture was stirred at 120° C. under nitrogen for 16 hours then allowed to cool to room temperature. The reaction mixture was precipitated into methanol and the black solid was collected by suction filtration and washed with methanol. The crude polymer solid was subjected to Soxhlet extraction with acetone, petroleum ether 40-60° C., cyclohexane and chloroform. The residue was finally extracted off with chlorobenzene, precipitated in methanol again, to afford Polymer 3 as a dark-blue solid (38 mg). GPC (chlorobenzene, 50° C.): Mₙ=10,600 Kg/mol, Mw=28,200 kg/mol, PDI=2.66.

### Use Example A

### Field-Effect Transistor Fabrication and Measurements: General Procedure

Top-gate thin-film organic field-effect transistors (OFETs) were fabricated on glass substrates with vacuum evaporated Au source-drain electrodes. A 7 mg/cm³ solution of the organic semiconductor in dichlorobenzene was spin-coated on top (an optional annealing of the film is carried out at 100° C., 150° C. or 200° C. for between 1 and 5 minutes) followed by a spin-coated fluoropolymer dielectric material (Lisicon® D139 from Merck, Germany). Finally a vacuum evaporated Au gate electrode was deposited. The electrical characterization of the transistor devices was carried out in ambient air atmosphere using computer controlled Agilent 4155C Semiconductor Parameter Analyser. Charge carrier mobility in the saturation regime (μₛₐₜ) was calculated for the compound. Field-effect mobility was calculated in the saturation regime (V_{d}>(V_{g}−V₀)) using equation (1):

$${( \frac{{dI}_{d}^{sat}}{{dV}_{g}} )}_{{V}_{d}} = \frac{{WC}_{i}}{L} {μ}^{sat} ( {V}_{g} - {V}_{0} ) \qquad ( 1 )$$

where W is the channel width, L the channel length, C; the capacitance of insulating layer, V_{g} the gate voltage, V₀ the turn-on voltage, and μₛₐₜ is the charge carrier mobility in the saturation regime. Turn-on voltage (V₀) was determined as the onset of source-drain current.

1. A compound comprising one or more divalent heteroarylene units of formula I 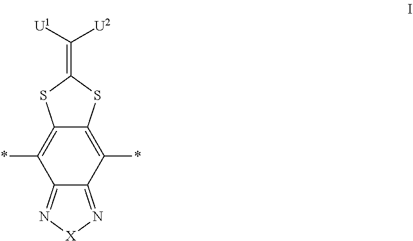
wherein the individual radicals, independently of each other and on each occurrence identically or differently, have the following meanings
X O, S, Se, Te, —NR—, —PR—, —P(═O)—, —P(OR)—, —P(O)(OR)— or —CR═CR′—
U¹, U² an electron withdrawing group, or U¹ and U² together form a carbocyclic, heterocyclic, aromatic or heteroaromatic ring having 4 to 15 ring atoms that is optionally substituted by one or more groups L, R¹ or R²,
R H or straight-chain, branched or cyclic alkyl with 1 to 25, preferably 1 to 20 C atoms, in which one or more CH₂ groups are optionally replaced by —O—, —S—, —C(═O)—, —C(═S)—, —C(═O)—O—, —O—C(═O)—, —NR⁰—, —SiR⁰R⁰⁰—, —CF₂—, —CR⁰═CR⁰⁰—, —CY¹═CY²— or —C≡C— in such a manner that O and/or S atoms are not linked directly to one another, and in which one or more H atoms are optionally replaced by F, Cl, Br, I or CN, and in which one or more CH₂ or CH₃ groups are optionally replaced by a cationic or anionic group, or aryl, heteroaryl, arylalkyl, heteroarylalkyl, aryloxy or heteroaryloxy, wherein each of the aforementioned cyclic groups has 5 to 20 ring atoms, is mono- or polycyclic, does optionally contain fused rings, and is unsubstituted or substituted by one or more identical or different groups L,
R^{1,2} H, F, Cl, CN, or straight-chain, branched or cyclic alkyl with 1 to 30 C atoms, in which one or more CH₂ groups are optionally replaced by —O—, —S—, —C(═O)—, —C(═S)—, —C(═O)—O—, —O—C(═O)—, —NR⁰—, —SiR⁰R⁰⁰—, —CF₂—, —CR⁰═CR⁰⁰—, —CY¹═CY²— or —C≡C— in such a manner that O and/or S atoms are not linked directly to one another, and in which one or more H atoms are optionally replaced by F, Cl, Br, I or CN, and in which one or more CH₂ or CH₃ groups are optionally replaced by a cationic or anionic group, or aryl, heteroaryl, arylalkyl, heteroarylalkyl, aryloxy or heteroaryloxy, wherein each of the aforementioned cyclic groups has 5 to 20 ring atoms, is mono- or polycyclic, does optionally contain fused rings, and is unsubstituted or substituted by one or more identical or different groups L,
or R¹ and R² form an aromatic or heteroaromatic ring system that is fused to the pyrazine ring to which R¹ and R² are attached, has 5 to 20 ring atoms, is mono- or polycyclic, optionally contains fused rings, and is unsubstituted or substituted by one or more identical or different groups L,
L F, Cl, —CN, —NC, —NCO, —NCS, —OCN, —SCN, R⁰, OR⁰, SR⁰, —C(═O)X⁰, —C(═O)R⁰, —C(═O)—OR⁰, —O—C(═O)—R⁰, —NH₂, —NHR⁰, —NR⁰R⁰⁰, —C(═O)NHR⁰, —C(═O)NR⁰R⁰⁰, —SO₃R⁰, —SO₂R⁰, —OH, —NO₂, —CF₃, —SF₅, or optionally substituted silyl, or carbyl or hydrocarbyl with 1 to 30, preferably 1 to 20 C atoms that is optionally substituted and optionally comprises one or more hetero atoms, preferably F, —CN, R⁰, —OR⁰, —SR⁰, —C(═O)—R⁰, —C(═O)—OR⁰, —O—C(═O)—R⁰, —O—C(═O)—OR⁰, —C(═O)—NHR⁰, —C(═O)—NR⁰R⁰⁰,
Y¹, Y² H, F, Cl or CN,
X⁰ halogen, preferably F or Cl,
R⁰, R⁰⁰ H or straight-chain or branched alkyl with 1 to 20 C atoms that is optionally fluorinated.

2. The compound of claim 1, wherein X is S, O, —NR— or —CR¹═CR²—, wherein R¹ and R² are as defined in claim 1.

3. The compound of claim 1, wherein U¹ and U² are selected from CN, C(═O)R and C(═O)OR, wherein R is as defined in claim 1.

4. The compound according to claim 1, wherein R¹ and R² are selected from the following groups or any combination thereof:
the group consisting of R, —OR and —SR wherein R is straight-chain or branched alkyl with 1 to 25 C atoms that is optionally fluorinated,
the group consisting of —C(═O)—R, —C(═O)—OR, —OC(═O)—R, —C(═O)—NHR and —C(═O)—NRRⁿ, wherein R and Rⁿ are independently of each other straight-chain or branched alkyl with 1 to 25 C atoms that is optionally fluorinated,
the group consisting of aryl, aryloxy, heteroaryl and heteroaryloxy, each of which has 5 to 20 ring atoms and optionally contains fused rings and is unsubstituted or substituted by one or more groups L as defined in claim 1,
the group consisting of F, Cl and CN.

5. The compound according to claim 1, which is a conjugated polymer comprising one or more units of formula I as defined in claim 1, and further comprising one or more arylene or heteroarylene units that have from 5 to 20 ring atoms, are mono- or polycyclic, do optionally contain fused rings, are unsubstituted or substituted by one or more identical or different groups L, R¹ or R², and are either selected of formula I or are structurally different from formula I, and wherein all the aforementioned units are directly connected to each other.

6. The compound of claim 5, which comprises one or more repeating units of formula II1 or II2, and optionally one or more repeating units of formula II3:
—(Ar¹)ₐ—U—(Ar²)_{b}—(Ar³)_{c}—(Ar⁴)_{d}— II1
—(Ar¹)ₐ—(Ar²)_{b}—U—(Ar³)_{c}—(Ar⁴)_{d}— II2
—(Ar¹)ₐ—(Ar²)_{b}—(Ar³)_{c}—(Ar⁴)_{d}— II3
wherein the individual radicals, independently of each other and on each occurrence identically or differently, have the following meanings
U a unit of formula I as defined in claim 1,
Ar¹⁻⁴ arylene or heteroarylene that has 5 to 20 ring atoms, is mono- or polycyclic, does optionally contain fused rings, is unsubstituted or substituted by one or more identical or different groups L, R¹ or R², and is different from U,
a, b, c, d 0 or 1, wherein in formula II3 a+b+c+d≥1.

7. The compound according to claim 6, which is selected of formula III: 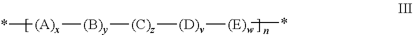
wherein the individual radicals, independently of each other and on each occurrence identically or differently, have the following meanings
A a unit of formula I, II1 or II2,
B, C, D, E a unit of formula I, II, II2 or II3,
x >0 and ≤1,
y, z, v, w ≥0 and <1,
x+y+z+v+w 1, and
n an integer ≥5.

8. The compound according to claim 7, which is selected from the following formulae

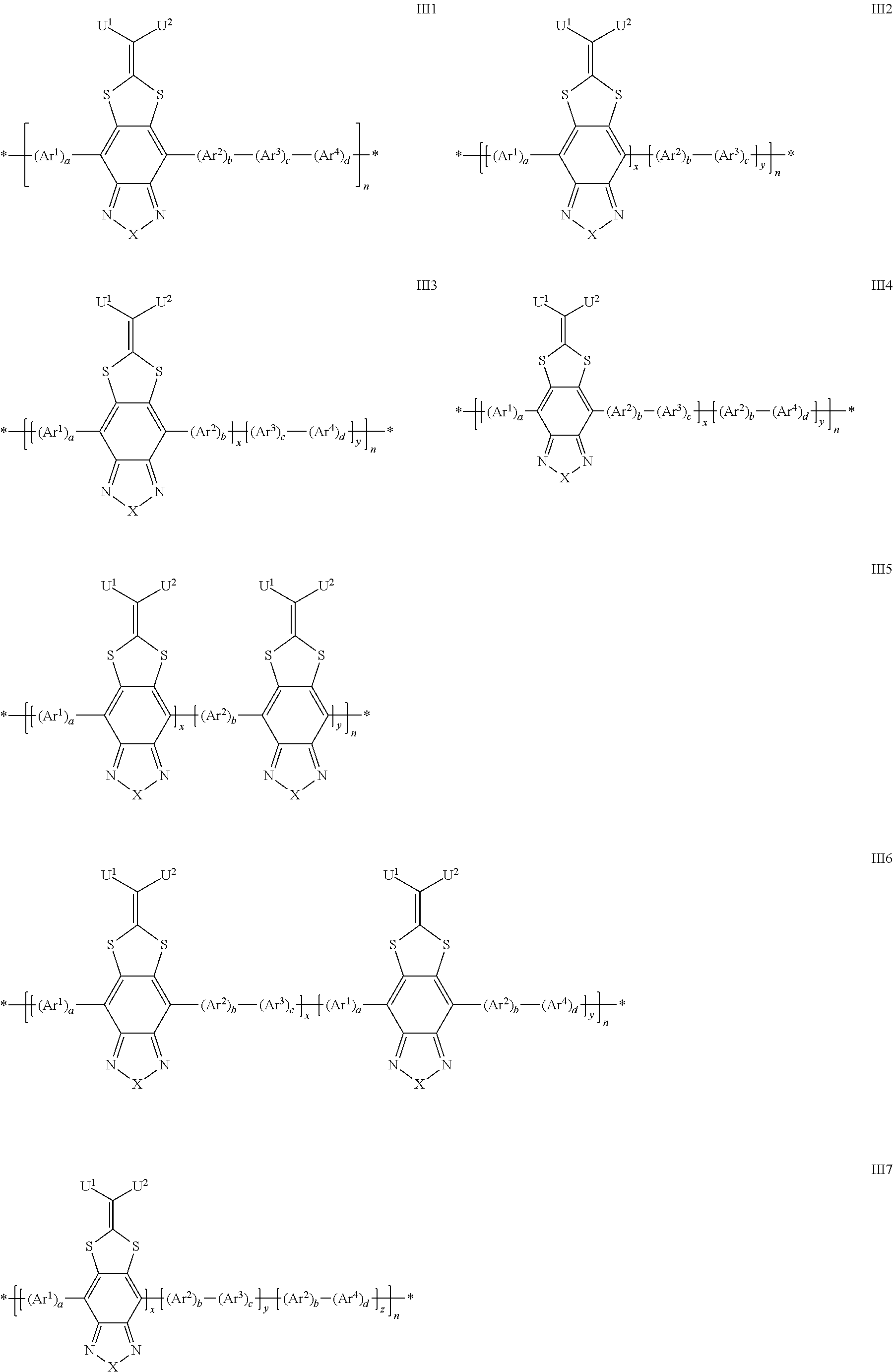

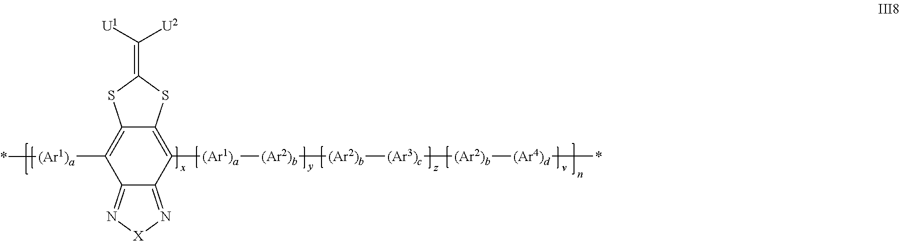

9. The compound according to claim 8, which is selected from the following formulae 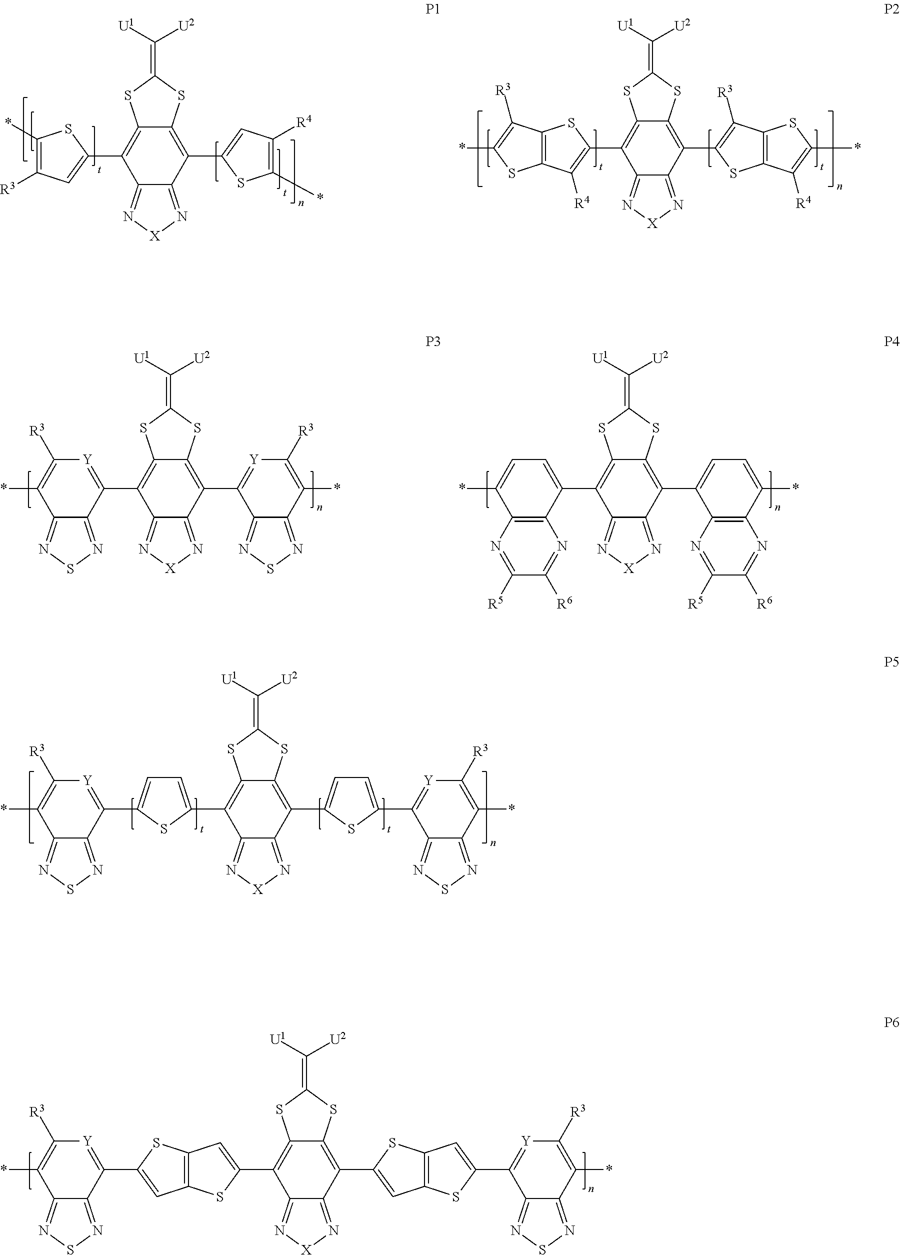 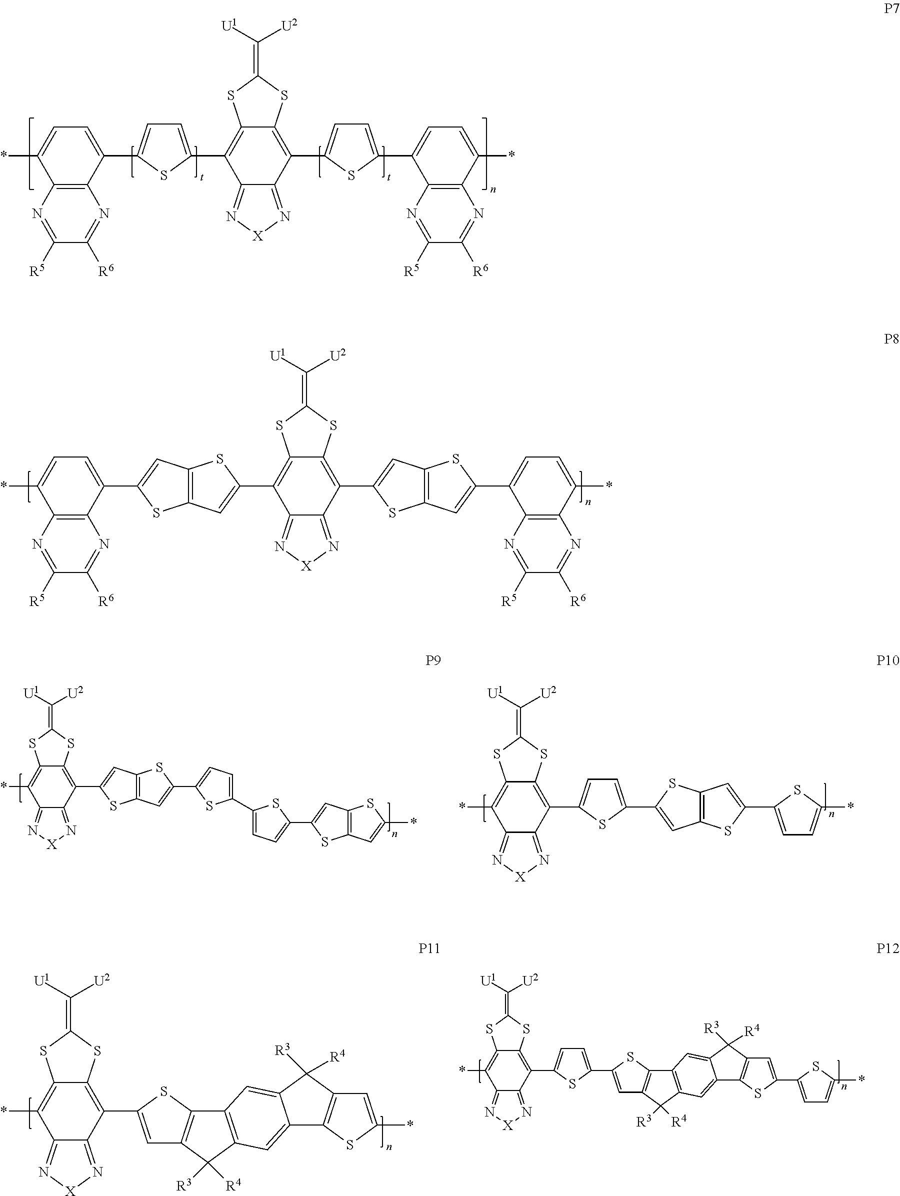 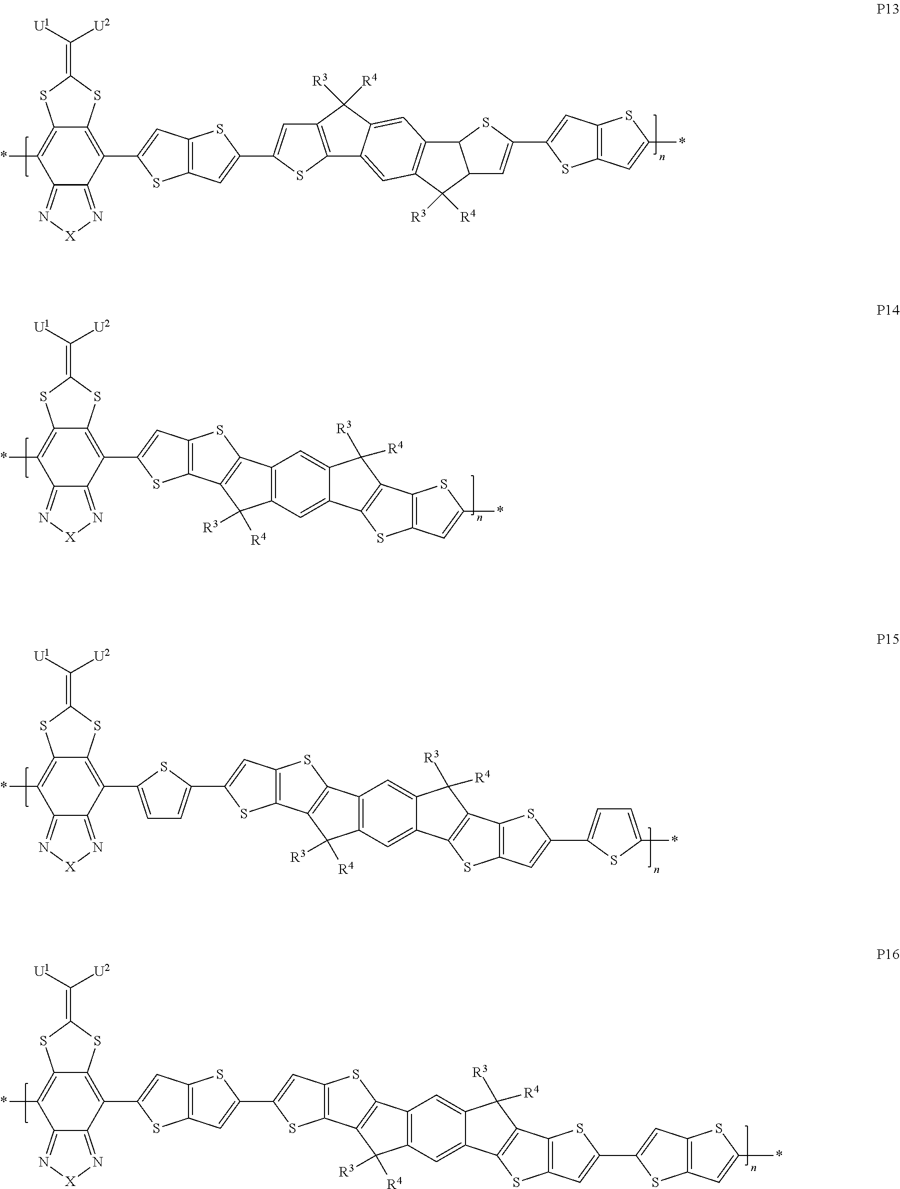 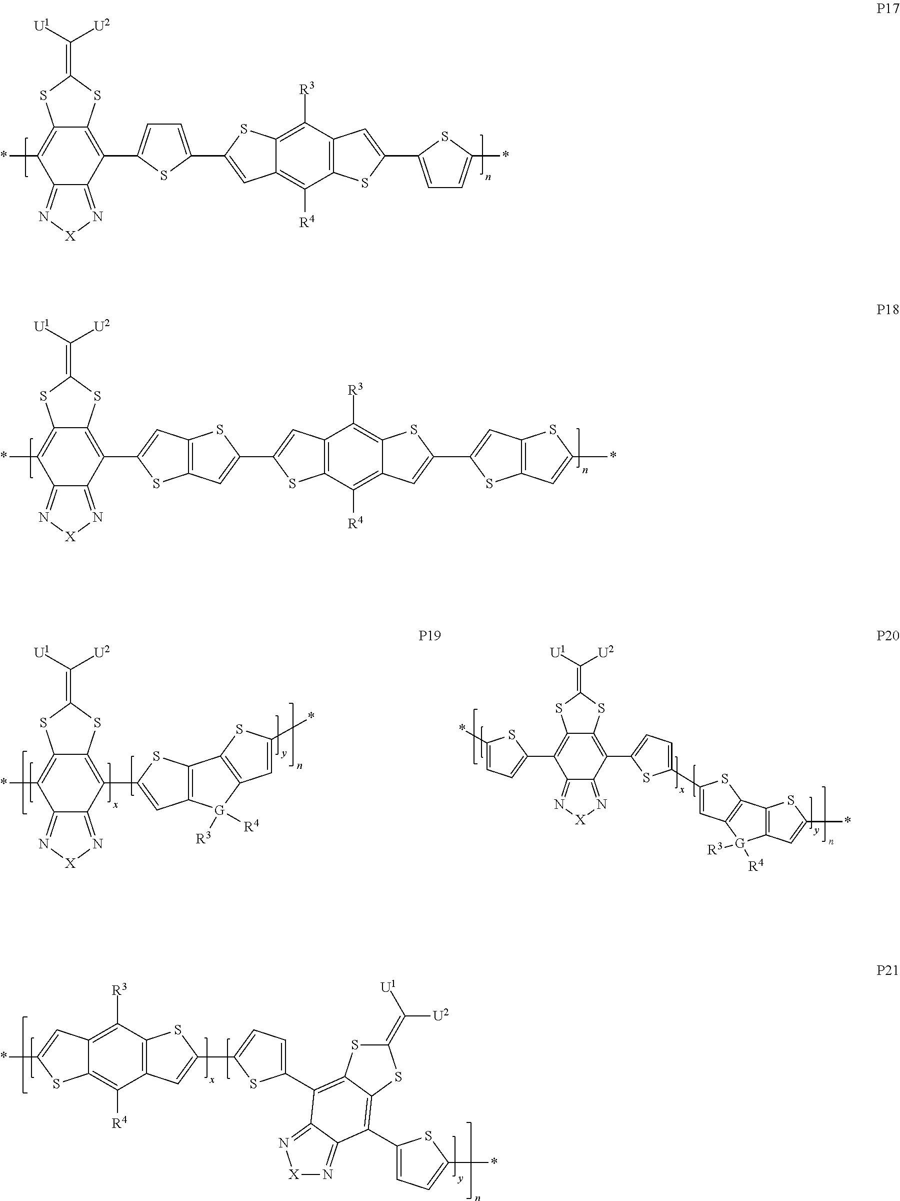 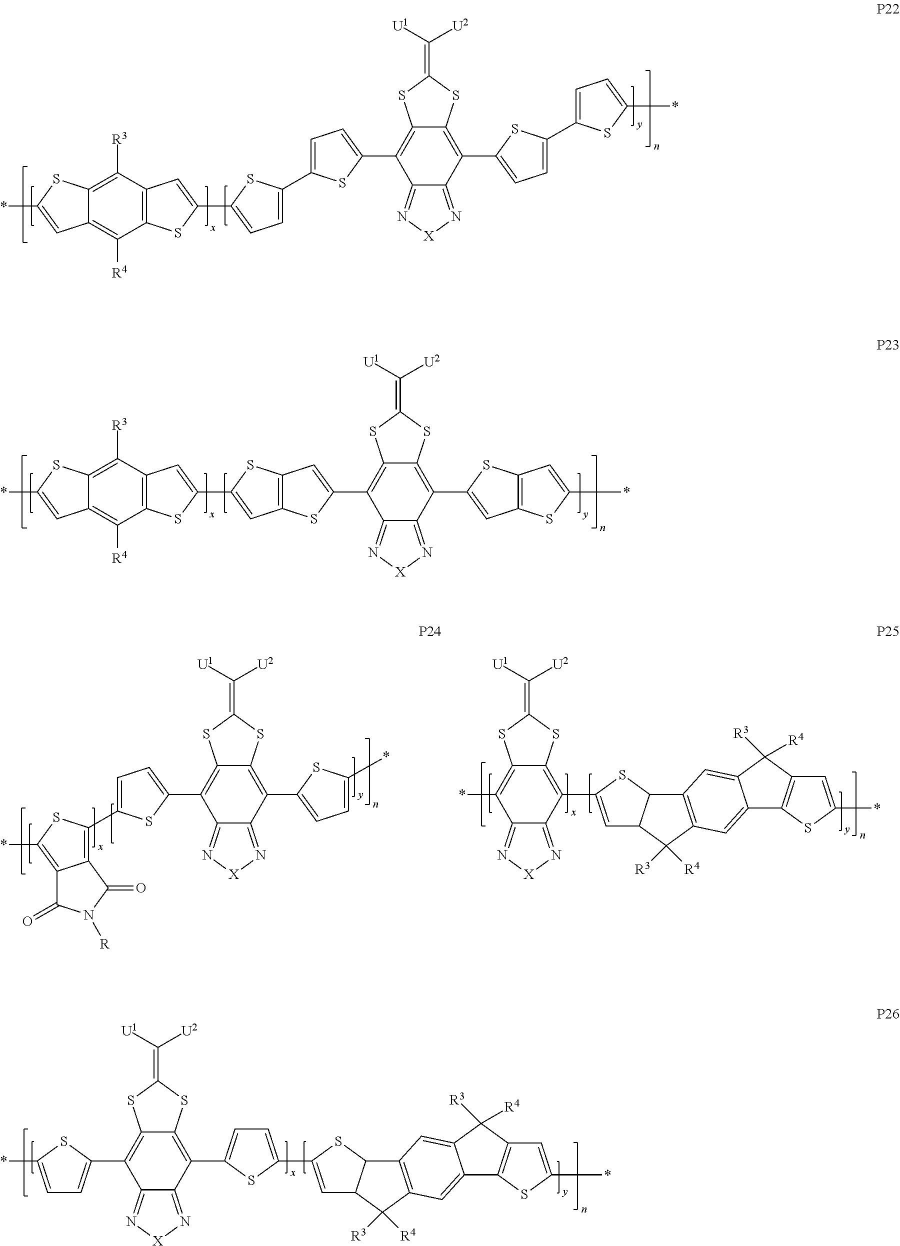 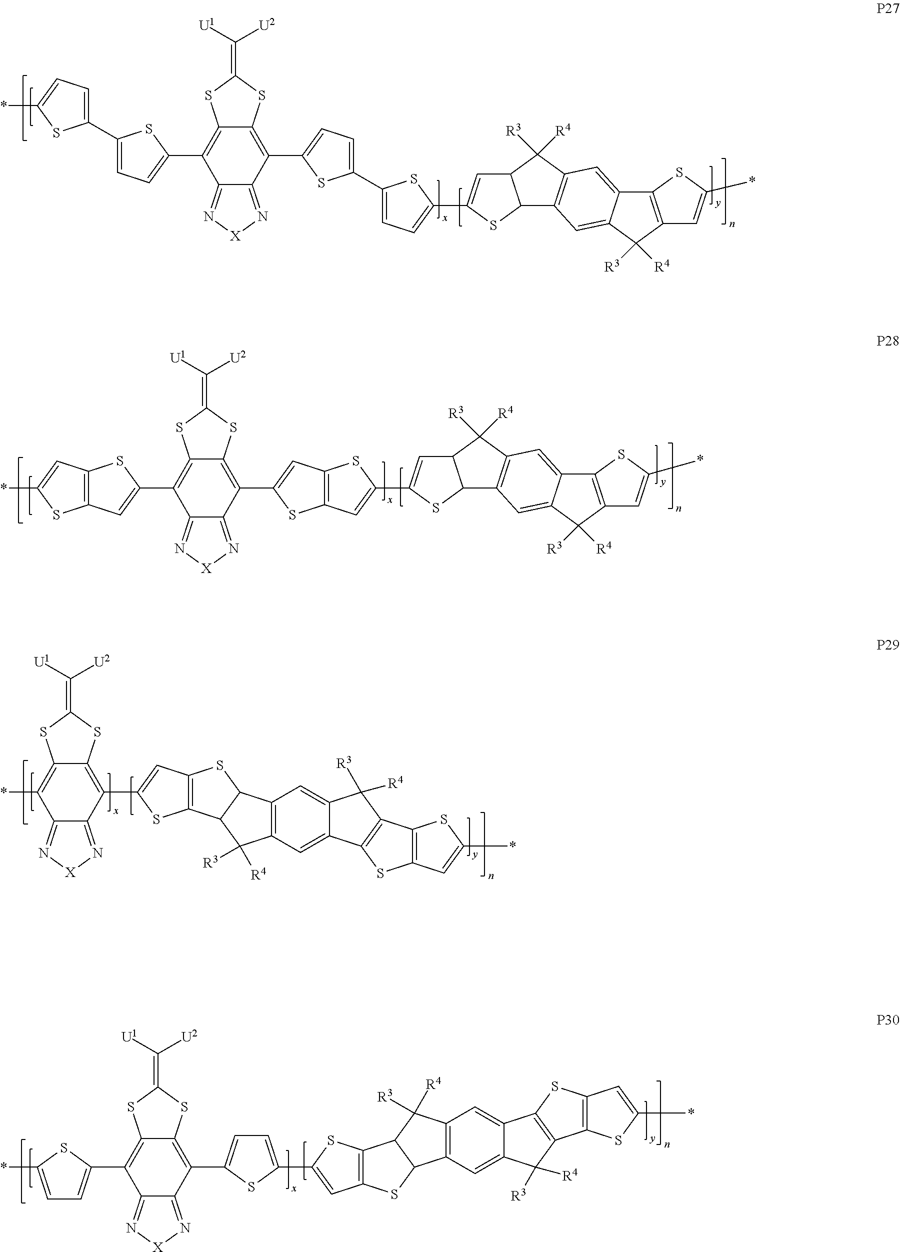 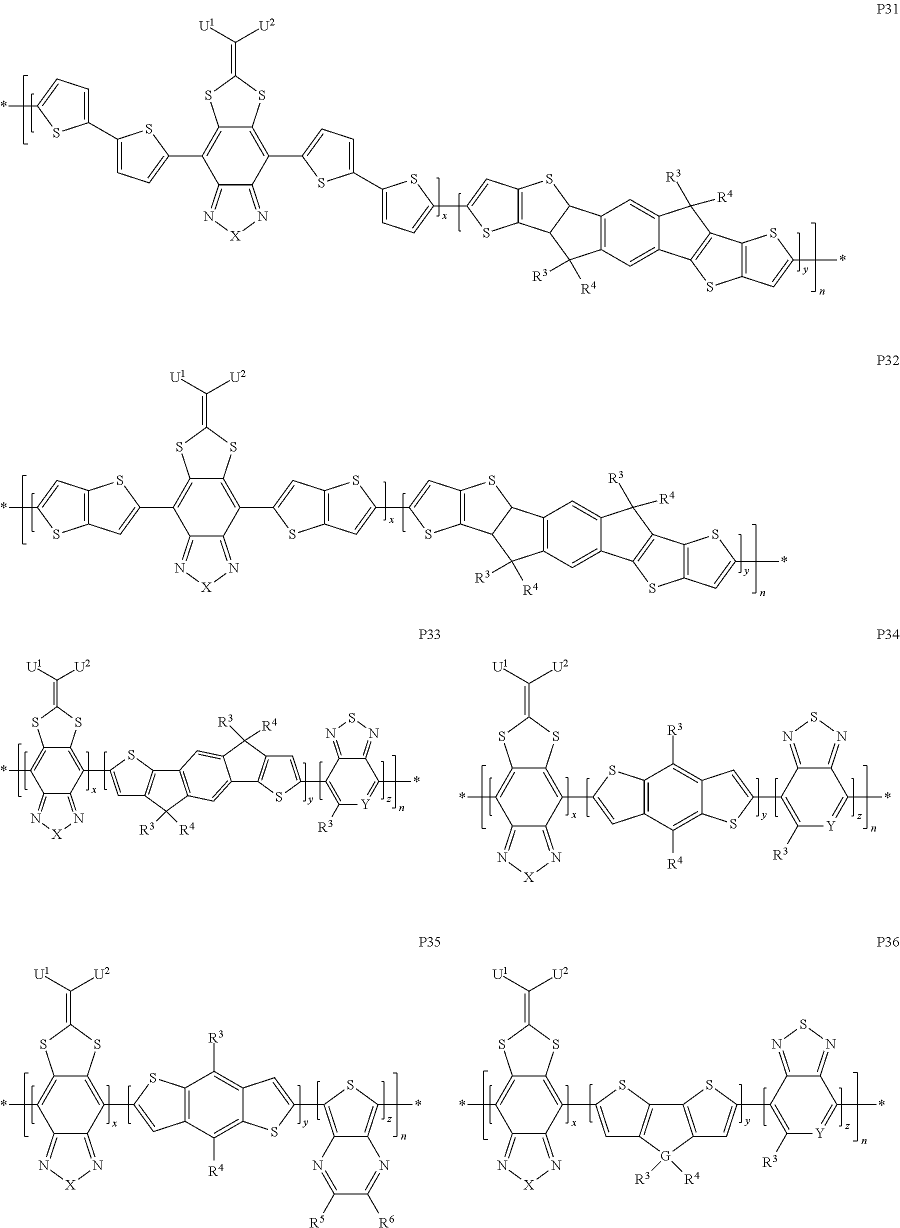 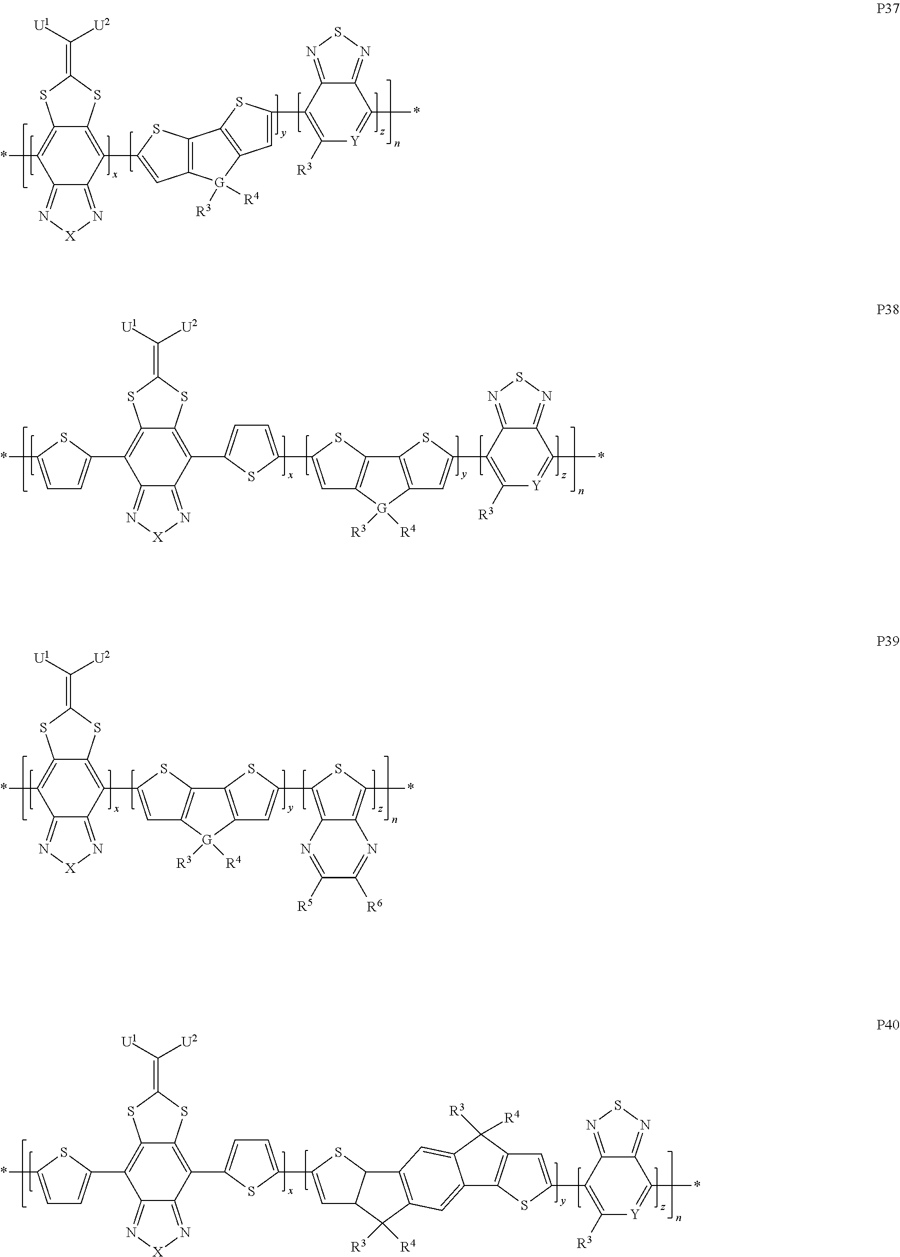 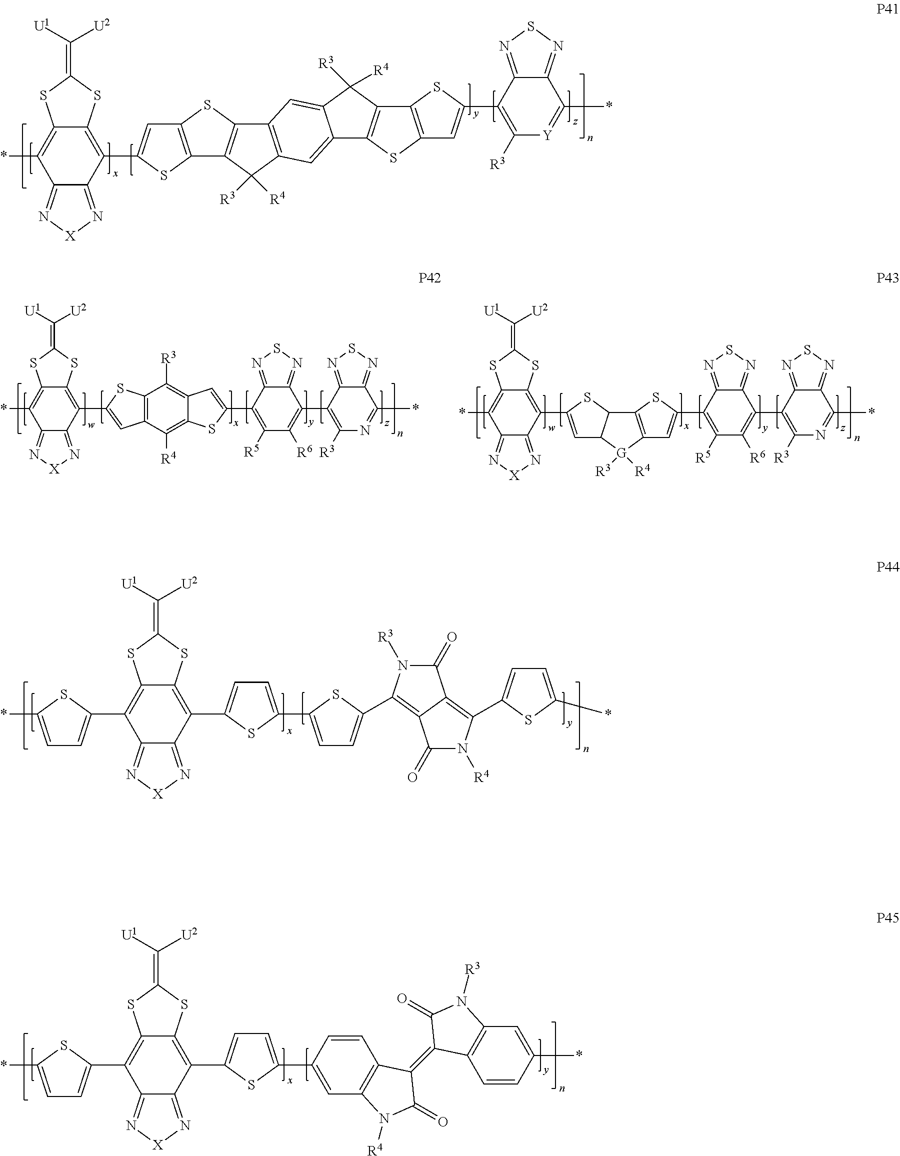
wherein Y is N or CR⁴, G is C, Si or Ge, t is 1, 2, 3 or 4, R³ and R⁴ have independently of each other and on each occurrence identically or differently one of the meanings given for R¹, and R⁵ and R⁶ have independently of each other and on each occurrence identically or differently one of the meanings given for R¹ and R².

10. The compound according to claim 7, which is selected of formula IV
R²¹-chain-R²³ IV
wherein “chain” denotes a polymer chain selected from formulae III, and R²¹ and R²² have independently of each other one of the meanings of L, or denote, independently of each other, H, F, Br, Cl, I, —CH₂Cl, —CHO, —CR′═CR″₂, —SiR′R″R′″, —SiR′X′X″, —SiR′R″X′, —SnR′R″R′″, —BR′R″, —B(OR′)(OR″), —B(OH)₂, —O—SO₂—R′, —C≡CH, —C≡C—SiR′₃, —ZnX′ or an endcap group, X′ and X″ denote halogen, R′, R″ and R′″ have independently of each other one of the meanings of R⁰, and two of R′, R″ and R′″ may also form a cyclosilyl, cyclostannyl, cycloborane or cycloboronate group with 2 to 20 C atoms together with the respective hetero atom to which they are attached.

11. A compound of formula VI
R^{T1}—(Ar¹)ₑ—(Ar²)_{f}—[(Ar³)_{g}—(Ar⁴)ₕ—U—(Ar⁵)ᵢ(Ar⁶)ₖ]ₒ—(Ar⁷)ₗ—(Ar⁸)ₘ—R^{T2} VI
wherein the individual radicals, independently of each other and on each occurrence identically or differently, have the following meanings
U a unit of formula I as defined in claim 1,
Ar¹⁻⁸ arylene or heteroarylene that has from 5 to 20 ring atoms, is mono- or polycyclic, optionally contains fused rings, and is unsubstituted or substituted by one or more identical or different groups L or R¹ as defined in claim 1, or —CY¹═CY²— or —C≡C—,
Y¹, Y² H, F, Cl or CN,
R^{T1}, R^{T2} a carbyl or hydrocarbyl group with 1 to 30 C atoms that is optionally substituted by one or more groups L and optionally comprises one or more hetero atoms,
e-m 0 or 1,
o 1, 2 or 3.

12. The compound of claim 11, wherein R^{T1} and R^{T2} are selected from H, F, Cl, Br, —NO₂, —CN, —CF₃, R*, —CF₂—R*, —O—R*, —S—R*, —SO₂—R*, —SO₃—R*, —C(═O)—H, —C(═O)—R*, —C(═S)—R*, —C(═O)—CF₂—R*, —C(═O)—OR*, —C(═S)—OR*, —O—C(═O)—R*, —O—C(═S)—R*, —C(═O)—SR*, —S—C(═O)—R*, —C(═O)NR*R**, —NR*—C(═O)—R*, —NHR*, —NR*R**, —CR*═CR*R**, —C≡C—R*, —C≡C—SiR*R**R***, —SiR*R**R***, —CH═CH(CN), —CH═C(CN)₂, —C(CN)═C(CN)₂, —CH═C(CN)(R^{a}), CH═C(CN)—C(═O)—OR*, —CH═C(CO—OR*)₂, —CH═C(CO—NR*R**)₂, and the group consisting of the following formulae 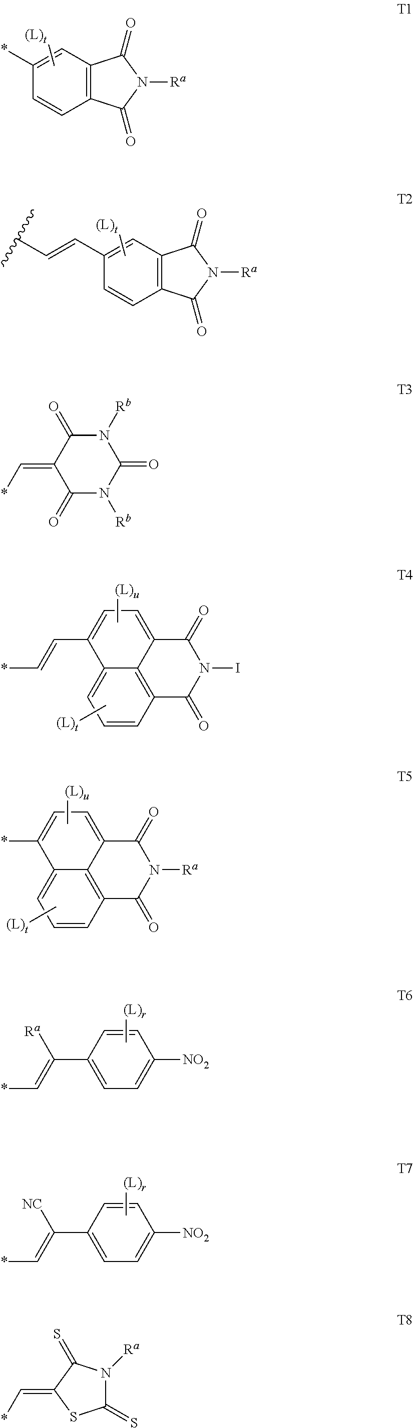 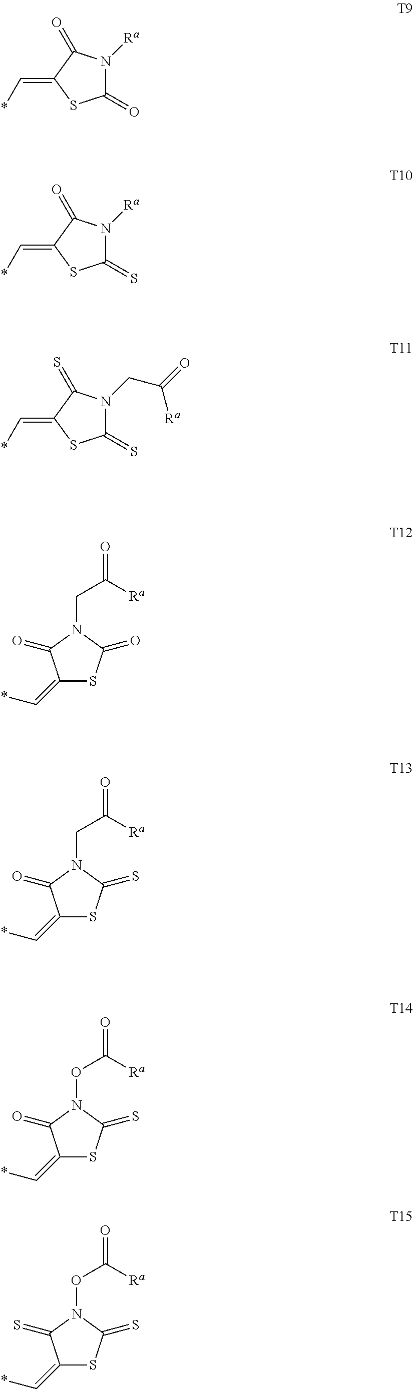 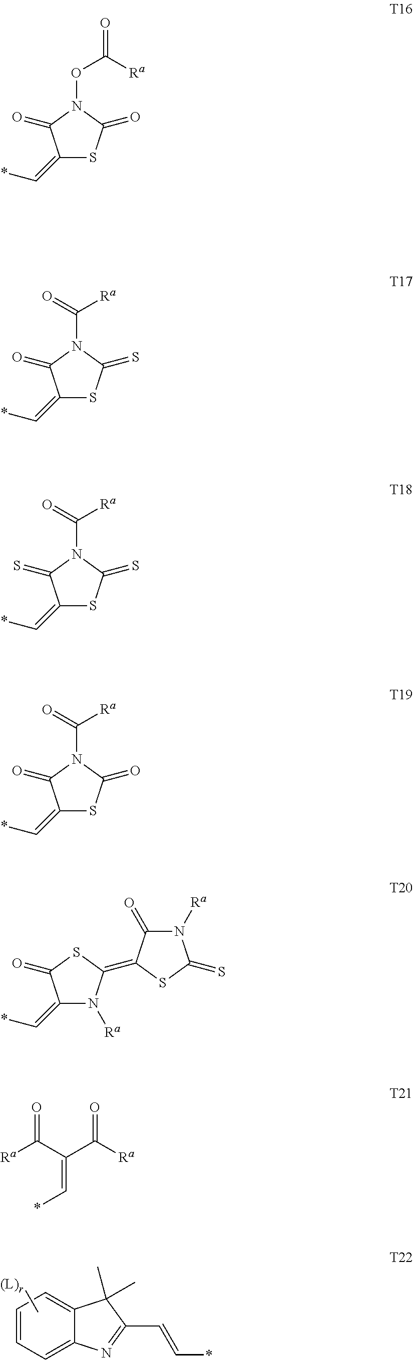 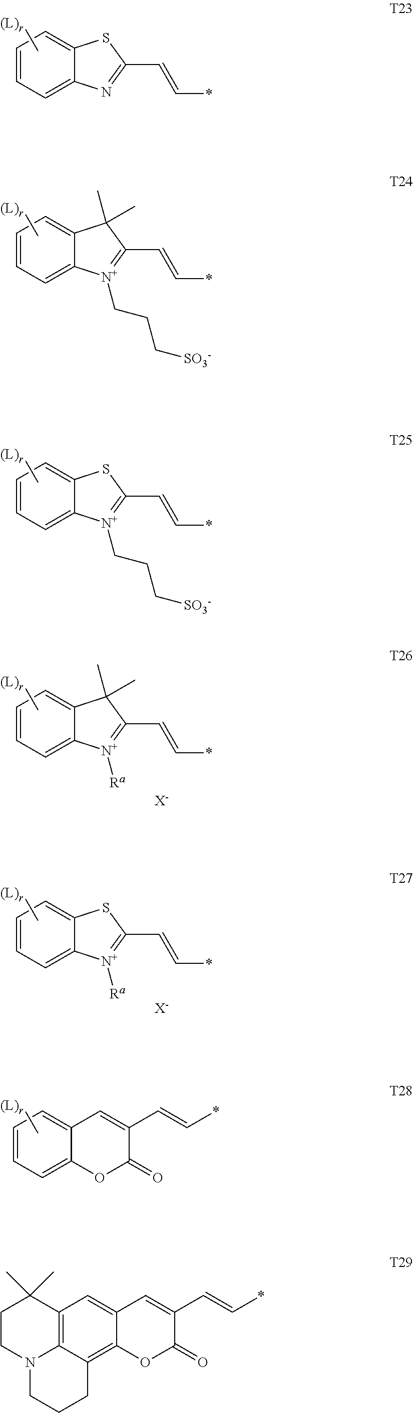 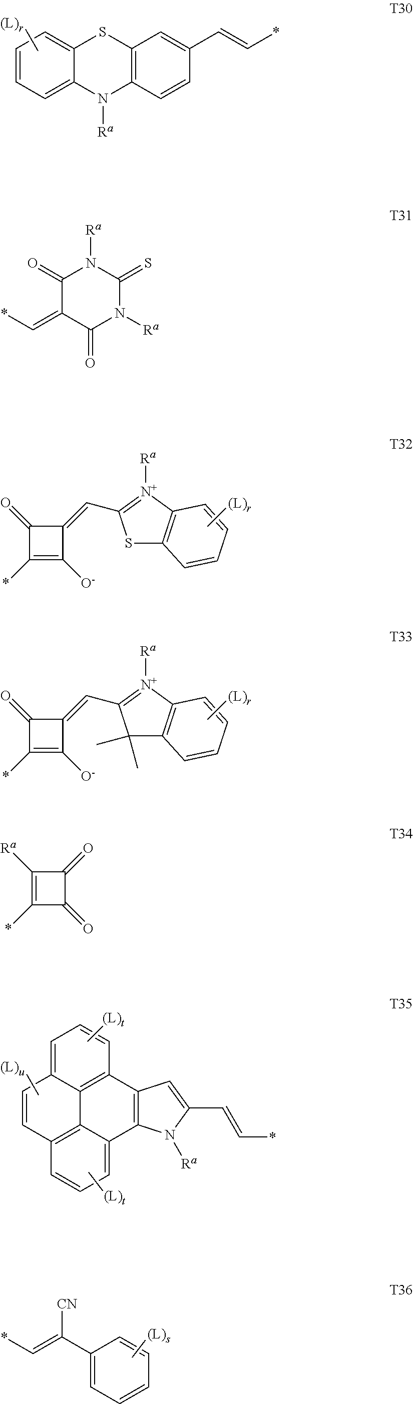 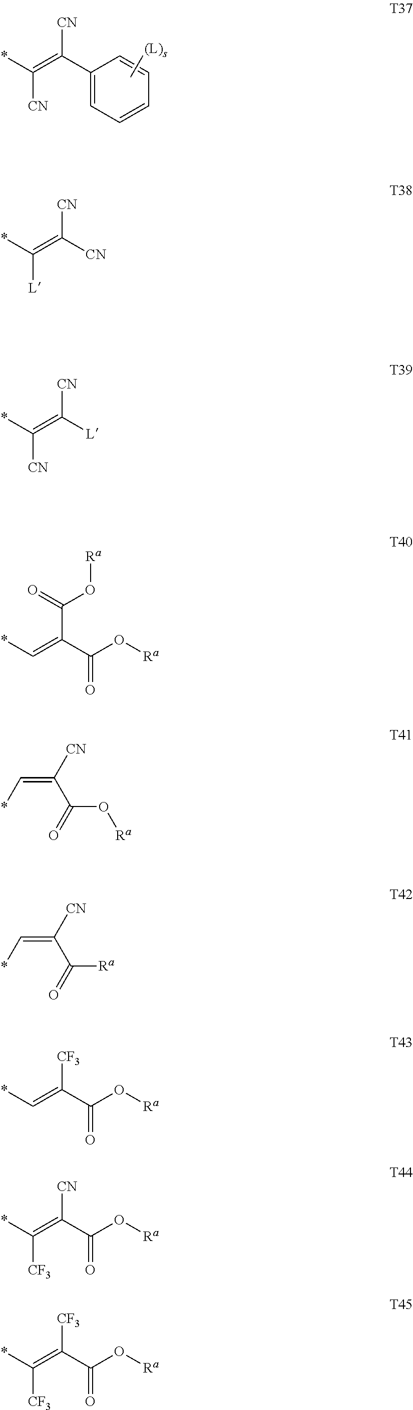 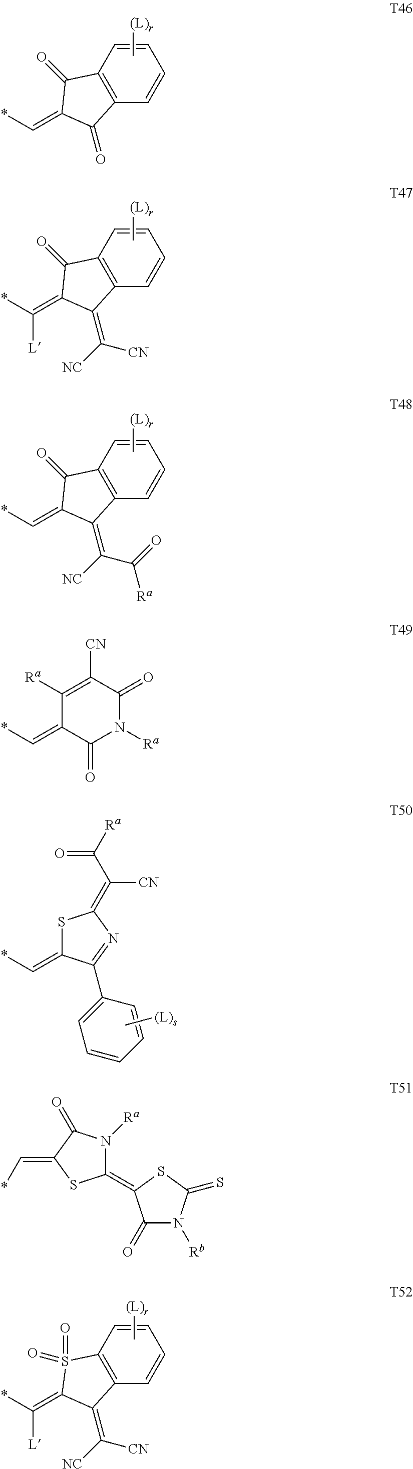
wherein the individual radicals, independently of each other and on each occurrence identically or differently, have the following meanings
R^{a}, R^{b} aryl or heteroaryl, each having from 4 to 30 ring atoms, optionally containing fused rings and being unsubstituted or substituted with one or more groups L, or one of the meanings given for L,
R*, R**, R*** alkyl with 1 to 20 C atoms which is straight-chain, branched or cyclic, and is unsubstituted, or substituted with one or more F or Cl atoms or CN groups, or perfluorinated, and in which one or more C atoms are optionally replaced by —O—, —S—, —C(═O)—, —C(═S)—, —SiR⁰R⁰⁰—, —NR⁰R⁰⁰, —CHR⁰═CR⁰⁰— or —C≡C— such that O- and/or S-atoms are not directly linked to each other,
L F, Cl, —NO₂, —CN, —NC, —NCO, —NCS, —OCN, —SCN, R⁰, OR⁰, SR⁰, —C(═O)X⁰, —C(═O)R⁰, —C(═O)—OR⁰, —O—C(═O)—R⁰, —NH₂, —NHR⁰, —NR⁰R⁰⁰, —C(═O)NHR⁰, —C(═O)NR⁰R⁰⁰, —SO₃R⁰, —SO₂R⁰, —OH, —NO₂, —CF₃, —SF₅, or optionally substituted silyl, or carbyl or hydrocarbyl with 1 to 30 C atoms that is optionally substituted and optionally comprises one or more hetero atoms,
L′ H or one of the meanings of L,
R⁰, R⁰⁰ H or straight-chain or branched alkyl with 1 to 12 C atoms that is optionally fluorinated,
Y¹, Y² H, F, Cl or CN,
X⁰ halogen,
r 0, 1, 2, 3 or 4,
s 0, 1, 2, 3, 4 or 5,
t 0, 1, 2 or 3,
u 0, 1 or 2.

13. The compound of claim 11, wherein one or both of R^{T1} and R^{T2} denote an electron withdrawing group.

14. The compound according to claim 12, wherein R^{T1} and R^{T2} are selected from the following formulae 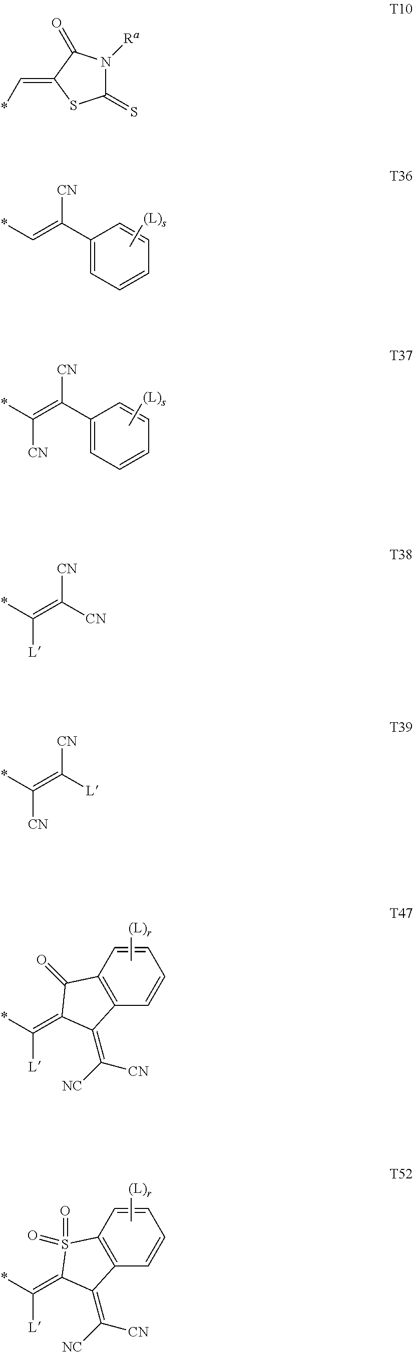
wherein L, L′, R^{a}, r and s have the meanings given in claim 12.

15. The compound according to claim 11, which is selected from the following subformulae 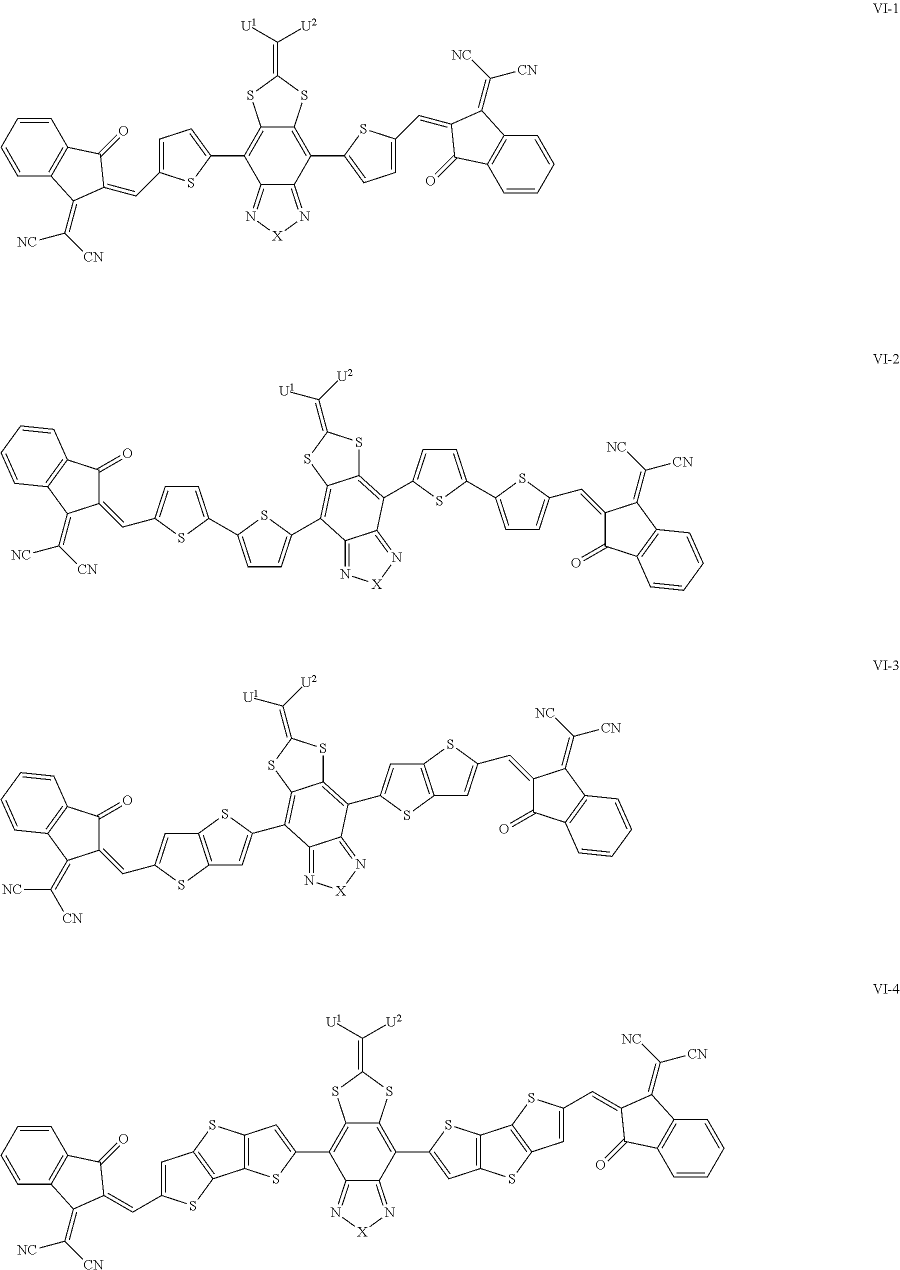 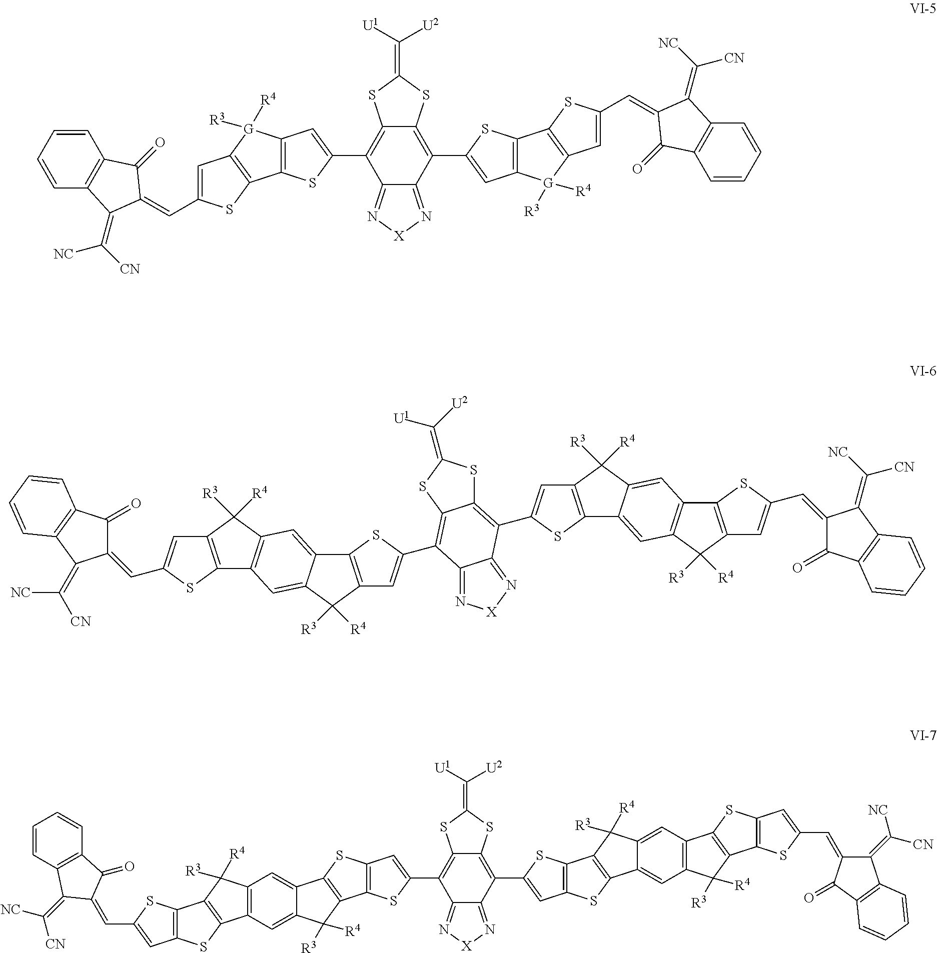
G is C, Si or Ge, t is 1, 2, 3 or 4, R³ and R⁴ have independently of each other and on each occurrence identically or differently one of the meanings given for R¹, and R⁵ and R⁶ have independently of each other and on each occurrence identically or differently one of the meanings given for R¹ and R².

16. The compound according to claim 11, which is selected from formula VI1
R^{T1}—U*—R^{T2} VI1
wherein U* is a unit selected from subformulae P1-P18

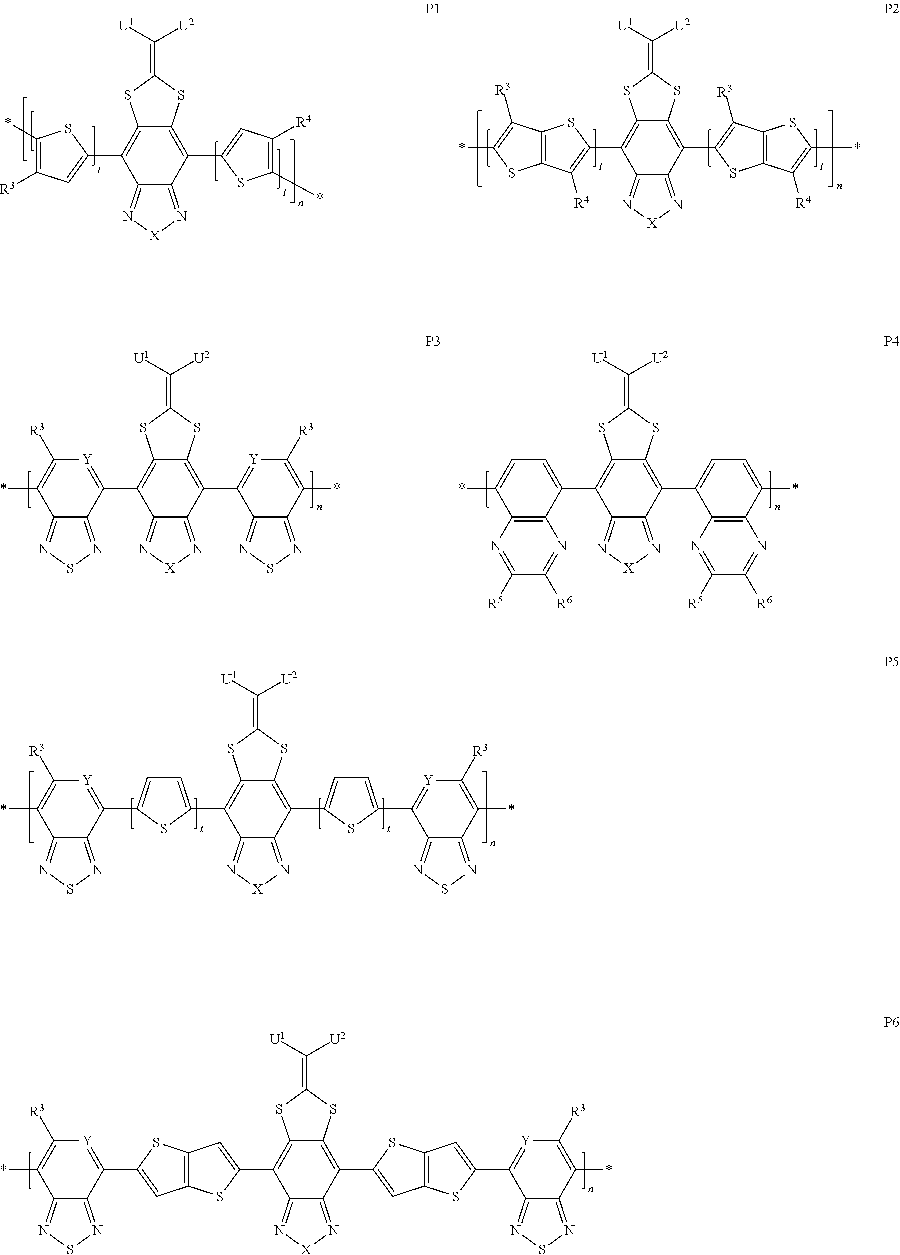

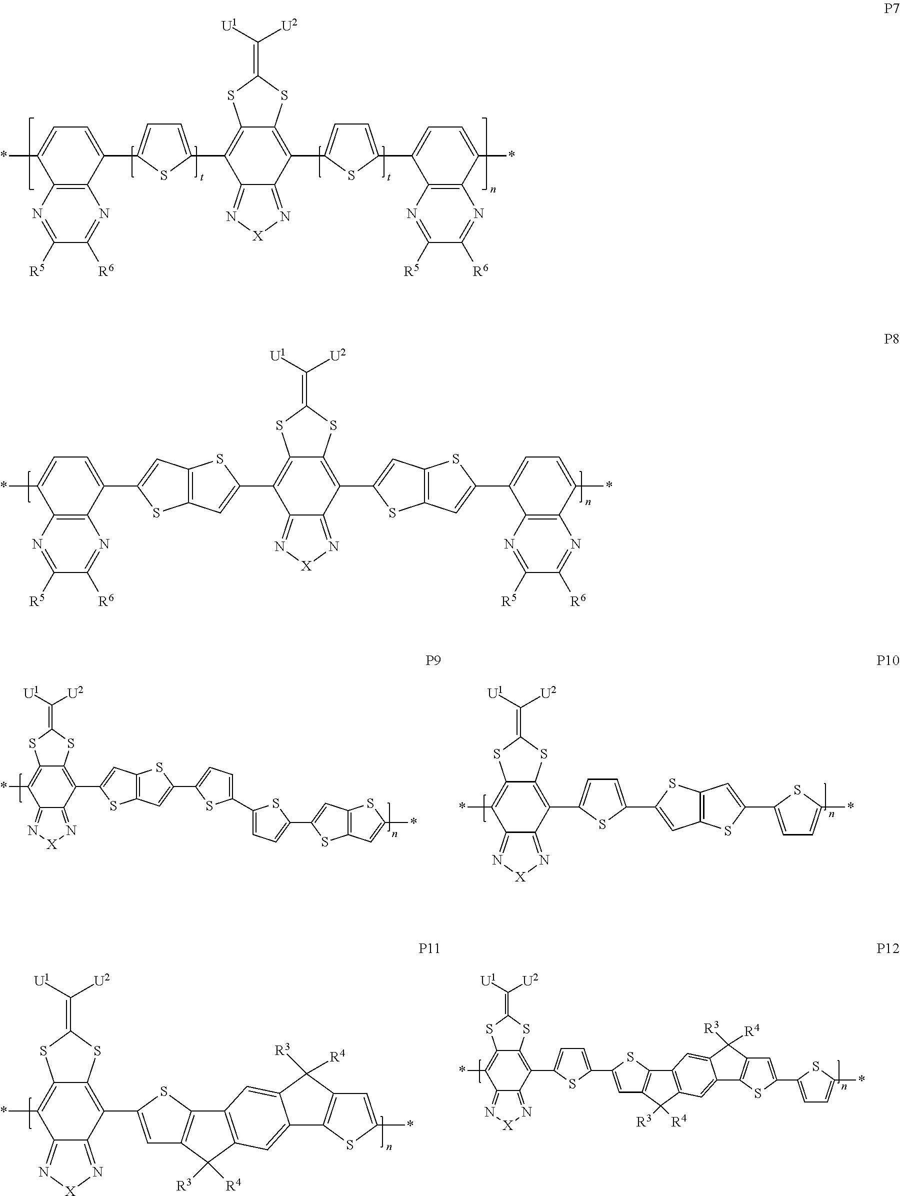

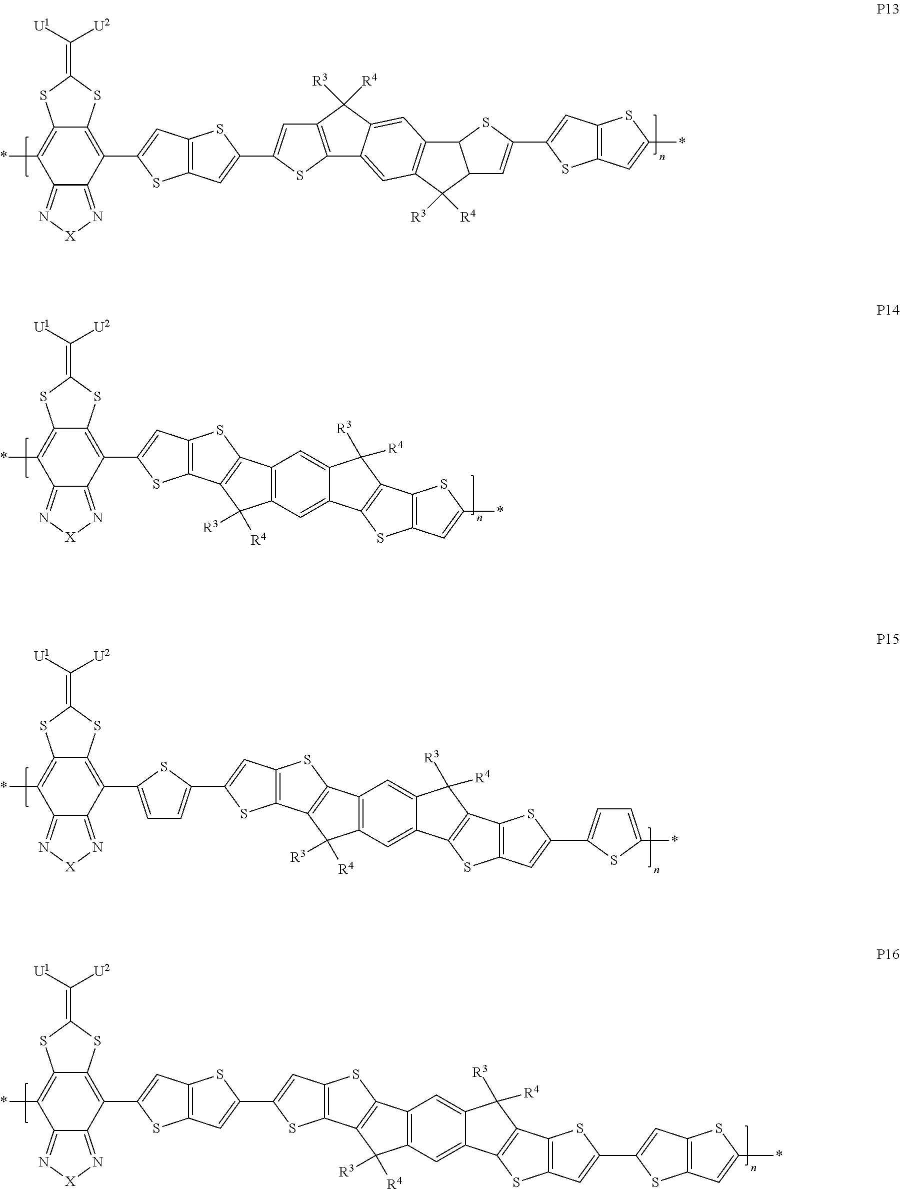

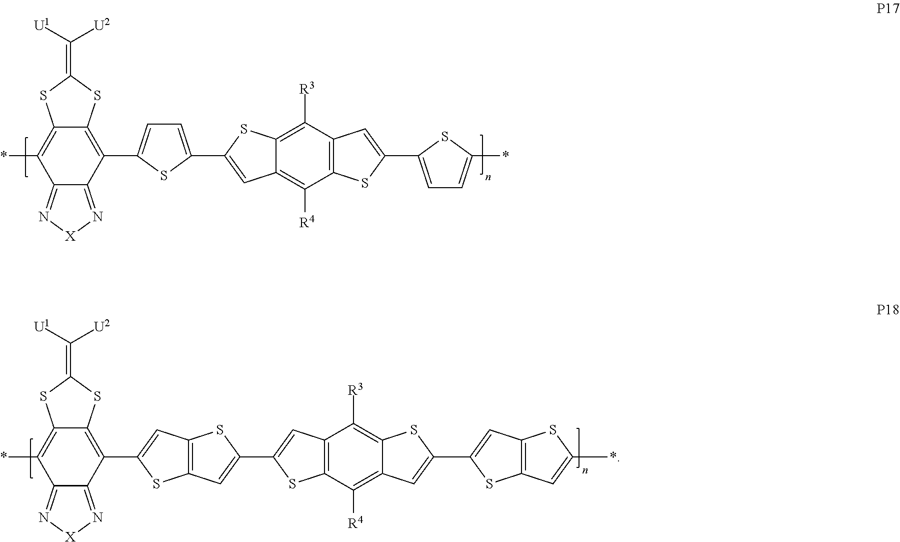

17. (canceled)

18. (canceled)

19. A composition comprising one or more compounds according to claim 1 and one or more additional compounds having one or more of semiconducting, charge transport, hole or electron transport, hole or electron blocking, electrically conducting, photoconducting or light emitting properties.

20. The composition of claim 19, wherein the one or more compounds are one or more p-type semiconductors, and further comprising one or more n-type semiconductors, preferably selected from fullerenes or fullerene derivatives.

21. The composition of claim 19, wherein the one or more compounds are one or more n-type semiconductors, and further comprising one or more p-type semiconductors, preferably selected from conjugated polymers.

22. A bulk heterojunction (BHJ) formed from a composition according to claim 19.

23. A formulation comprising one or more compounds according to claim 1, and further comprising one or more solvents selected from organic solvents.

24. An electronic or optoelectronic device, or in a component of such a device or in an assembly comprising such a device, which comprises a compound according to claim 1.

25. An electronic or optoelectronic device, or a component thereof, or an assembly comprising it, which comprises a composition according to claim 19.

26. The electronic or optoelectronic device according to claim 25, which is selected from organic field effect transistors (OFET), organic thin film transistors (OTFT), organic light emitting diodes (OLED), organic light emitting transistors (OLET), organic photovoltaic devices (OPV), organic photodetectors (OPD), organic solar cells, dye-sensitized solar cells (DSSC), perovskite-based solar cells (PSC), laser diodes, Schottky diodes, photoconductors, photodetectors and thermoelectric devices.

27. The component according to claim 25, which is selected from charge injection layers, charge transport layers, interlayers, planarising layers, antistatic films, polymer electrolyte membranes (PEM), conducting substrates and conducting patterns.

28. The assembly according to claim 25, which is selected from integrated circuits (IC), radio frequency identification (RFID) tags, security markings, security devices, flat panel displays, backlights of flat panel displays, electrophotographic devices, electrophotographic recording devices, organic memory devices, sensor devices, biosensors and biochips.

29. A process of preparing a compound according to claim 1, comprising coupling one or more compounds of formula V1 or VZ
R²³—(Ar¹)ₐ—U—(Ar²)_{b}—(Ar³)_{c}—(Ar⁴)_{d}—R²⁴ V1
R²³—(Ar¹)ₐ—(Ar²)_{b}—U—(Ar³)_{c}—(Ar⁴)_{d}—R²⁴ V2
wherein U, Ar¹⁻⁴, a, b, c and d have the meanings given in claim 6, and R²³ and R²⁴ are independently of each other selected from the group consisting of H, Cl, Br, I, O-tosylate, O-triflate, O-mesylate, O-nonaflate, —SiMe₂F, —SiMeF₂, —O—SO₂Z, —B(OZ²)₂, —CZ³═C(Z³)₂, —C≡CH, —C≡CSi(Z¹)₃, —ZnX⁰ and —Sn(Z⁴)₃, wherein X⁰ is halogen, Z¹⁻⁴ are selected from the group consisting of C₁₋₁₀ alkyl and C₆₋₁₂ aryl, each being optionally substituted, and two groups Z² may also form a cycloboronate group having 2 to 20 C atoms together with the B- and O-atoms, wherein at least one of R²³ and R²⁴ is different from H
with each other and/or with one or more monomers of formulae MI-MIV in an aryl-aryl coupling reaction
R²³—Ar¹—R²⁴ MI
R²³—Ar²—R²⁴ MII
R²³—Ar³—R²⁴ MIII
R²³—Ar⁴—R²⁴ MIV.

30. The compound according to claim 8, which is selected of formula IV
R²¹-chain-R²³ IV
wherein “chain” denotes a polymer chain selected from formulae III1-III8, and R²¹ and R²² have independently of each other one of the meanings of L, or denote, independently of each other, H, F, Br, Cl, I, —CH₂Cl, —CHO, —CR′═CR″₂, —SiR′R″R′″—SiR′X′X″, —SiR′R″X′, —SnR′R″R′″, —BR′R″, —B(OR′)(OR″), —B(OH)₂, —O—SO₂—R′, —C≡CH, —C≡C—SiR′₃, —ZnX′ or an endcap group, X′ and X″ denote halogen, R′, R″ and R′″ have independently of each other one of the meanings of R⁰, and two of R′, R″ and R′″ may also form a cyclosilyl, cyclostannyl, cycloborane or cycloboronate group with 2 to 20 C atoms together with the respective hetero atom to which they are attached.

31. The compound according to claim 9, which is selected of formula IV
R²¹-chain-R²³ IV
wherein “chain” denotes a polymer chain selected from formulae P1-P18, and R²¹ and R²² have independently of each other one of the meanings of L, or denote, independently of each other, H, F, Br, Cl, I, —CH₂Cl, —CHO, —CR′═CR″₂, —SiR′R″R′″, —SiR′X′X″, —SiR′R″X′, —SnR′R″R′″, —BR′R″, —B(OR′)(OR″), —B(OH)₂, —O—SO₂—R′, —C≡CH, —C≡C—SiR′₃, —ZnX′ or an endcap group, X′ and X″ denote halogen, R′, R″ and R′″ have independently of each other one of the meanings of R⁰, and two of R′, R″ and R′″ may also form a cyclosilyl, cyclostannyl, cycloborane or cycloboronate group with 2 to 20 C atoms together with the respective hetero atom to which they are attached.
